SEQUENCE LISTING (74 sequences)

SEQ ID NO: 1 - protein, 7 aa, Artificial Sequence
  Description of Artificial Sequence Synthetic peptide
KRRQRRR

SEQ ID NO: 2 - protein, 16 aa, Artificial Sequence
  Description of Artificial Sequence Synthetic peptide
RQIKIWFQNRRMKWKK

SEQ ID NO: 3 - protein, 34 aa, Artificial Sequence
  Description of Artificial Sequence Synthetic peptide
DAATATRGRSAASRPTERPRAPARSASRPRRPVD

SEQ ID NO: 4 - protein, 16 aa, Artificial Sequence
  Description of Artificial Sequence Synthetic peptide
AAVALLPAVLLALLAP

SEQ ID NO: 5 - protein, 16 aa, Artificial Sequence
  Description of Artificial Sequence Synthetic peptide
AAVLLPVLLPVLLAAP

SEQ ID NO: 6 - protein, 15 aa, Artificial Sequence
  Description of Artificial Sequence Synthetic peptide
VTVLALGALAGVGVG

SEQ ID NO: 7 - protein, 17 aa, Artificial Sequence
  Description of Artificial Sequence Synthetic peptide
GALFLGWLGAAGSTMGA

SEQ ID NO: 8 - protein, 17 aa, Artificial Sequence
  Description of Artificial Sequence Synthetic peptide
MGLGLHLLVLAAALQGA

SEQ ID NO: 9 - protein, 24 aa, Artificial Sequence
  Description of Artificial Sequence Synthetic peptide
LGTYTQDFNKFHTFPQTAIGVGAP

SEQ ID NO: 10 - protein, 26 aa, Artificial Sequence
  Description of Artificial Sequence Synthetic peptide
GWTLNSAGYLLKINLKALAALAKKIL

SEQ ID NO: 11 - protein, 16 aa, Artificial Sequence
  Description of Artificial Sequence Synthetic peptide
TPPKKKRKVEDPKKKK

SEQ ID NO: 12 - protein, 7 aa, Artificial Sequence
  Description of Artificial Sequence Synthetic peptide
RRRRRRR

SEQ ID NO: 13 - protein, 18 aa, Artificial Sequence
  Description of Artificial Sequence Synthetic peptide
KLALKLALKALKAALKLA

SEQ ID NO: 14 - protein, 16 aa, Artificial Sequence
  Description of Artificial Sequence Synthetic peptide
GLFGAIAGFIENGWEG

SEQ ID NO: 15 - protein, 16 aa, Artificial Sequence
  Description of Artificial Sequence Synthetic peptide
FFGAVIGTIALGVATA

SEQ ID NO: 16 - protein, 16 aa, Artificial Sequence
  Description of Artificial Sequence Synthetic peptide
FLGFLLGVGSAIASGV

SEQ ID NO: 17 - protein, 16 aa, Artificial Sequence
  Description of Artificial Sequence Synthetic peptide
GVFVLGFLGFLATAGS

SEQ ID NO: 18 - protein, 16 aa, Artificial Sequence
  Description of Artificial Sequence Synthetic peptide
GAAIGLAWIPYFGPAA

SEQ ID NO: 19 - protein, 56 aa, Artificial Sequence
  Description of Artificial Sequence Synthetic peptide
ACTCPYCKDSEGRGSGDPGKKKQHICHIQGCGKVYGKTSHLRAHLRWHTGERPFMC

SEQ ID NO: 20 - protein, 54 aa, Artificial Sequence
  Description of Artificial Sequence Synthetic peptide
ACTCPNCKDGEKRSGEQGKKKHVCHIPDCGKTFRKTSLLRAHVRLHTGERPFVC

SEQ ID NO: 21 - protein, 55 aa, Artificial Sequence
  Description of Artificial Sequence Synthetic peptide
ACTCPNCKEGGGRGTNLGKKKQHICHIPGCGKVYGKTSHLRAHLRWHSGERPFVC

SEQ ID NO: 22 - protein, 56 aa, Artificial Sequence
  Description of Artificial Sequence Synthetic peptide
ACSCPNCREGEGRGSNEPGKKKQHICHIEGCGKVYGKTSHLRAHLRWHTGERPFIC

SEQ ID NO: 23 - protein, 60 aa, Artificial Sequence
  Description of Artificial Sequence Synthetic peptide
RCTCPNCTNEMSGLPPIVGPDERGRKQHICHIPGCERLYGKASHLKTHLRWHTGERPFLC

SEQ ID NO: 24 - protein, 58 aa, Artificial Sequence
  Description of Artificial Sequence Synthetic peptide
TCDCPNCQEAERLGPAGVHLRKKNIHSCHIPGCGKVYGKTSHLKAHLRWHTGERPFVC

SEQ ID NO: 25 - protein, 53 aa, Artificial Sequence
  Description of Artificial Sequence Synthetic peptide
RCTCPNCKAIKHGDRGSQHTHLCSVPGCGKTYKKTSHLRAHLRKHTGDRPFVC

SEQ ID NO: 26 - protein, 56 aa, Artificial Sequence
  Description of Artificial Sequence Synthetic peptide
PQISLKKKIFFFIFSNFRGDGKSRIHICHLCNKTYGKTSHLRAHLRGHAGNKPFAC

SEQ ID NO: 27 - protein, 31 aa, Artificial Sequence
  Description of Artificial Sequence Synthetic peptide
WWETWKPFQCRICMRNFSTRQARRNHRRRHR

SEQ ID NO: 28 - protein, 16 aa, Artificial Sequence
  Description of Artificial Sequence Synthetic peptide
GKINLKALAALAKKIL

SEQ ID NO: 29 - protein, 16 aa, Artificial Sequence
  Description of Artificial Sequence Synthetic peptide
RVIRVWFQNKRCKDKK

SEQ ID NO: 30 - protein, 39 aa, Artificial Sequence
  Description of Artificial Sequence Synthetic peptide
GRKKRRQRRRPPQGRKKRRQRRRPPQGRKKRRQRRRPPQ

SEQ ID NO: 31 - protein, 22 aa, Artificial Sequence
  Description of Artificial Sequence Synthetic peptide
GEQIAQLIAGYIDIILKKKKSK

SEQ ID NO: 32 - protein, 18 aa, Artificial Sequence
  Description of Artificial Sequence Synthetic peptide
CNGRCGGKKKLKLLLKLL

SEQ ID NO: 33 - protein, 20 aa, Artificial Sequence
  Description of Artificial Sequence Synthetic peptide
LRKLRKRLLRLRKLRKRLLR

SEQ ID NO: 34 - protein, 18 aa, Artificial Sequence
  Description of Artificial Sequence Synthetic peptide
KLALKLALKALKAALKLA

SEQ ID NO: 35 - protein, 18 aa, Artificial Sequence
  Description of Artificial Sequence Synthetic peptide
KLALKLALKALKAALKLA

SEQ ID NO: 36 - protein, 15 aa, Artificial Sequence
  Description of Artificial Sequence Synthetic peptide
LKLLKKLLKKLLKLL

SEQ ID NO: 37 - protein, 18 aa, Artificial Sequence
  Description of Artificial Sequence Synthetic peptide
KLALKLALKALKAALKLA

SEQ ID NO: 38 - protein, 18 aa, Artificial Sequence
  Description of Artificial Sequence Synthetic peptide
ALKLAAKLAKLALKLALK

SEQ ID NO: 39 - protein, 14 aa, Artificial Sequence
  Description of Artificial Sequence Synthetic peptide
KLALKLALKALKAA

SEQ ID NO: 40 - protein, 16 aa, Artificial Sequence
  Description of Artificial Sequence Synthetic peptide
KLALKLALKALKAALK

SEQ ID NO: 41 - protein, 20 aa, Artificial Sequence
  Description of Artificial Sequence Synthetic peptide
KLALKLALKALKAALKLALK

SEQ ID NO: 42 - protein, 22 aa, Artificial Sequence
  Description of Artificial Sequence Synthetic peptide
KLALKLALKALKAALKLALKLA

SEQ ID NO: 43 - protein, 24 aa, Artificial Sequence
  Description of Artificial Sequence Synthetic peptide
KLALKLALKALKAALKLALKLALK

SEQ ID NO: 44 - protein, 26 aa, Artificial Sequence
  Description of Artificial Sequence Synthetic peptide
KLALKLALKALKAALKLALKLALKAL

SEQ ID NO: 45 - protein, 18 aa, Artificial Sequence
  Description of Artificial Sequence Synthetic peptide
RLALRLALRALRAALRLA

SEQ ID NO: 46 - protein, 18 aa, Artificial Sequence
  Description of Artificial Sequence Synthetic peptide
RLAWRLALRALRAALRLA

SEQ ID NO: 47 - protein, 18 aa, Artificial Sequence
  Description of Artificial Sequence Synthetic peptide
KLAWKLALKALKAALKLA

SEQ ID NO: 48 - protein, 18 aa, Artificial Sequence
  Description of Artificial Sequence Synthetic peptide
KLAWKLALKALKAAWKLA

SEQ ID NO: 49 - protein, 18 aa, Artificial Sequence
  Description of Artificial Sequence Synthetic peptide
KLAWKLAWKALKAAWKLA

SEQ ID NO: 50 - protein, 15 aa, Artificial Sequence
  Description of Artificial Sequence Synthetic peptide
LKLLKKLLKKLLKLL

SEQ ID NO: 51 - protein, 18 aa, Artificial Sequence
  Description of Artificial Sequence Synthetic peptide
LKTLATALTKLAKTLTTL

SEQ ID NO: 52 - protein, 18 aa, Artificial Sequence
  Description of Artificial Sequence Synthetic peptide
KLALKLALKNLKAALKLA

SEQ ID NO: 53 - protein, 18 aa, Artificial Sequence
  Description of Artificial Sequence Synthetic peptide
KALKLKAALALLAKLKLA

SEQ ID NO: 54 - protein, 18 aa, Artificial Sequence
  Description of Artificial Sequence Synthetic peptide
KLAAALLKKAKKLAAALL

SEQ ID NO: 55 - protein, 18 aa, Artificial Sequence
  Description of Artificial Sequence Synthetic peptide
KALAALLKKAAKLLAALK

SEQ ID NO: 56 - protein, 18 aa, Artificial Sequence
  Description of Artificial Sequence Synthetic peptide
KALAALLKKLAKLLAALK

SEQ ID NO: 57 - protein, 27 aa, Artificial Sequence
  Description of Artificial Sequence Synthetic peptide
WEAALAEALAEALAEHLASQPKSKRKV

SEQ ID NO: 58 - protein, 18 aa, Artificial Sequence
  Description of Artificial Sequence Synthetic peptide
KLALKLALKALKAALKLA

SEQ ID NO: 59 - protein, 18 aa, Artificial Sequence
  Description of Artificial Sequence Synthetic peptide
KLALKLALKALKAALKLA

SEQ ID NO: 60 - protein, 18 aa, Artificial Sequence
  Description of Artificial Sequence Synthetic peptide
KLALKLALKALKAALKLA

SEQ ID NO: 61 - protein, 18 aa, Artificial Sequence
  Description of Artificial Sequence Synthetic peptide
KLALKLALKALKAALKLA

SEQ ID NO: 62 - protein, 18 aa, Artificial Sequence
  Description of Artificial Sequence Synthetic peptide
KLALKLALKALKAALKLA

SEQ ID NO: 63 - protein, 27 aa, Artificial Sequence
  Description of Artificial Sequence Synthetic peptide
GWTLNSAGYLLGKINLKALAALAKKIL

SEQ ID NO: 64 - protein, 33 aa, Artificial Sequence
  Description of Artificial Sequence Synthetic peptide
LLETLLKPFQCRICMRNFSTRQARRNHRRRHRR

SEQ ID NO: 65 - protein, 28 aa, Artificial Sequence
  Description of Artificial Sequence Synthetic peptide
AAVALLPAVLLALLAPRKKRRQRRRPPQ

SEQ ID NO: 66 - protein, 29 aa, Artificial Sequence
  Description of Artificial Sequence Synthetic peptide
RKKRRQRRRPPQCAAVALLPAVLLALLAP

SEQ ID NO: 67 - protein, 16 aa, Artificial Sequence
  Description of Artificial Sequence Synthetic peptide
RQIKIWFQNRRMKWKK

SEQ ID NO: 68 - protein, 36 aa, Artificial Sequence
  Description of Artificial Sequence Synthetic peptide
KGSKKAVTKAQKKDGKKRKRSRKESYSVYVYKVLKQ

SEQ ID NO: 69 - protein, 16 aa, Artificial Sequence
  Description of Artificial Sequence Synthetic peptide
KLWSAWPSLWSSLWKP

SEQ ID NO: 70 - protein, 29 aa, Artificial Sequence
  Description of Artificial Sequence Synthetic peptide
RRRQRRKRGGDIMGEWGNEIFGAIAGFLG

SEQ ID NO: 71 - protein, 22 aa, Artificial Sequence
  Description of Artificial Sequence Synthetic peptide
GLGSLLKKAGKKLKQPKSKRKV

SEQ ID NO: 72 - protein, 29 aa, Artificial Sequence
  Description of Artificial Sequence Synthetic peptide
KETWWETWWTEWSQPGRKKRRQRRRRPPQ

SEQ ID NO: 73 - protein, 23 aa, Artificial Sequence
  Description of Artificial Sequence Synthetic peptide
CXXXXCXXXXXXXXXXXXHXXXH

SEQ ID NO: 74 - protein, 20 aa, Artificial Sequence
  Description of Artificial Sequence Synthetic peptide
LRKLRKRLLRLRKLRKRLLR

(19) United States
(12) Patent Application Publication
(10) Pub. No.: US 2009/0220435 A1
(43) Pub. Date: Sep. 3, 2009

(54) TIGHT JUNCTION MODULATING PEPTIDE COMPONENTS FOR ENHANCING MUCOSAL DELIVERY OF THERAPEUTIC AGENTS

(75) Inventors: Steven C. Quay, Seattle, WA (US); Shu-Chih Chen Quay, Seattle, WA (US); Kunyuan Cui, Bothell, WA (US); Anthony P. Sileno, Brookhaven Hamlet, NY (US); Paul Hickok Johnson, Snohomish, WA (US); Michael E. Houston, Sammamish, WA (US); Henry R. Costantino, Woodinville, WA (US); Michael V. Templin, Bothell, WA (US)

Correspondence Address: NASTECH PHARMACEUTICAL COMPANY INC;MDRNA, Inc., 3830 MONTE VILLA PARKWAY, BOTHELL, WA, 98021-7266, US

(73) Assignee: NASTECH PHARMACEUTICAL COMPANY INC., Bothell, WA (US)

(21) Appl. No.: 11/997,132

(22) PCT Filed: Jul. 27, 2006
(86) PCT No.: PCT/US06/29768

§ 371 Date: Jan. 28, 2008

Related U.S. Application Data
(60) Provisional application No. 60/703,291, filed on Jul. 27, 2005.
(60) Provisional application No. 60/703,289, filed on Jul. 27, 2005.
(60) Provisional application No. 60/710,637, filed on Aug. 22, 2005.
(60) Provisional application No. 60/750,886, filed on Dec. 16, 2005.
(60) Provisional application No. 60/772,435, filed on Feb. 10, 2006.

(51) Int. Cl. A61K 9/12; A61K 38/21; A61K 38/20; A61K 39/395; A61K 39/00; A61K 38/28; A61K 38/10; A61K 38/16; A61K 31/7088; A61K 31/60; A61K 31/56; A61K 31/485; C07K 14/00; C07K 7/00

(52) U.S. Cl. 424/45; 424/85.2; 424/85.6; 424/85.7; 424/130.1; 424/133.1; 424/141.1; 424/185.1; 514/3; 514/12; 514/13; 514/14; 514/44 R; 514/165; 514/171; 514/282; 530/325; 530/326

(57) ABSTRACT

Compounds and components including sequences for mucosal epithelial transport of an active agent are given. Tight junction modulating peptide components are described for use in transport and delivery. Permeability can be enhanced with reversibility. Compounds and components for enhanced delivery may be peptide or protein variants, conjugates, or other analog types and structures.

### BACKGROUND OF THE INVENTION

A fundamental concern in the treatment of any disease or condition is ensuring the safe and effective delivery of a therapeutic agent drug to the subject. Traditional routes of delivery for therapeutic agents include intravenous injection and oral administration. However, these delivery methods suffer from disadvantages and therefore alternative delivery systems are needed.

A major disadvantage of drug administration by injection is that trained personnel are often required to administer the drug. Additionally, trained personal are at risk when administering a drug by injection. For self-administered drugs, many patients are reluctant or unable to give themselves injections on a regular basis. Injection is also associated with increased risks of infection. Other disadvantages of drug injection include variability of delivery results between individuals, as well as unpredictable intensity and duration of drug action.

Despite disadvantages, injection remains the only approved delivery mode for many important therapeutic compounds. These include conventional drugs, as well as a rapidly expanding list of peptide and protein biotherapeutics. Delivery of these compounds via alternate routes of administration, for example, oral, nasal and other mucosal routes, is desirable, but may provide less bioavailability. For macromolecular species, for example, peptide and protein therapeutic compounds, alternate routes of administration may be limited by susceptibility to inactivation and poor absorption across mucosal barriers.

The oral administration of some therapeutic agents exhibits very low bioavailability and considerable time delay in action due to hepatic first-pass metabolism and/or degradation in the gastrointestinal tract.

Mucosal administration of therapeutic compounds offers certain advantages over injection and other modes of administration, for example, in terms of convenience and speed of delivery, as well as by reducing or eliminating compliance problems and side effects. However, mucosal delivery of biologically active agents is limited by mucosal barrier functions and other factors.

Epithelial cells make up the mucosal barrier and provide a crucial interface between the external environment and mucosal and submucosal tissues and extracellular compartments. One of the most important functions of mucosal epithelial cells is to determine and regulate mucosal permeability. In this context, epithelial cells create selective permeability barriers between different physiological compartments. Selective permeability is the result of regulated transport of molecules through the cytoplasm (the transcellular pathway) and the regulated permeability of the spaces between the cells (the paracellular pathway).

Intercellular junctions between epithelial cells are known to be involved in both the maintenance and regulation of the epithelial barrier function, and cell-cell adhesion. Tight junctions (TJ) of epithelial and endothelial cells are particularly important for cell-cell junctions that regulate permeability of the paracellular pathway, and also divide the cell surface into apical and basolateral compartments. Tight junctions form continuous circumferential intercellular contacts between epithelial cells and create a regulated barrier to the paracellular movement of water, solutes, and immune cells. They also provide a second type of barrier that contributes to cell polarity by limiting exchange of membrane lipids between the apical and basolateral membrane domains.

Tight junctions are thought to be directly involved in barrier and fence functions of epithelial cells by creating an intercellular seal to generate a primary barrier against the diffusion of solutes through the paracellular pathway, and by acting as a boundary between the apical and basolateral plasma membrane domains to create and maintain cell polarity, respectively. Tight junctions are also implicated in the transmigration of leukocytes to reach inflammatory sites. In response to chemo-attractants, leukocytes emigrate from the blood by crossing the endothelium and, in the case of mucosal infections, cross the inflamed epithelium. Transmigration occurs primarily along the paracellular rout and appears to be regulated via opening and closing of tight junctions in a highly coordinated and reversible manner.

Numerous proteins have been identified in association with TJs, including both integral and peripheral plasma membrane proteins. Current understanding of the complex structure and interactive functions of these proteins remains limited. Among the many proteins associated with epithelial junctions, several categories of trans-epithelial membrane proteins have been identified that may function in the physiological regulation of epithelial junctions. These include a number of “junctional adhesion molecules” (JAMs) and other TJ-associated molecules designated as occludins, claudins, and zonulin.

JAMs, occludin, and claudin extend into the paracellular space, and these proteins in particular have been contemplated as candidates for creating an epithelial barrier between adjacent epithelial cells and channels through epithelial cell layers. In one model, occludin, claudin, and JAM have been proposed to interact as homophilic binding partners to create a regulated barrier to paracellular movement of water, solutes, and immune cells between epithelial cells.

In the context of drug delivery, the ability of drugs to permeate epithelial cell layers of mucosal surfaces, unassisted by delivery-enhancing agents, appears to be related to a number of factors; including molecular size, lipid solubility, and ionization. In general, small molecules, less than about 300-1,000 daltons, are often capable of penetrating mucosal barriers, however, as molecular size increases, permeability decreases rapidly. For these reasons, mucosal drug administration typically requires larger amounts of drug than administration by injection. Other therapeutic compounds, including large molecule drugs, are often refractory to mucosal delivery. In addition to poor intrinsic permeability, large macromolecular drugs are often subject to limited diffusion, as well as lumenal and cellular enzymatic degradation and rapid clearance at mucosal sites. Thus, in order to deliver these larger molecules in therapeutically effective amounts, cell permeation enhancing agents are required to aid their passage across these mucosal surfaces and into systemic circulation where they may quickly act on the target tissue.

Mucosal tissues provide a substantial barrier to the free diffusion of macromolecules, while enzymatic activities present in mucosal secretions can severely limit the bioavailability of therapeutic agents, particularly peptides and proteins. At certain mucosal sites, such as the nasal mucosa, the typical residence time of proteins and other macromolecular species delivered is limited, e.g., to about 15-30 minutes or less, due to rapid mucociliary clearance.

There has been a long-standing and unmet need in the art for pharmaceutical formulations and methods of administering therapeutic compounds which provide enhanced mucosal delivery, including targeted tissues and physiological compartments such as in the central nervous system.

More specifically, there is a need in the art for safe and reliable methods and compositions for mucosal delivery of therapeutic compounds for treatment of diseases and other adverse conditions in mammalian subjects. A related need exists for methods and compositions that will provide efficient delivery of macromolecular drugs via one or more mucosal routes in therapeutic amounts, which are fast acting, easily administered and have limited adverse side effects such as mucosal irritation or tissue damage.

A need also persists in the art for methods and compositions to enhance mucosal delivery of biotherapeutic compounds that will overcome mucosal epithelial barrier mechanisms. Selective permeability of mucosal epithelia has heretofore presented major obstacles to mucosal delivery of therapeutic macromolecules, including biologically active peptides and proteins. Accordingly, there remains an unmet need in the art for new methods and tools to facilitate mucosal delivery of biotherapeutic compounds. In particular, there is a need in the art for new methods and formulations to facilitate mucosal delivery of biotherapeutic compounds that have heretofore proven refractory to delivery across mucosal barriers.

### BRIEF DESCRIPTION OF THE DRAWINGS

FIG. 1 illustrates the effects of PN159 on permeation of PTH₁₋₃₄, using PN159 with additional enhancers (Me-β-CD, DDPC, EDTA).

FIG. 2 illustrates the effects of PN159 on permeation of PTH₁₋₃₄, using PN159 without additional enhancers.

FIG. 3 illustrates the effects of PN159 on in vivo permeation of peptide YY.

FIG. 4 illustrates the effects of PN159 on permeation of an MC-4 receptor agonist.

FIG. 5 shows the effects of 25-100 μM PN159 on 40 mg/ml Galantamine lactate in vitro permeation of an epithelial monolayer.

FIG. 6 shows the chemical stability of TJM peptide at (A) 5° C., (B) 25° C., and (C) 40° C. Data are presented for pH 4.0, pH 7.3 and pH 9.0 as filled diamonds, open squares, and filled triangles, respectively.

FIG. 7 illustrates permeation kinetics of FITC-dextran MW4000 in the presence of each tight junction modulating peptide (TJMP). The PYY formulation acted as a positive control and phosphate buffered saline (PBS) was a negative control. Cell permeation was assayed after a 15-minute treatment of the cells and also after a 60-minute treatment of the cells with the TJMP and the FITC-dextran MW4000. The graph shows that permeation is dependent on the length of time the TJMP is in contact with the epithelial cell and that all TJMPs tested enhance the permeation of the FITC-dextran MW4000.

FIG. 8 illustrates transepithelial electric resistance (TER) decreases following 1-hour treatment of PN159 and PEG-PN159.

FIG. 9 illustrates permeability of FITC dextran 3000 increases following treatment with PN159 and PEG-PN159.

FIG. 10 illustrates the permeation ratio of PN159 and PEG-PN159.

FIG. 11 illustrates pegylation of PN159 reduces toxicity (LDH assay).

FIG. 12 illustrates enhanced mean plasma PYY₃₋₃₆ concentration following nasal administration with PEGylated peptide PN529 (PEG-PN159).

FIG. 13 illustrates enhanced mean plasma PYY3-36 concentration following nasal administration with PEGylated peptide PN529 (PEG-PN159) (Log-Linear Plot).

### DETAILED DESCRIPTION OF INVENTION

The instant invention satisfies the foregoing needs and fulfills additional objects and advantages by providing novel pharmaceutical compositions that include the novel use of newly discovered tight junction-opening peptides to enhance mucosal delivery of the biologically active agent in a mammalian subject.

One aspect of the invention is a pharmaceutical formulation comprising a biologically active agent and a mucosal delivery-enhancing effective amount of a tight junction modulating peptide (TJMP) that reversibly enhances mucosal epithelial transport of a biologically active agent in a mammalian subject.

Preferably, a tight junction modulator component contains a peptide or protein portion consisting of 2-500 amino acid residues, or 2-100 amino acid residues, or 2-50 amino acid residues. The tight junction modulator peptide or protein may be monomeric or oligomeric, for example, dimeric.

The tight junction modulating peptide can be produced by recombinant or chemical synthesis means, consistent with techniques known to those skilled in the appropriate art.

Peptides capable modulating the function of epithelial tight junctions have been previously described (Johnson, P. H. and S. C. Quay, Expert. Opin. Drug Deliv. 2:281-98, 2000). In particular, a novel tight junction modulating (TJM) peptide, PN159, was shown to reduce transepithelial electrical resistance (TER) across a tissue barrier and increase paracellular transport of 3,000 Da MW dextran with low cytotoxicity and high retention of cell viability.

In preferred embodiments of the invention, the TJMP is selected from the group consisting of:

NH2-KLALKLALKALKAALKLA-amide
NH2-GWTLNSAGYLLGKINLKALAALAKKIL-amide
NH2-LLETLLKPFQCRICMRNFSTRQARRNHRRRHRR-amide
NH2-AAVALLPAVLLALLAPRKKRRQRRRPPQ-amide
NH2-RKKRRQRRRPPQCAAVALLPAVLLALLAP-amide
NH2-RQIKIWFQNRRMKWKK-amide
NH2-KGSKKAVTKAQKKDGKKRKRSRKESYSVYVYKVLKQ-amide
NH2-KLWSAWPSLWSSLWKP-amide
NH2-RRRQRRKRGGDIMGEWGNEIFGAIAGFLG-amide
Maleimide-GLGSLLKKAGKKLKQPKSKRKV-amide
NH2-KETWWETWWTEWSQPGPKKRRQRRRRPPQ-amide.

In other preferred embodiments of the invention, the TJMP is selected from the group consisting of:

CNGRCGGKKKLKLLLKLL
LRKLRKRLLRLRKLRKRLLR.

In one aspect, this invention describes formulations of therapeutic small molecules, peptide and proteins that are suitable for transmucosal delivery, wherein transmucosal delivery is facilitated by the presence of a tight junction modulator peptide, wherein said peptide is conjugated a water soluble polymer. Preferably, the water soluble polymer is a polyalkylene oxide selected from the group consisting of alpha-substituted polyalkylene oxide derivatives, alkyl-capped polyethylene oxides, bis-polyethylene oxides, poly(orthoesters) such as poly(lactic-co-glycolide) and derivatives thereof, polyethylene glycol (PEG) homopolymers and derivatives thereof, polypropylene glycol homopolymers and derivatives thereof, copolymers of poly(alkylene oxides), and block copolymers of poly(alkylene oxides) or activated derivatives thereof. Preferably, the polyalkylene oxide has a molecular weight of about 200 to about 50,000. More preferably, the polyalkylene oxide has a molecular weight of about 200 to about 20,000. Especially preferred polyalkylene oxides are polyethylene glycol and polyethylene oxide.

The TJMP may be conjugated to more than one water soluble chain. In a preferred embodiment the poly(alkylene oxide) chain is a polyethylene glycol (PEG) chain, which may have a molecular size between about 0.2 and about 200 kiloDaltons (kDa).

The water-soluble polymer may be conjugated to the tight junction modulator peptide via a spacer. This linkage may be reversible. The water-soluble polymer may be linear or may be branched.

In one embodiment, the peptide is covalently linked to a single poly(alkylene oxide) chain. In a related embodiment, the poly(alkylene oxide) has a polydispersity value (Mw/Mn) of less than 2.00, or less than 1.20. The poly(alkylene oxide) chain may be branched or unbranched.

Conjugation with water-soluble polymers such as poly(ethylene glycol) (PEG) and derivatives of PEG have been used as a strategy to enhance the half life of proteins, in particular for injected dosage forms (Caliceti, P. and F. M. Veronese, Adv. Drug Deliv. Rev. 55:1261-77, 2003). Other potential benefits of modification of peptides and proteins with polymers such as PEG include chemical (Diwan, M. and T. G. Park, Int. J. Pharm. 252:111-22, 2003) and biochemical stabilization (Na, D. H., et al., J. Pharm. Sci. 93:256-61, 2004) and attenuation of immunogenicity (Yang, Z., et al., Cancer Res. 64:6673-78, 2004).

Most examples for use of PEG conjugated to proteins is where the PEG chain has a molecular weight of sufficient length to impart the effect described above. In particular, it has been described that at least a 20 kDa MW PEG is required. For example, Holtsberg et al. (Holtsberg, F. W., et al., J. Control Rel. 80:259-71, 2002) showed that for the protein arginine deiminase conjugated to PEG, when PEG was 20 kDa or greater there was an increase in pharmacokinetic and pharmacodynamic properties of the formulation when administered intravenously in mice. When PEG MW was lower than 20 kDa, there was little effect. In another example, mono-PEGylation to the peptide salmon calcitonin results in increased intranasal bioavailability in rats, with the enhancement being inversely proportional to the PEG molecular weight (MW) (Lee, K. C. et. al., Calcif. Tissue Int. 73:545-9, 2003, and Shin, B. S., et al., Chem. Pharm. Bull. (Tokyo) 52:957-60, 2004), hereby incorporated by reference in their entirety.

Some preferred poly(alkylene oxides) are selected from the group consisting of alpha-substituted poly(alkylene oxide) derivatives, poly(ethylene glycol) (PEG) homopolymers and derivatives thereof, poly(propylene glycol) (PPG) homopolymers and derivatives thereof, poly(ethylene oxides) (PEO) polymers and derivatives thereof, bis-poly(ethylene oxides) and derivatives thereof, copolymers of poly(alkylene oxides), and block copolymers of poly(alkylene oxides), poly(lactide-co-glycolide) and derivatives thereof, or activated derivatives thereof. The water-soluble polymer may have a molecular weight of about 200 to about 40000 Da, preferably about 200 to about 20000 Da, or about 200 to 10000 Da, or about 200 to 5000 Da.

The conjugate between the tight junction modulating peptide and the PEG or other water soluble polymer may be resistant to physiological processes, including proteolysis, enzyme action or hydrolysis in general. Alternatively, the conjugate can be cleaved by processes of biodegradation, for example a pro-drug approach. Preferably, the molecule is covalently linked to a single poly(alkylene oxide) chain, which may be unbranched or branched. The means of conjugation are generally known to ordinary skilled workers, for examples, U.S. Pat. No. 5,595,732; U.S. Pat. No. 5,766,897; U.S. Pat. No. 5,985,265; U.S. Pat. No. 6,528,485; U.S. Pat. No. 6,586,398; U.S. Pat. No. 6,869,932; and U.S. Pat. No. 6,706,289.

In another aspect of the invention, the TJMP decreases electrical resistance across a mucosal tissue barrier. In a preferred embodiment, the decrease in electrical resistance is at least 80% of the electrical resistance prior to applying the enhancer of permeation. In a related embodiment, the TJMP increases permeability of the molecule across a mucosal tissue barrier, preferably at least two fold. In another embodiment, the increased permeability is paracellular. In another embodiment, the increased permeability results from modification of tight junctions. In an alternate embodiment, the increased permeability is transcellular, or a combination of trans- and paracellular.

In another aspect of the invention the mucosal tissue layer is comprised of an epithelial cell layer. In a preferred embodiment, the epithelial cell is selected from the group consisting of tracheal, bronchial, alveolar, nasal, pulmonary, gastrointestinal, epidermal or buccal, preferably nasal.

In another aspect of the invention an active agent is a peptide or protein. The peptide or protein may have between 2 and 1000 amino acids. In a preferred embodiment, the peptide or protein is comprised of between 2 and 50 amino acids. In another embodiment, the peptide or protein is cyclic. In another embodiment, the peptide or protein forms dimers or higher-order oligomers via physical or chemical bonding.

In a preferred embodiment, the peptide or protein is selected from the group comprising GLP-1, PYY₃₋₃₆, PTH₁₋₃₄ and Exendin-4. In another embodiment, the biologically active agent is a protein, preferably selected from the group consisting of beta-interferon, alpha-interferon, insulin, erythropoietin, G-CSF, and GM-CSF, growth hormone, and analogues of any of these.

The permeabilizing peptides of the invention include PN529, containing the sequence WEAALAEALAEALAEHLASQPKSKRKV (SEQ ID NO 57).

Another aspect of the invention is a method of administering a molecule to an animal comprising preparing any of the formulations above, and bringing such formulation in contact with a mucosal surface of such animal. In a preferred embodiment, the mucosal surface is intranasal.

Another aspect of the invention is a dosage form comprising any of the formulations above, in which the dosage form is liquid, preferably in the form of droplets. Alternatively, the dosage form may be solid, either, to be reconstituted in liquid prior to administration, to be administered as a powder, or in the form of a capsule, tablet or gel.

Another aspect of the invention is a molecule that reversibly enhances mucosal epithelial transport of a biological agent in a mammalian subject, having a tight junction modulating component peptide (TJMP), a TJMP analogue, a conjugate of a TJMP or a TJMP analogue, or complexes thereof.

The permeabilizing peptides of the invention include PN159, having the sequence NH2-KLALKLALKALKAALKLA-amide. Included in the invention are analogues of PN159 as disclosed herein, combinations of those analogs, and any derivatives, variants, fragments, mimetics, or fusion molecules of PN159.

The permeabilizing agent reversibly enhances mucosal epithelial paracellular transport, typically by modulating epithelial tight junction structures and/or physiology at a mucosal epithelial surface in the subject. This effect typically involves inhibition by the permeabilizing agent of homotypic or heterotypic binding between epithelial membrane adhesive proteins of neighboring epithelial cells. Target proteins for this blockade of homotypic or heterotypic binding can be selected from various related junctional adhesion molecules (JAMs), occludins, or claudins.

### Epithelial Cell Biology

A cDNA encoding murine junctional adhesion molecule-1 (JAM-1) has been cloned and corresponds to a predicted type I transmembrane protein (comprising a single transmembrane domain) with a molecular weight of approximately 32-kD (Williams, et al., Molecular Immunology 36:1175-1188, 1999; Gupta, et al., IUBMB Life 50:51-56, 2000; Ozaki, et al., J. Immunol. 163:553-557, 1999; Martin-Padura, et al., J. Cell. Biol. 142:117-127, 1998). The extracellular segment of the molecule comprises two Ig-like domains described as an amino terminal “VH-type” and a carboxy-terminal “C2-type” carboxy-terminal β-sandwich fold (Bazzoni et al., Microcirculation 8:143-152, 2001). Murine JAM-1 also contains two sites for N-glycosylation, and a cytoplasmic domain. The JAM-1 protein is a member of the immunoglobulin (Ig) superfamily and localizes to tight junctions of both epithelial and endothelial cells. Ultrastructural studies indicate that JAM-1 is limited to the membrane regions containing fibrils of occludin and claudin.

Another JAM family member, designated “Vascular endothelial junction-associated molecule” (VE-JAM), contains two extracellular immunoglobulin-like domains, a transmembrane domain, and a relatively short cytoplasmic tail. VE-JAM is principally localized to intercellular boundaries of endothelial cells (Palmeri, et al., J. Biol. Chem. 275:19139-19145, 2000). VE-JAM is highly expressed highly by endothelial cells of venules, and is also expressed by endothelia of other vessels. Another reported JAM family member, JAM-3, has a predicted amino acid sequence that displays 36% and 32% identity, respectively, to JAM-2 and JAM-1. JAM-3 shows widespread tissue expression with higher levels apparent in the kidney, brain, and placenta. At the cellular level, JAM-3 transcript is expressed within endothelial cells. JAM-3 and JAM-2 have been reported to be binding partners. In particular, the JAM-3 ectodomain reportedly binds to JAM2-Fc. JAM-3 protein is up-regulated on peripheral blood lymphocytes following activation. (Pia Arrate, et al., J. Biol. Chem. 276:45826-45832, 2001).

Another proposed trans-membrane adhesive protein involved in epithelial tight junction regulation is Occludin. Occludin is an approximately 65-kD type II transmembrane protein composed of four transmembrane domains, two extracellular loops, and a large C-terminal cytosolic domain (Furuse, et al., J. Cell. Biol. 123:1777-1788 (1993); Furuse, et al., J. Cell. Biol. 127:1617-1626, 1994). When observed by immuno-freeze fracture electron microscopy, occludin is concentrated directly within the tight junction fibrils (Fujimoto, J. Cell. Sci. 108:3443-3449, 1995).

Two additional integral membrane proteins of the tight junction, claudin-1 and claudin-2, were identified by direct biochemical fractionation of junction-enriched membranes from chicken liver (Furuse, et al., J. Cell. Biol. 141:1539-1550, 1998). Claudin-1 and claudin-2 were found to copurify with occludin as a broad approximately 22-kD gel band on sodium dodecyl sulfate-polyacrylamide gel electrophoresis. The deduced sequences of two closely related proteins cloned from a mouse cDNA library predict four transmembrane helices, two short extracellular loops, and short cytoplasmic N- and C-termini. Despite topologies similar to that of occludin, they share no sequence homology. Subsequently, six more claudin gene products (claudin-3 through claudin-8) have been cloned and have been shown to localize within tight junction fibrils, as determined by immunogold freeze fracture labeling (Morita, et al., Proc. Natl. Acad. Sci. USA 96:511-516, 1999). Given that a barrier remains in the absence of occludin, claudin-1 through claudin-8 have been considered as candidates for the primary seal-forming elements of the extracellular space.

Other cytoplasmic proteins that have been localized to epithelial junctions include zonulin, symplekin, cingulin, and 7H6. Zonulins reportedly are cytoplasmic proteins that bind the cytoplasmic tail of occludin. Representing this family of proteins are “ZO-1, ZO-2, and ZO-3”. Zonulin is postulated to be a human protein analogue of the Vibrio cholerae derived zonula occludens toxin (ZOT).

Zonulin likely plays a role in tight junction regulation during developmental, physiological, and pathological processes—including tissue morphogenesis, movement of fluid, macromolecules and leukocytes between the intestinal lumen and the interstitium, and inflammatory/autoimmune disorders (see, e.g., Wang, et al., J. Cell. Sci. 113:4435-40, 2000; Fasano, et al., Lancet 355:1518-9, 2000; Fasano, Ann. N.Y. Acad. Sci., 915:214-222, 2000). Zonulin expression increased in intestinal tissues during the acute phase of coeliac disease, a clinical condition in which tight junctions are opened and permeability is increased. Zonulin induces tight junction disassembly and a subsequent increase in intestinal permeability in non-human primate intestinal epithelia in vitro.

Comparison of amino acids in the active V. cholerae ZOT fragment and human zonulin identified a putative receptor binding domain within the N-terminal region of the two proteins. The ZOT biologically active domain increases intestinal permeability by interacting with a mammalian cell receptor with subsequent activation of intracellular signaling leading to the disassembly of the intercellular tight junction. The ZOT biologically active domain has been localized toward the carboxyl terminus of the protein and coincides with the predicted cleavage product generated by V. cholerae. This domain shares a putative receptor-binding motif with zonulin, the ZOT mammalian analogue. Amino acid comparison between the ZOT active fragment and zonulin, combined with site-directed mutagenesis experiments, suggest an octapeptide receptor-binding domain toward the amino terminus of processed ZOT and the amino terminus of zonulin. (Di Pierro, et al., J. Biol. Chem. 276:19160-19165, 2001). ZO-1 reportedly binds actin, AF-6, ZO-associated kinase (ZAK), fodrin, and α-catenin.

Permeabilizing peptides for use within the invention include natural or synthetic, therapeutically or prophylactically active, peptides (comprised of two or more covalently linked amino acids), proteins, peptide or protein fragments, peptide or protein analogs, peptide or protein mimetics, and chemically modified derivatives or salts of active peptides or proteins. Thus, as used herein, the term “permeabilizing peptide” will often be intended to embrace all of these active species, i.e., peptides and proteins, peptide and protein fragments, peptide and protein analogs, peptide and protein mimetics, and chemically modified derivatives and salts of active peptides or proteins. Often, the permeabilizing peptides or proteins are muteins that are readily obtainable by partial substitution, addition, or deletion of amino acids within a naturally occurring or native (e.g., wild-type, naturally occurring mutant, or allelic variant) peptide or protein sequence. Additionally, biologically active fragments of native peptides or proteins are included. Such mutant derivatives and fragments substantially retain the desired biological activity of the native peptide or proteins. In the case of peptides or proteins having carbohydrate chains, biologically active variants marked by alterations in these carbohydrate species are also included within the invention.

The permeabilizing peptides, proteins, analogs and mimetics for use within the methods and compositions of the invention are often formulated in a pharmaceutical composition comprising a mucosal delivery-enhancing or permeabilizing effective amount of the permeabilizing peptide, protein, analog or mimetic that reversibly enhances mucosal epithelial paracellular transport by modulating epithelial junctional structure and/or physiology in a mammalian subject.

### Biologically Active Agents

The methods and compositions of the present invention are directed toward enhancing mucosal, e.g., intranasal, delivery of a broad spectrum of biologically active agents to achieve therapeutic, prophylactic or other desired physiological results in mammalian subjects. As used herein, the term “biologically active agent” encompasses any substance that produces a physiological response when mucosally administered to a mammalian subject according to the methods and compositions herein. Useful biologically active agents in this context include therapeutic or prophylactic agents applied in all major fields of clinical medicine, as well as nutrients, cofactors, enzymes (endogenous or foreign), antioxidants, and the like. Thus, the biologically active agent may be water-soluble or water-insoluble, and may include higher molecular weight proteins, peptides, carbohydrates, glycoproteins, lipids, and/or glycolipids, nucleosides, polynucleotides, and other active agents.

Useful pharmaceutical agents within the methods and compositions of the invention include drugs and macromolecular therapeutic or prophylactic agents embracing a wide spectrum of compounds, including small molecule drugs, peptides, proteins, and vaccine agents. Exemplary pharmaceutical agents for use within the invention are biologically active for treatment or prophylaxis of a selected disease or condition in the subject. Biological activity in this context can be determined as any significant (i.e., measurable, statistically significant) effect on a physiological parameter, marker, or clinical symptom associated with a subject disease or condition, as evaluated by an appropriate in vitro or in vivo assay system involving actual patients, cell cultures, sample assays, or acceptable animal models.

The methods and compositions of the invention provide unexpected advantages for treatment of diseases and other conditions in mammalian subjects, which advantages are mediated, for example, by providing enhanced speed, duration, fidelity or control of mucosal delivery of therapeutic and prophylactic compounds to reach selected physiological compartments in the subject (e.g., into or across the nasal mucosa, into the systemic circulation or central nervous system (CNS), or to any selected target organ, tissue, fluid or cellular or extracellular compartment within the subject).

In various exemplary embodiments, the methods and compositions of the invention may incorporate one or more biologically active agent(s) selected from:

opiods or opiod antagonists, such as morphine, hydromorphone, oxymorphone, lovorphanol, levallorphan, codeine, nalmefene, nalorphine, nalozone, naltrexone, buprenorphine, butorphanol, and nalbufine;

corticosterones, such as cortisone, hydrocortisone, fludrocortisone, prednisone, prednisolone, methylprednisolone, triamcinolone, dexamethoasone, betamethoasone, paramethosone, and fluocinolone;

other anti-inflammatories, such as colchicine, ibuprofen, indomethacin, and piroxicam; anti-viral agents such as acyclovir, ribavarin, trifluorothyridine, Ara-A (Arabinofuranosyladenine), acylguanosine, nordeoxyguanosine, azidothymidine, dideoxyadenosine, and dideoxycytidine; antiandrogens such as spironolactone;

androgens, such as testosterone;

estrogens, such as estradiol;

progestins;

muscle relaxants, such as papaverine;

vasodilators, such as nitroglycerin, vasoactive intestinal peptide and calcitonin related gene peptide;

antihistamines, such as cyproheptadine;

agents with histamine receptor site blocking activity, such as doxepin, imipramine, and cimetidine;

antitussives, such as dextromethorphan; neuroleptics such as clozaril; antiarrhythmics;

antiepileptics,

enzymes, such as superoxide dismutase and neuroenkephalinase;

anti-fungal agents, such as amphotericin B, griseofulvin, miconazole, ketoconazole, tioconazol, itraconazole, and fluconazole;

antibacterials, such as penicillins, cephalosporins, tetracyclines, aminoglucosides, erythromycin, gentamicins, polymyxin B;

anti-cancer agents, such as 5-fluorouracil, bleomycin, methotrexate, and hydroxyurea, dideoxyinosine, floxuridine, 6-mercaptopurine, doxorubicin, daunorubicin, I-darubicin, taxol and paclitaxel;

antioxidants, such as tocopherols, retinoids, carotenoids, ubiquinones, metal chelators, and phytic acid;

antiarrhythmic agents, such as quinidine; and

antihypertensive agents such as prazosin, verapamil, nifedipine, and diltiazem; analgesics such as acetaminophen and aspirin;

monoclonal and polyclonal antibodies, including humanized antibodies, and antibody fragments;

anti-sense oligonucleotides; and

RNA, DNA and viral vectors comprising genes encoding therapeutic peptides and proteins.

In addition to these exemplary classes and species of active agents, the methods and compositions of the invention embrace any physiologically active agent, as well as any combination of multiple active agents, described above or elsewhere herein or otherwise known in the art, that is individually or combinatorially effective within the methods and compositions of the invention for treatment or prevention of a selected disease or condition in a mammalian subject (see, Physicians' Desk Reference, published by Medical Economics Company, a division of Litton Industries, Inc).

Regardless of the class of compound employed, the biologically active agent for use within the invention will be present in the compositions and methods of the invention in an amount sufficient to provide the desired physiological effect with no significant, unacceptable toxicity or other adverse side effects to the subject. The appropriate dosage levels of all biologically active agents will be readily determined without undue experimentation by the skilled artisan. Because the methods and compositions of the invention provide for enhanced delivery of the biologically active agent(s), dosage levels significantly lower than conventional dosage levels may be used with success. In general, the active substance will be present in the composition in an amount of from about 0.01% to about 50%, often between about 0.1% to about 20%, and commonly between about 1.0% to 5% or 10% by weight of the total intranasal formulation depending upon the particular substance employed.

As used herein, the terms biologically active “peptide” and “protein” include polypeptides of various sizes, and do not limit the invention to amino acid polymers of any particular size. Peptides from as small as a few amino acids in length, to proteins of any size, as well as peptide-peptide, protein-protein fusions and protein-peptide fusions, are encompassed by the present invention, so long as the protein or peptide is biologically active in the context of eliciting a specific physiological, immunological, therapeutic, or prophylactic effect or response.

The instant invention provides novel formulations and coordinate administration methods for enhanced mucosal delivery of biologically active peptides and proteins. Illustrative examples of therapeutic peptides and proteins for use within the invention include, but are not limited to: tissue plasminogen activator (TPA), epidermal growth factor (EGF), fibroblast growth factor (FGF-acidic or basic), platelet derived growth factor (PDGF), transforming growth factor (TGF-alpha or beta), vasoactive intestinal peptide, tumor necrosis factor (TNF), hypothalmic releasing factors, prolactin, thyroid stimulating hormone (TSH), adrenocorticotropic hormone (ACTH), parathyroid hormone (PTH), follicle stimulating hormone (FSF), luteinizing hormone releasing hormone (LHRH), endorphins, glucagon, calcitonin, oxytocin, carbetocin, aldoetecone, enkaphalins, somatostin, somatotropin, somatomedin, gonadotrophin, estrogen, progesterone, testosterone, alpha-melanocyte stimulating hormone, non-naturally occurring opiods, lidocaine, ketoprofen, sufentainil, terbutaline, droperidol, scopolamine, gonadorelin, ciclopirox, buspirone, calcitonin, cromolyn sodium or midazolam, cyclosporin, lisinopril, captopril, delapril, cimetidine, ranitidine, famotidine, superoxide dismutase, asparaginase, arginase, arginine deaminease, adenosine deaminase ribonuclease, trypsin, chemotrypsin, and papain. Additional examples of useful peptides include, but are not limited to, bombesin, substance P, vasopressin, alpha-globulins, transferrin, fibrinogen, beta-lipoproteins, beta-globulins, prothrombin, ceruloplasmin, alpha₂-glycoproteins, alpha₂-globulins, fetuin, alpha₁-lipoproteins, alpha₁-globulins, albumin, prealbumin, and other bioactive proteins and recombinant protein products.

In more detailed aspects of the invention, methods and compositions are provided for enhanced mucosal delivery of specific, biologically active peptide or protein therapeutics to treat (i.e., to eliminate, or reduce the occurrence or severity of symptoms of) an existing disease or condition, or to prevent onset of a disease or condition in a subject identified to be at risk for the subject disease or condition. Biologically active peptides and proteins that are useful within these aspects of the invention include, but are not limited to hematopoietics; antiinfective agents; antidementia agents; antiviral agents; antitumoral agents; antipyretics; analgesics; antiinflammatory agents; antiulcer agents; antiallergic agents; antidepressants; psychotropic agents; cardiotonic, antiarrythmic agents; vasodilators; antihypertensive agents such as hypotensive diuretics; antidiabetic agents; anticoagulants; cholesterol lowering agents; therapeutic agents for osteoporosis; hormones; antibiotics; vaccines; and the like.

Biologically active peptides and proteins for use within these aspects of the invention include, but are not limited to, cytokines; peptide hormones; growth factors; factors acting on the cardiovascular system; cell adhesion factors; factors acting on the central and peripheral nervous systems; factors acting on humoral electrolytes and hemal organic substances; factors acting on bone and skeleton growth or physiology; factors acting on the gastrointestinal system; factors acting on the kidney and urinary organs; factors acting on the connective tissue and skin; factors acting on the sense organs; factors acting on the immune system; factors acting on the respiratory system; factors acting on the genital organs; and various enzymes.

For example, hormones which may be administered within the methods and compositions of the present invention include androgens, estrogens, prostaglandins, somatotropins, gonadotropins, interleukins, steroids and cytokines.

Vaccines which may be administered within the methods and compositions of the present invention include bacterial and viral vaccines, such as vaccines for hepatitis, influenza, respiratory syncytial virus (RSV), parainfluenza virus (PIV), tuberculosis, canary pox, chicken pox, measles, mumps, rubella, pneumonia, and human immunodeficiency virus (HIV).

Bacterial toxoids which may be administered within the methods and compositions of the present invention include diphtheria, tetanus, pseudonomas and mycobacterium tuberculosis.

Examples of specific cardiovascular or thromobolytic agents for use within the invention include hirugen, hirulos and hirudine.

Antibody reagents that are usefully administered with the present invention include monoclonal antibodies, polyclonal antibodies, humanized antibodies, antibody fragments, fusions and multimers, and immunoglobins.

As used herein, the term “conservative amino acid substitution” refers to the general interchangeability of amino acid residues having similar side chains. For example, a commonly interchangeable group of amino acids having aliphatic side chains is alanine, valine, leucine, and isoleucine; a group of amino acids having aliphatic-hydroxyl side chains is serine and threonine; a group of amino acids having amide-containing side chains is asparagine and glutamine; a group of amino acids having aromatic side chains is phenylalanine, tyrosine, and tryptophan; a group of amino acids having basic side chains is lysine, arginine, and histidine; and a group of amino acids having sulfur-containing side chains is cysteine and methionine. Examples of conservative substitutions include the substitution of a non-polar (hydrophobic) residue such as isoleucine, valine, leucine or methionine for another. Likewise, the present invention contemplates the substitution of a polar (hydrophilic) residue such as between arginine and lysine, between glutamine and asparagine, and between threonine and serine. Additionally, the substitution of a basic residue such as lysine, arginine or histidine for another or the substitution of an acidic residue such as aspartic acid or glutamic acid for another is also contemplated. Exemplary conservative amino acids substitution groups are: valine-leucine-isoleucine, phenylalanine-tyrosine, lysine-arginine, alanine-valine, and asparagine-glutamine.

The term biologically active peptide or protein analog further includes modified forms of a native peptide or protein incorporating stereoisomers (e.g., D-amino acids) of the twenty conventional amino acids, or unnatural amino acids such as α,α-disubstituted amino acids, N-alkyl amino acids, lactic acid. These and other unconventional amino acids may also be substituted or inserted within native peptides and proteins useful within the invention. Examples of unconventional amino acids include: 4-hydroxyproline, γ-carboxyglutamate, ε-N,N,N-trimethyllysine, ε-N-acetyllysine, O-phosphoserine, N-acetylserine, N-formylmethionine, 3-methylhistidine, 5-hydroxylysine, ω-N-methylarginine, and other similar amino acids and imino acids (e.g., 4-hydroxyproline). In addition, biologically active peptide or protein analogs include single or multiple substitutions, deletions and/or additions of carbohydrate, lipid and/or proteinaceous moieties that occur naturally or artificially as structural components of the subject peptide or protein, or are bound to or otherwise associated with the peptide or protein.

In one aspect, peptides (including polypeptides) useful within the invention are modified to produce peptide mimetics by replacement of one or more naturally occurring side chains of the 20 genetically encoded amino acids (or D amino acids) with other side chains, for instance with groups such as alkyl, lower alkyl, cyclic 4-, 5-, 6-, to 7-membered alkyl, amide, amide lower alkyl, amide di(lower alkyl), lower alkoxy, hydroxy, carboxy and the lower ester derivatives thereof, and with 4-, 5-, 6-, to 7-membered heterocyclics. For example, proline analogs can be made in which the ring size of the proline residue is changed from 5 members to 4, 6, or 7 members. Cyclic groups can be saturated or unsaturated, and if unsaturated, can be aromatic or non-aromatic. Heterocyclic groups can contain one or more nitrogen, oxygen, and/or sulphur heteroatoms. Examples of such groups include the furazanyl, furyl, imidazolidinyl, imidazolyl, imidazolinyl, isothiazolyl, isoxazolyl, morpholinyl (e.g., morpholino), oxazolyl, piperazinyl (e.g., 1-piperazinyl), piperidyl (e.g., 1-piperidyl, piperidino), pyranyl, pyrazinyl, pyrazolidinyl, pyrazolinyl, pyrazolyl, pyridazinyl, pyridyl, pyrimidinyl, pyrrolidinyl (e.g., 1-pyrrolidinyl), pyrrolinyl, pyrrolyl, thiadiazolyl, thiazolyl, thienyl, thiomorpholinyl (e.g., thiomorpholino), and triazolyl. These heterocyclic groups can be substituted or unsubstituted. Where a group is substituted, the substituent can be alkyl, alkoxy, halogen, oxygen, or substituted or unsubstituted phenyl.

Peptides and proteins, as peptide and protein analogs and mimetics, can also be covalently bound to one or more of a variety of nonproteinaceous polymers, e.g., polyethylene glycol, polypropylene glycol, or polyoxyalkenes, in the manner set forth in U.S. Pat. No. 4,640,835; U.S. Pat. No. 4,496,689; U.S. Pat. No. 4,301,144; U.S. Pat. No. 4,670,417; U.S. Pat. No. 4,791,192; or U.S. Pat. No. 4,179,337.

Other peptide and protein analogs and mimetics within the invention include glycosylation variants, and covalent or aggregate conjugates with other chemical moieties. Covalent derivatives can be prepared by linkage of functionalities to groups which are found in amino acid side chains or at the N- or C-termini, by means which are well known in the art. These derivatives can include, without limitation, aliphatic esters or amides of the carboxyl terminus, or of residues containing carboxyl side chains, O-acyl derivatives of hydroxyl group-containing residues, and N-acyl derivatives of the amino terminal amino acid or amino-group containing residues, e.g., lysine or arginine. Acyl groups are selected from the group of alkyl-moieties including C3 to C18 normal alkyl, thereby forming alkanoyl aroyl species. Covalent attachment to carrier proteins, e.g., immunogenic moieties may also be employed.

In addition to these modifications, glycosylation alterations of biologically active peptides and proteins can be made, e.g., by modifying the glycosylation patterns of a peptide during its synthesis and processing, or in further processing steps. Particularly preferred means for accomplishing this are by exposing the peptide to glycosylating enzymes derived from cells that normally provide such processing, e.g., mammalian glycosylation enzymes. Deglycosylation enzymes can also be successfully employed to yield useful modified peptides and proteins within the invention. Also embraced are versions of a native primary amino acid sequence which have other minor modifications, including phosphorylated amino acid residues, e.g., phosphotyrosine, phosphoserine, or phosphothreonine, or other moieties, including ribosyl groups or cross-linking reagents.

Peptidomimetics may also have amino acid residues that have been chemically modified by phosphorylation, sulfonation, biotinylation, or the addition or removal of other moieties, particularly those that have molecular shapes similar to phosphate groups.

One can cyclize active peptides for use within the invention, or incorporate a desamino or descarboxy residue at the termini of the peptide, so that there is no terminal amino or carboxyl group, to decrease susceptibility to proteases, or to restrict the conformation of the peptide. C-terminal functional groups among peptide analogs and mimetics of the present invention include amide, amide lower alkyl, amide di(lower alkyl), lower alkoxy, hydroxy, and carboxy, and the lower ester derivatives thereof, and the pharmaceutically acceptable salts thereof.

A variety of additives, diluents, bases and delivery vehicles are provided within the invention that effectively control water content to enhance protein stability. These reagents and carrier materials effective as anti-aggregation agents in this sense include, for example, polymers of various functionalities, such as polyethylene glycol, dextran, diethylaminoethyl dextran, and carboxymethyl cellulose, which significantly increase the stability and reduce the solid-phase aggregation of peptides and proteins admixed therewith or linked thereto. In some instances, the activity or physical stability of proteins can also be enhanced by various additives to aqueous solutions of the peptide or protein drugs. For example, additives, such as polyols (including sugars), amino acids, proteins such as collagen and gelatin, and various salts may be used.

Certain additives, in particular sugars and other polyols, also impart significant physical stability to dry, e.g., lyophilized proteins. These additives can also be used within the invention to protect the proteins against aggregation not only during lyophilization but also during storage in the dry state. For example sucrose and Ficoll 70 (a polymer with sucrose units) exhibit significant protection against peptide or protein aggregation during solid-phase incubation under various conditions. These additives may also enhance the stability of solid proteins embedded within polymer matrices.

Yet additional additives, for example sucrose, stabilize proteins against solid-state aggregation in humid atmospheres at elevated temperatures, as may occur in certain sustained-release formulations of the invention. Proteins such as gelatin and collagen also serve as stabilizing or bulking agents to reduce denaturation and aggregation of unstable proteins in this context. These additives can be incorporated into polymeric melt processes and compositions within the invention. For example, polypeptide microparticles can be prepared by simply lyophilizing or spray drying a solution containing various stabilizing additives described above. Sustained release of unaggregated peptides and proteins can thereby be obtained over an extended period of time.

Various additional preparative components and methods, as well as specific formulation additives, are provided herein which yield formulations for mucosal delivery of aggregation-prone peptides and proteins, wherein the peptide or protein is stabilized in a substantially pure, unaggregated form. A range of components and additives are contemplated for use within these methods and formulations. Exemplary of these anti-aggregation agents are linked dimers of cyclodextrins (CDs), which selectively bind hydrophobic side chains of polypeptides. These CD dimers have been found to bind to hydrophobic patches of proteins in a manner that significantly inhibits aggregation. This inhibition is selective with respect to both the CD dimer and the protein involved. Such selective inhibition of protein aggregation provides additional advantages within the intranasal delivery methods and compositions of the invention. Additional agents for use in this context include CD trimers and tetramers with varying geometries controlled by the linkers that specifically block aggregation of peptides and proteins (Breslow et al., J. Am. Chem. Soc. 118:11678-11681, 1996; Breslow et al., PNAS USA 94:11156-11158, 1997).

### Charge Modifying and pH Control Agents and Methods

To improve the transport characteristics of biologically active agents (e.g., macromolecular drugs, peptides or proteins) for enhanced delivery across hydrophobic mucosal membrane barriers, the invention also provides techniques and reagents for charge modification of selected biologically active agents or delivery-enhancing agents described herein. In this regard, the relative permeabilities of macromolecules is generally be related to their partition coefficients. The degree of ionization of molecules, which is dependent on the pKₐ of the molecule and the pH at the mucosal membrane surface, also affects permeability of the molecules. Permeation and partitioning of biologically active agents and permeabilizing agents for mucosal delivery may be facilitated by charge alteration or charge spreading of the active agent or permeabilizing agent, which is achieved, for example, by alteration of charged functional groups, by modifying the pH of the delivery vehicle or solution in which the active agent is delivered, or by coordinate administration of a charge- or pH-altering reagent with the active agent.

### Preservatives

Preservative such as chlorobutanol, methyl paraben, propyl paraben, sodium benzoate (0.5%), phenol, cresol, p-chloro-m-cresol, phenylethyl alcohol, benzyl alcohol, phenylmercuric acetate, phenylmercuric borate, phenylmercuric nitrate, thimerosal, sorbic acid, benzethonium chloride or benzylkonium chloride can be added to the formulations of the invention to inhibit microbial growth.

### pH and Buffering Systems

The pH is generally regulated using a buffer such as a system comprised of citric acid and a citrate salt(s), such as sodium citrate. Additional suitable buffer systems include acetic acid and an acetate salt system, succinic acid and a succinate salt system, malic acid and a malic salt system, and gluconic acid and a gluconate salt system. Alternatively, buffer systems comprised of mixed acid/salt systems can be employed, such as an acetic acid and sodium citrate system, a citrate acid, sodium acetate system, and a citric acid, sodium citrate, sodium benzoate system. For any buffer system, additional acids, such as hydrochloric acid, and additional bases, such as sodium hydroxide, may be added for final pH adjustment.

Additional Agents for Modulating Epithelial Junction Structure and/or Physiology

Epithelial tight junctions are generally impermeable to molecules with radii of approximately 15 angstroms, unless treated with junctional physiological control agents that stimulate substantial junctional opening as provided within the instant invention. Among the “secondary” tight junctional regulatory components that will serve as useful targets for secondary physiological modulation within the methods and compositions of the invention, the ZO1-ZO2 heterodimeric complex has shown itself amenable to physiological regulation by exogenous agents that can readily and effectively alter paracellular permeability in mucosal epithelia. On such agent that has been extensively studied is the bacterial toxin from Vibrio cholerae known as the “zonula occludens toxin” (ZOT). See also, WO 96/37196; U.S. Pat. Nos. 5,945,510; 5,948,629; 5,912,323; 5,864,014; 5,827,534; 5,665,389; and 5,908,825. Thus, ZOT and other agents that modulate the ZO1-ZO2 complex will be combinatorially formulated or coordinately administered with one or more biologically active agents.

### Formulation and Administration

Mucosal delivery formulations of the present invention comprise the biologically active agent to be administered typically combined together with one or more pharmaceutically acceptable carriers and, optionally, other therapeutic ingredients. The carrier(s) must be “pharmaceutically acceptable” in the sense of being compatible with the other ingredients of the formulation and not eliciting an unacceptable deleterious effect in the subject. Such carriers are described herein above or are otherwise well known to those skilled in the art of pharmacology. Desirably, the formulation should not include substances such as enzymes or oxidizing agents with which the biologically active agent to be administered is known to be incompatible. The formulations may be prepared by any of the methods well known in the art of pharmacy.

The compositions and methods of the invention may be administered to subjects by a variety of mucosal administration modes, including by oral, rectal, vaginal, intranasal, intrapulmonary, or transdermal delivery, or by topical delivery to the eyes, ears, skin or other mucosal surfaces. Compositions according to the present invention are often administered in an aqueous solution as a nasal or pulmonary spray and may be dispensed in spray form by a variety of methods known to those skilled in the art. Preferred systems for dispensing liquids as a nasal spray are disclosed in U.S. Pat. No. 4,511,069. Such formulations may be conveniently prepared by dissolving compositions according to the present invention in water to produce an aqueous solution, and rendering said solution sterile. The formulations may be presented in multi-dose containers, for example in the sealed dispensing system disclosed in U.S. Pat. No. 4,511,069. Other suitable nasal spray delivery systems have been described in Transdermal Systemic Medication, Y. W. Chen Ed., Elsevier Publishers, New York, 1985; and in U.S. Pat. No. 4,778,810. Additional aerosol delivery forms may include, e.g., compressed air-, jet-, ultrasonic-, and piezoelectric nebulizers, which deliver the biologically active agent dissolved or suspended in a pharmaceutical solvent, e.g., water, ethanol, or a mixture thereof.

Nasal and pulmonary spray solutions of the present invention typically comprise the drug or drug to be delivered, optionally formulated with a surface active agent, such as a nonionic surfactant (e.g., polysorbate-80), and one or more buffers, stabilizers, or tonicifiers. In some embodiments of the present invention, the nasal spray solution further comprises a propellant. The pH of the nasal spray solution is optionally between about pH 3.0 and 7.2, but when desired the pH is adjusted to optimize delivery of a charged macromolecular species (e.g., a therapeutic protein or peptide) in a substantially unionized state. The pharmaceutical solvents employed can also be a slightly acidic aqueous buffer (pH 3-6). Suitable buffers for use within these compositions are as described above or as otherwise known in the art. Other components may be added to enhance or maintain chemical stability, including preservatives, surfactants, dispersants, or gases. Suitable preservatives include, but are not limited to, phenol, methyl paraben, paraben, m-cresol, thiomersal, benzylalkonimum chloride, and the like. Suitable surfactants include, but are not limited to, oleic acid, sorbitan trioleate, polysorbates, lecithin, phosphotidyl cholines, and various long chain diglycerides and phospholipids. Suitable dispersants include, but are not limited to, ethylenediaminetetraacetic acid, and the like. Suitable gases include, but are not limited to, nitrogen, helium, chlorofluorocarbons (CFCs), hydrofluorocarbons (HFCs), carbon dioxide, air, and the like. Suitable stabilizers and tonicifying agents include sugars and other polyols, amino acids, and organic and inorganic salts.

The liquid transmucosal formulation can be administered as drops, e.g., installation, or as droplets (spray). The spray can be produced by pumps, nebulization, or by other methods as describe in the art. For pulmonary delivery, the liquid droplets for deep lung deposition exhibit a minimum particle size appropriate for deposition within the pulmonary passages is often about less than 10 μm mass median equivalent aerodynamic diameter (MMEAD), commonly about less than 5 μm MMEAD, commonly about less than about 2 μm MMEAD. For nasal delivery, the liquid droplet particle size is commonly about less than 1000 μm MMEAD, commonly less than 100 μm MMEAD.

Within alternate embodiments, mucosal formulations are administered as dry powder formulations comprising the biologically active agent in a dry, usually lyophilized, form of an appropriate particle size, or within an appropriate particle size range, for intranasal delivery. For pulmonary delivery, the powder particle for deep lung deposition exhibit a minimum particle size appropriate for deposition within the pulmonary passages is often about less than 10 μm mass median equivalent aerodynamic diameter (MMEAD), commonly about less than 5 μm MMEAD, commonly about less than about 2 μm MMEAD. For nasal delivery, the powder particle size is commonly about less than 1000 μm MMEAD, commonly less than 100 μm MMEAD. Intranasally respirable powders within these size ranges can be produced by a variety of conventional techniques, such as jet milling, spray drying, solvent precipitation, supercritical fluid condensation, and the like. These dry powders of appropriate MMEAD can be administered to a patient via a conventional dry powder inhaler (DPI) which relies on the patients breath, upon pulmonary or nasal inhalation, to disperse the power into an aerosolized amount. Alternatively, the dry powder may be administered via air assisted devices that use an external power source to disperse the powder into an aerosolized amount, e.g., a piston pump. The drug powder particles may be formulated in the dried state as particles agglomerated to large particles (>100 um MMEAD) comprising a suitable carrier, such as lactose, wherein the agglomerates of drug particles and carrier particles are disrupted upon dispensing the powder.

Dry powder devices typically require a powder mass in the range from about 1 mg to 20 mg to produce a single aerosolized dose (“puff”). If the required or desired dose of the biologically active agent is lower than this amount, the powdered active agent will typically be combined with a pharmaceutical dry bulking powder to provide the required total powder mass. Preferred dry bulking powders include sucrose, lactose, dextrose, mannitol, glycine, trehalose, human serum albumin (HSA), and starch. Other suitable dry bulking powders include cellobiose, dextrans, maltotriose, pectin, sodium citrate, sodium ascorbate, and the like.

To formulate compositions for mucosal delivery within the present invention, the biologically active agent can be combined with various pharmaceutically acceptable additives, as well as a base or carrier for dispersion of the active agent(s). Desired additives include, but are not limited to, pH control agents, such as arginine, sodium hydroxide, glycine, hydrochloric acid, citric acid, etc. In addition, local anesthetics (e.g., benzyl alcohol), isotonizing agents (e.g., sodium chloride, mannitol, sorbitol), adsorption inhibitors (e.g., Tween 80), solubility enhancing agents (e.g., cyclodextrins and derivatives thereof), stabilizers (e.g., serum albumin), and reducing agents (e.g., glutathione) can be included. When the composition for mucosal delivery is a liquid, the tonicity of the formulation, as measured with reference to the tonicity of 0.9% (w/v) physiological saline solution taken as unity, is typically adjusted to a value at which no substantial, irreversible tissue damage will be induced in the nasal mucosa at the site of administration. Generally, the tonicity of the solution is adjusted to a value of about 1/3 to 3, or 1/2 to 2, or 3/4 to 1.7.

The biologically active agent may be dispersed in a base or vehicle, which may comprise a hydrophilic compound having a capacity to disperse the active agent and any desired additives. The base may be selected from a wide range of suitable carriers, including but not limited to, copolymers of polycarboxylic acids or salts thereof, carboxylic anhydrides (e.g. maleic anhydride) with other monomers (e.g. methyl (meth)acrylate, acrylic acid, etc.), hydrophilic vinyl polymers such as polyvinyl acetate, polyvinyl alcohol, polyvinylpyrrolidone, cellulose derivatives such as hydroxymethylcellulose, hydroxypropylcellulose, etc., and natural polymers such as chitosan, collagen, sodium alginate, gelatin, hyaluronic acid, and nontoxic metal salts thereof. Often, a biodegradable polymer is selected as a base or carrier, for example, polylactic acid, poly(lactic acid-glycolic acid) copolymer, polyhydroxybutyric acid, poly(hydroxybutyric acid-glycolic acid) copolymer and mixtures thereof. Alternatively or additionally, synthetic fatty acid esters such as polyglycerin fatty acid esters, sucrose fatty acid esters, etc. can be employed as carriers. Hydrophilic polymers and other carriers can be used alone or in combination, and enhanced structural integrity can be imparted to the carrier by partial crystallization, ionic bonding, crosslinking and the like. The carrier can be provided in a variety of forms, including, fluid or viscous solutions, gels, pastes, powders, microspheres and films for direct application to the nasal mucosa. The use of a selected carrier in this context may result in promotion of absorption of the biologically active agent.

The biologically active agent can be combined with the base or carrier according to a variety of methods, and release of the active agent may be by diffusion, disintegration of the carrier, or associated formulation of water channels. In some circumstances, the active agent is dispersed in microcapsules (microspheres) or nanocapsules (nanospheres) prepared from a suitable polymer, e.g., isobutyl 2-cyanoacrylate (see, e.g., Michael, et al., J. Pharmacy Pharmacol. 43:1-5, 1991), and dispersed in a biocompatible dispersing medium applied to the nasal mucosa, which yields sustained delivery and biological activity over a protracted time.

To further enhance mucosal delivery of pharmaceutical agents within the invention, formulations comprising the active agent may also contain a hydrophilic low molecular weight compound as a base or excipient. Such hydrophilic low molecular weight compounds provide a passage medium through which a water-soluble active agent, such as a physiologically active peptide or protein, may diffuse through the base to the body surface where the active agent is absorbed. The hydrophilic low molecular weight compound optionally absorbs moisture from the mucosa or the administration atmosphere and dissolves the water-soluble active peptide. The molecular weight of the hydrophilic low molecular weight compound is generally not more than 10000 and preferably not more than 3000. Exemplary hydrophilic low molecular weight compound include polyol compounds, such as oligo-, di- and monosaccarides such as sucrose, mannitol, lactose, L-arabinose, D-erythrose, D-ribose, D-xylose, D-mannose, D-galactose, lactulose, cellobiose, gentibiose, glycerin and polyethylene glycol. Other examples of hydrophilic low molecular weight compounds useful as carriers within the invention include N-methylpyrrolidone, and alcohols (e.g. oligovinyl alcohol, ethanol, ethylene glycol, propylene glycol, etc.). These hydrophilic low molecular weight compounds can be used alone or in combination with one another or with other active or inactive components of the intranasal formulation.

The compositions of the invention may alternatively contain as pharmaceutically acceptable carriers substances as required to approximate physiological conditions, such as pH adjusting and buffering agents, tonicity adjusting agents, wetting agents and the like, for example, sodium acetate, sodium lactate, sodium chloride, potassium chloride, calcium chloride, sorbitan monolaurate, triethanolamine oleate, etc. For solid compositions, conventional nontoxic pharmaceutically acceptable carriers can be used which include, for example, pharmaceutical grades of mannitol, lactose, starch, magnesium stearate, sodium saccharin, talcum, cellulose, glucose, sucrose, magnesium carbonate, and the like.

In certain embodiments of the invention, the biologically active agent is administered in a time release formulation, for example in a composition which includes a slow release polymer. The active agent can be prepared with carriers that will protect against rapid release, for example a controlled release vehicle such as a polymer, microencapsulated delivery system or bioadhesive gel. Prolonged delivery of the active agent, in various compositions of the invention can be brought about by including in the composition agents that delay absorption, for example, aluminum monosterate hydrogels and gelatin.

The term “subject” as used herein means any mammalian patient to which the compositions of the invention may be administered.

### Kits

The instant invention also includes kits, packages and multicontainer units containing the above described pharmaceutical compositions, active ingredients, and/or means for administering the same for use in the prevention and treatment of diseases and other conditions in mammalian subjects. Briefly, these kits include a container or formulation that contains one or more biologically active agent formulated in a pharmaceutical preparation for mucosal delivery. The biologically active agent(s) is/are optionally contained in a bulk dispensing container or unit or multi-unit dosage form. Optional dispensing means may be provided, for example a pulmonary or intranasal spray applicator. Packaging materials optionally include a label or instruction indicating that the pharmaceutical agent packaged therewith can be used mucosally, e.g., intranasally, for treating or preventing a specific disease or condition.

### Polynucleotide Delivery Enhancing Polypeptides

Within additional embodiments of the invention, the polynucleotide delivery-enhancing polypeptide is selected or rationally designed to comprise an amphipathic amino acid sequence. For example, useful polynucleotide delivery-enhancing polypeptides may be selected which comprise a plurality of non-polar or hydrophobic amino acid residues that form a hydrophobic sequence domain or motif, linked to a plurality of charged amino acid residues that form a charged sequence domain or motif, yielding an amphipathic peptide.

In other embodiments, the polynucleotide delivery-enhancing polypeptide is selected to comprise a protein transduction domain or motif, and a fusogenic peptide domain or motif. A protein transduction domain is a peptide sequence that is able to insert into and preferably transit through the membrane of cells. A fusogenic peptide is a peptide that is able destabilize a lipid membrane, for example a plasma membrane or membrane surrounding an endosome, which may be enhanced at low pH. Exemplary fusogenic domains or motifs are found in a broad diversity of viral fusion proteins and in other proteins, for example fibroblast growth factor 4 (FGF4).

To rationally design polynucleotide delivery-enhancing polypeptides of the invention, a protein transduction domain is employed as a motif that will facilitate entry of the nucleic acid into a cell through the plasma membrane. In certain embodiments, the transported nucleic acid will be encapsulated in an endosome. The interior of endosomes has a low pH resulting in the fusogenic peptide motif destabilizing the membrane of the endosome. The destabilization and breakdown of the endosome membrane allows for the release of the siNA into the cytoplasm where the siNA can associate with a RISC complex and be directed to its target mRNA.

Examples of protein transduction domains for optional incorporation into polynucleotide delivery-enhancing polypeptides of the invention include:
1. TAT protein transduction domain (PTD) (SEQ ID NO: 1) KRRQRRR;
2. Penetratin PTD (SEQ ID NO: 2) RQIKWFQNRRMKWKK;
3. VP22 PTD (SEQ ID NO: 3) DAATATRGRSAASRPTERPRAPARSASRPRRPVD;
4. Kaposi FGF signal sequences (SEQ ID NO: 4) AAVALLPAVLLALLAP, and SEQ ID NO: 5) AAVLLPVLLPVLLAAP;
5. Human β3 integrin signal sequence (SEQ ID NO: 6) VTVLALGALAGVGVG;
6. gp41 fusion sequence (SEQ ID NO: 7) GALFLGWLGAAGSTMGA;
7. Caiman crocodylus Ig(v) light chain (SEQ ID NO: 8) MGLGLHLLVLAAALQGA;
8. hCT-derived peptide (SEQ ID NO: 9) LGTYTQDFNKFHTFPQTAIGVGAP;
9. Transportan (SEQ ID NO: 10) GWTLNSAGYLLKINLKALAALAKKIL;
10. Loligomer (SEQ ID NO: 11) TPPKKKRKVEDPKKKK;
11. Arginine peptide (SEQ ID NO: 12) RRRRRRR; and
12. Amphiphilic model peptide (SEQ ID NO: 13) KLALKLALKALKAALKLA.

Examples of viral fusion peptides fusogenic domains for optional incorporation into polynucleotide delivery-enhancing polypeptides of the invention include:
1. Influenza HA2 (SEQ ID NO: 14) GLFGAIAGFIENGWEG;
2. Sendai F1 (SEQ ID NO: 15) FFGAVIGTIALGVATA;
3. Respiratory Syncytial virus F1 (SEQ ID NO: 16) FLGFLLGVGSAIASGV;
4. HIV gp41 (SEQ ID NO: 17) GVFVLGFLGFLATAGS; and
5. Ebola GP2 (SEQ ID NO: 18) GAAIGLAWIPYFGPAA.

Within yet additional embodiments of the invention, polynucleotide delivery-enhancing polypeptides are provided that incorporate a DNA-binding domain or motif which facilitates polypeptide-siNA complex formation and/or enhances delivery of siNAs within the methods and compositions of the invention. Exemplary DNA binding domains in this context include various “zinc finger” domains as described for DNA-binding regulatory proteins and other proteins identified below (see, e.g., Simpson, et al., J. Biol. Chem. 278:28011-28018, 2003).

TABLE 1
Exemplary Zinc Finger Motifs of Different DNA-Binding Proteins

C₂H₂ Zinc finger motif

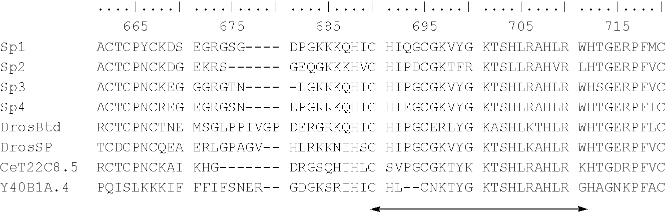

Prosite pattern
C-x(2,4)-C-x(12)-H-x(3)-H
*The table demonstrates a conservative zinc fingerer motif for double strand DNA binding which is characterized by the C-x(2,4)-C-x(12)-H-x(3)-H motif pattern, which itself can be used to select and design additional polynucleotide delivery-enhancing polypeptides according to the invention.
**The sequences shown in Table 1, for Sp1, Sp2, Sp3, Sp4, DrosBtd, DrosSp, CeT22C8.5, and Y4pB1A.4, are herein assigned SEQ ID NOs 19, 20, 21, 22, 23, 24, 25, and 26, respectively.

Alternative DNA binding domains useful for constructing polynucleotide delivery-enhancing polypeptides of the invention include, for example, portions of the HIV Tat protein sequence (see, Examples, below).

Within exemplary embodiments of the invention described herein below, polynucleotide delivery-enhancing polypeptides may be rationally designed and constructed by combining any of the foregoing structural elements, domains or motifs into a single polypeptide effective to mediate enhanced delivery of siNAs into target cells. For example, a protein transduction domain of the TAT polypeptide was fused to the N-terminal 20 amino acids of the influenza virus hemagglutinin protein, termed HA2, to yield one exemplary polynucleotide delivery-enhancing polypeptide herein. Various other polynucleotide delivery-enhancing polypeptide constructs are provided in the instant disclosure, evincing that the concepts of the invention are broadly applicable to create and use a diverse assemblage of effective polynucleotide delivery-enhancing polypeptides for enhancing siNA delivery.

Yet additional exemplary polynucleotide delivery-enhancing polypeptides within the invention may be selected from the following peptides:

WWETWKPFQCRICMRNFSTRQARRNHRRRHR (SEQ ID NO: 27); GKINLKALAALAKKIL (SEQ ID NO: 28), RVIRVWFQNKRCKDKK (SEQ ID NO: 29), GRKKRRQRRRPPQGRKKRRQRRRPPQGRKKRRQRRRPPQ (SEQ ID NO: 30), GEQIAQLIAGYIDIILKKKKSK (SEQ ID NO: 31), Poly Lys-Trp, 4:1, MW 20,000-50,000; and Poly Orn-Trp, 4:1, MW 20,000-50,000. Additional polynucleotide delivery-enhancing polypeptides that are useful within the compositions and methods herein comprise all or part of the mellitin protein sequence.

### EXAMPLES

The invention is illustrated by the examples below which do not limit the scope of the invention as described in the claims.

### Example 1

### Mucosal Delivery—Permeation Kinetics and Cytotoxicity

### Organotypic Model

The following methods are generally useful for evaluating mucosal delivery parameters, kinetics and side effects for a biologically active therapeutic agent and a mucosal delivery-enhancing effective amount of a permeabilizing peptide that reversibly enhances mucosal epithelial paracellular transport by modulating epithelial junctional structure and/or physiology in a mammalian subject.

The EpiAirway™ system was developed by MatTek Corp (Ashland, Mass.) as a model of the pseudostratified epithelium lining the respiratory tract. The epithelial cells are grown on porous membrane-bottomed cell culture inserts at an air-liquid interface, which results in differentiation of the cells to a highly polarized morphology. The apical surface is ciliated with a microvillous ultrastructure and the epithelium produces mucus (the presence of mucin has been confirmed by immunoblotting). The inserts have a diameter of 0.875 cm, providing a surface area of 0.6 cm². The cells are plated onto the inserts at the factory approximately three weeks before shipping. One “kit” consists of 24 units.

A. On arrival, the units are placed onto sterile supports in 6-well microplates. Each well receives 5 mL of proprietary culture medium. This DMEM-based medium is serum free but is supplemented with epidermal growth factor and other factors. The medium is always tested for endogenous levels of any cytokine or growth factor which is being considered for intranasal delivery, but has been free of all cytokines and factors studied to date except insulin. The 5 mL volume is just sufficient to provide contact to the bottoms of the units on their stands, but the apical surface of the epithelium is allowed to remain in direct contact with air. Sterile tweezers are used in this step and in all subsequent steps involving transfer of units to liquid-containing wells to ensure that no air is trapped between the bottoms of the units and the medium.

B. The units in their plates are maintained at 37° C. in an incubator in an atmosphere of 5% CO₂ in air for 24 hours. At the end of this time the medium is replaced with fresh medium and the units are returned to the incubator for another 24 hours.

### Experimental Protocol-Permeation Kinetics

A. A “kit” of 24 EpiAirway™ units can routinely be employed for evaluating five different formulations, each of which is applied to quadruplicate wells. Each well is employed for determination of permeation kinetics (4 time points), transepithelial electrical resistance (TER). An additional set of wells is employed as controls, which are sham treated during determination of permeation kinetics, but are otherwise handled identically to the test sample-containing units for determinations of transepithelial resistance and viability.

B. In all experiments, the mucosal delivery formulation to be studied is applied to the apical surface of each unit in a volume of 100 μL, which is sufficient to cover the entire apical surface. An appropriate volume of the test formulation at the concentration applied to the apical surface (no more than 100 μL is generally needed) is set aside for subsequent determination of concentration of the active material by ELISA or other designated assay.

C. The units are placed in 6 well plates without stands for the experiment: each well contains 0.9 mL of medium which is sufficient to contact the porous membrane bottom of the unit but does not generate any significant upward hydrostatic pressure on the unit.

D. In order to minimize potential sources of error and avoid any formation of concentration gradients, the units are transferred from one 0.9 mL-containing well to another at each time point in the study. These transfers are made at the following time points, based on a zero time at which the 100 μL volume of test material was applied to the apical surface: 15 minutes, 30 minutes, 60 minutes, and 120 minutes.

E. In between time points the units in their plates are kept in the 37° C. incubator. Plates containing 0.9 mL medium per well are also maintained in the incubator so that minimal change in temperature occurs during the brief periods when the plates are removed and the units are transferred from one well to another using sterile forceps.

F. At the completion of each time point, the medium is removed from the well from which each unit was transferred, and aliquotted into two tubes (one tube receives 700 μL and the other 200 μL) for determination of the concentration of permeated test material and, in the event that the test material is cytotoxic, for release of the cytosolic enzyme, lactate dehydrogenase, from the epithelium. These samples are kept in the refrigerator if the assays are to be conducted within 24 hours, or the samples are subaliquotted and kept frozen at −80° C. until thawed once for assays. Repeated freeze-thaw cycles are to be avoided.

G. In order to minimize errors, all tubes, plates, and wells are prelabeled before initiating an experiment.

H. At the end of the 120 minute time point, the units are transferred from the last of the 0.9 mL containing wells to 24-well microplates, containing 0.3 mL medium per well. This volume is again sufficient to contact the bottoms of the units, but not to exert upward hydrostatic pressure on the units. The units are returned to the incubator prior to measurement of transepithelial resistance.

Experimental Protocol—Transepithelial Electrical Resistance

A. Respiratory airway epithelial cells form tight junctions in vivo as well as in vitro, and thereby restrict the flow of solutes across the tissue. These junctions confer a transepithelial resistance of several hundred ohms×cm² in excised airway tissues. In the MatTek EpiAirway™ units, the transepithelial electrical resistance (TER) is reported by the manufacturer to be routinely around 1000 ohms×cm². Data determined herein indicates that the TER of control EpiAirway™ units which have been sham-exposed during the sequence of steps in the permeation study is somewhat lower (700-800 ohms×cm²), but, since permeation of small molecules is proportional to the inverse of the TER, this value is still sufficiently high to provide a substantial barrier to permeation. The porous membrane-bottomed units without cells, conversely, provide only minimal transmembrane resistance (approximately 5-20 ohms×cm²).

B. Accurate determinations of TER require that the electrodes of the ohmmeter be positioned over a significant surface area above and below the membrane, and that the distance of the electrodes from the membrane be reproducibly controlled. The method for TER determination recommended by MatTek and employed for all experiments herein employs an “EVOM”™ epithelial voltohmmeter and an “ENDOHM”™ tissue resistance measurement chamber from World Precision Instruments, Inc., Sarasota, Fla.

C. The chamber is initially filled with Dulbecco's phosphate buffered saline (PBS) for at least 20 minutes prior to TER determinations in order to equilibrate the electrodes.

D. Determinations of TER are made with 1.5 mL of PBS in the chamber and 350 μL of PBS in the membrane-bottomed unit being measured. The top electrode is adjusted to a position just above the membrane of a unit containing no cells (but containing 350 μL of PBS) and then fixed to ensure reproducible positioning. The resistance of a cell-free unit is typically 5-20 ohms×cm² (“background resistance”).

E. Once the chamber is prepared and the background resistance is recorded, units in a 24-well plate that had just been employed in permeation determinations are removed from the incubator and individually placed in the chamber for TER determinations.

F. Each unit is first transferred to a petri dish containing PBS to ensure that the membrane bottom is moistened. An aliquot of 350 μL PBS is added to the unit and then carefully aspirated into a labeled tube to rinse the apical surface. A second wash of 350 μL PBS is then applied to the unit and aspirated into the same collection tube.

G. The unit is gently blotted free of excess PBS on its exterior surface only before being placed into the chamber (containing a fresh 1.5 mL aliquot of PBS). An aliquot of 350 μL PBS is added to the unit before the top electrode is placed on the chamber and the TER is read on the EVOM meter.

H. After the TER of the unit is read in the ENDOHM chamber, the unit is removed, the PBS is aspirated and saved, and the unit is returned with an air interface on the apical surface to a 24-well plate containing 0.3 mL medium per well.

I. The units are read in the following sequence: all sham-treated controls, followed by all formulation-treated samples, followed by a second TER reading of each of the sham-treated controls. All TER values are reported as a function of the surface area of the tissue.

TER was calculated as:

TER=(R_{I}−R_{b})×A

Where R_{I} is resistance of the insert with a membrane, R_{b} is the resistance of the blank insert, and A is the area of the membrane (0.6 cm²). The effect of pharmaceutical formulations comprising intranasal delivery-enhancing agents, for example, permeabilizing peptides as measured by TER across the EpiAirway™ Cell Membrane (mucosal epithelial cell layer). Permeabilizing peptides are applied to the EpiAirway™ Cell Membrane at a concentration of 1.0 mM. A decrease in TER value relative to the control value (control=approximately 1000 ohms-cm²; normalized to 100.) indicates a decrease in cell membrane resistance and an increase in mucosal epithelial cell permeability.

### Experimental Protocol—LDH Assay

The amount of cell death was assayed by measuring the loss of lactate dehydrogenase (LDH) from the cells using a CytoTox 96 Cytoxicity Assay Kit (Promega Corp., Madison, Wis.). Fifty microliters of sample was loaded into a 96-well assay plates. Fresh, cell-free culture medium was used as a blank. 50 μl of substrate solution was added to each well and the plates incubated for 30 minutes at room temperature in the dark. Following incubation, 50 μl of stop solution was added to each well and the plates read on an optical density plate reader at 490 nm.

### Experimental Protocol—EIA Method

EIA kit (p/n S-1178(EIAH6101) was purchased from Peninsula Laboratories Inc. (Division of BACHEM, San Carlos, Calif., 800-922-1516). 17×120 mm polypropylene conical tubes (p/n 352097, Falcon, Franklin Lakes, N.J.) were used for all sample preparations. Eight standards were used for PTH quantitation. The rest of the assay procedure was the same as the kit inserts.

### Example 2

### Epithelial Permeation Enhancement by PN159

The examples herein below demonstrate that permeation enhancing peptides of the invention, exemplified by PN159, enhance mucosal permeation to peptide therapeutic drugs, including PTH and Peptide YY. This permeation enhancing activity of the peptides of the invention, as evinced for PN159, can be equivalent to, or greater than, epithelial permeation enhancement achieved through the use of one or multiple small molecule permeation enhancers.

Peptide YY₃₋₃₆ (PYY 3-36) is a 34 amino acid peptide which has been the subject of numerous clinical trials. Mucosal delivery of this biologically active peptide can be enhanced in formulations that include small molecule permeation enhancers. Accordingly, the instant studies assessed whether the permeation enhancing peptides of the invention, exemplified by PN159, could replace the role of small molecule permeation enhancers to facilitate mucosal delivery of peptide YY. These studies included evaluation of in vitro effects of PN159 to decrease Transepithelial Electrical Resistance (TEER) and increase permeation of marker substances, as well as related in vivo studies that proved consistent with the in vitro results.

In the current example, the combination of PN159 with PTH is described. PTH can be the full length peptide (1-84), or a fragment such as (1-34). The formulation can also be a combination of PTH, a permeabilizing peptide, and one or more other permeation enhancers. The formulation may also contain buffers, tonicifying agents, pH adjustment agents, and peptide/protein stabilizers such as amino acids, sugars or polyols, polymers, and salts.

The instant study was designed to evaluate the effect of PN159 itself or in combination with additional permeation enhancers on PTH permeation. The PN159 concentrations evaluated are 25, 50, and 100 μM. The additional permeation enhancers are 45 mg/ml M-β-CD, 1 mg/ml DDPC, and 1 mg/ml EDTA. Sorbitol was used as a tonicifier (146-190 mM) to adjust the osmolarity of formulations to 220 mOsm/kg. The formulation pH was fixed at 4.5. PTH was chosen as a model peptide in this example. 2 mg/ml PTH was combined with PN159 with or without additional permeation enhancers. The combination was tested using an in vitro epithelial tissue model to monitor PTH permeation, transepithelial electrical resistance (TER), and the cytotoxicity of the formulation by LDH assay.

### Transepithelial Electrical Resistance

The results of TER measurements from the present studies show more than 80% TER reduction caused by PN159. Higher TER reduction was observed with increasing PN159 concentration. Media applied to the apical side did not reduce TER whereas triton X treated group showed significant TER reduction as expected.

### Cytotoxicity

The data for LDH from the present studies shown no significant cytotoxicity was observed when cells were treated with 25-100 μM of PN159. Media applied to the apical side did not show cytotoxicity whereas the Triton X treated group showed significant cytotoxicity as expected.

### Permeation

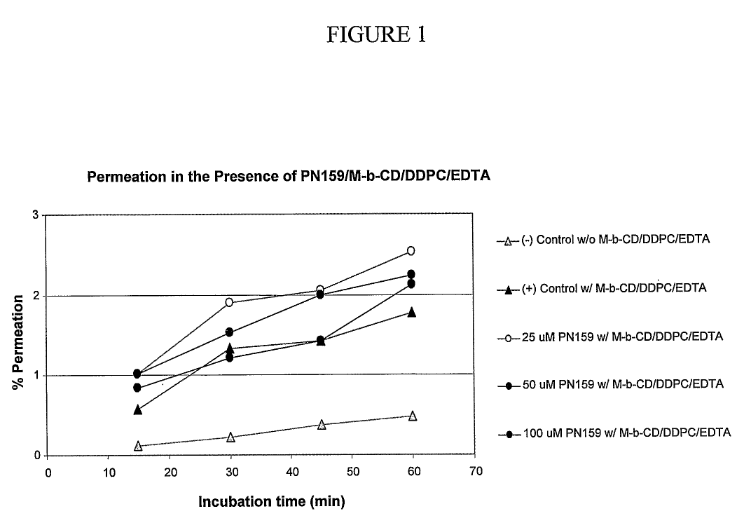

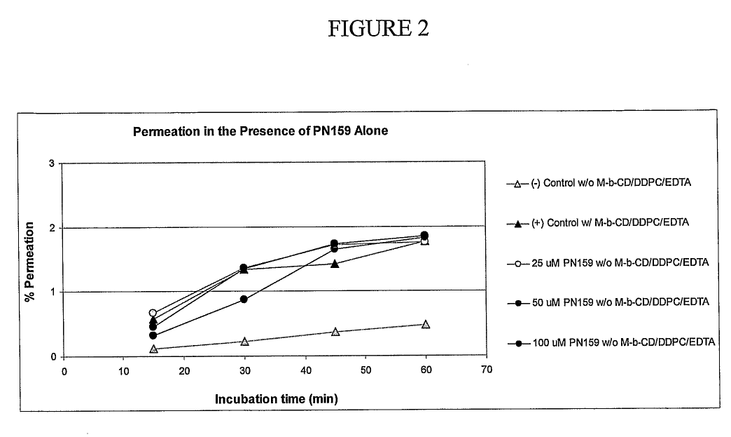

The PTH₁₋₃₄ permeation data for PN159 with and without additional enhancers are shown in FIGS. 1 and 2, respectively. Significant increase in PTH permeation was observed in the presence of PN159. No significant difference in % permeation was observed between 25, 50, and 100 μM PN159. Effect of PN159 on PTH permeation is comparable to 45/1/1 mg/ml M-β-CD/DDPC/EDTA. Additional increase in PTH permeation was observed with the combination of 45/1/1 mg/ml M-b-CD/DDPC/EDTA and PN159.

### Example 3

### In Vivo Permeation Enhancement by PN159 for a Peptide Hormone Therapeutic Agent Equals or Exceeds That of Small Molecule Permeation Enhancers

20 male New Zealand White rabbits age 3-6 months and weighing 2.1-3.0 kg were randomly assigned into one of 5 treatment groups with four animals per group. Test animals were dosed at 15 μl/kg and intranasally via pipette. Table 5 below indicates the composition of five different dose groups.

For dosing group 1 (see Table 2) a clinical formulation of PYY including small molecule permeation enhancers was used. The small molecule enhancers in these studies included methyl-βcyclodextrin, phosphatidylcholine didecanoyl (DDPC), and/or EDTA. Dosing group 2 received PYY dissolved in phosphate buffered saline (PBS). For dosing groups 3-5, various concentrations of PN 159 were added to dosing group 2, so that each of dosing groups 3-5 consisted of PYY, PN159, and PBS.

TABLE 2

| | | | Dose | Dose | PYY |
|---|---|---|---|---|---|
| | | | Conc | Vol | Dose |
| Group | Animals | Permeation enhancers | (mg/ml) | (ml/kg) | (μg/kg) |
| 1 | 4M | Small molecule | 13.67 | 0.015 | 205 |
| | | permeation enhancers | | | |
| 2 | 4M | None | 13.67 | 0.015 | 205 |
| 3 | 4M | 25 μM PN159 | 13.67 | 0.015 | 205 |
| 4 | 4M | 50 μM PN159 | 13.67 | 0.015 | 205 |
| 5 | 4M | 100 μM PN159 | 13.67 | 0.015 | 205 |

Serial blood samples (about 2 ml each) were collected by direct venipuncture from a marginal ear vein into blood collection tubes containing EDTA as an anticoagulant. Blood samples were collected at 0, 2.5, 5, 10, 15, 30, 45, 60, and 120 minutes post-dosing. After collection of the blood, the tubes were gently rocked several times for anti-coagulation, and then 50 μL aprotinin solution was added. The blood was centrifuged at approximately 1,600×g for 15 minutes at approximately 4° C., and plasma samples were dispensed into duplicate aliquots and stored frozen at approximately −70° C.

Averaging all four animals in a treatment group, the following plasma concentrations of PYY were measured (Table 3):

TABLE 3

| | Group 1 | Group 2 | | | |
|---|---|---|---|---|---|
| | Small molecule | No | Group 3 | Group 4 | Group 5 |
| Time, | permeation | permeation | 25 μM | 50 μM | 100 μM |
| mins | enhancers | enhancers | PN159 | PN159 | PN159 |
| 0 | 183.825 | 257.3 | 228.675 | 424.4 | 294.225 |
| 2.5 | 1280.7 | 242.8 | 526.375 | 749.975 | 1748.225 |
| 5 | 1449.425 | 273.675 | 1430.15 | 1293.4 | 3088.2 |
| 10 | 8251.8 | 372.05 | 6521.7 | 12517.2 | 14486.6 |
| 15 | 13731.2 | 398.225 | 12563.075 | 34455.3 | 20882.725 |
| 30 | 19537.55 | 476.475 | 15222.6 | 35294.375 | 25470.475 |
| 45 | 13036.075 | 340.7 | 9081.125 | 21582.225 | 16499.55 |
| 60 | 7080.875 | 283.825 | 4843.15 | 9461.925 | 10676.625 |
| 120 | 1671.9 | 192.575 | 1224.2 | 2337.775 | 1891.275 |

The pharmacokinetic data calculated from the above data is shown below in Table 4:

TABLE 4

| Variable | Group | Mean | SD | SE |
|---|---|---|---|---|
| Cmax (pg/mL) | 1 | 19832.18 | 17737.21 | 8868.605 |
| Tmax (min) | 1 | 32.5 | 20.6155 | 10.3078 |
| AUClast | 1 | 991732.1 | 930296.3 | 465148.1 |
| (min * pg/mL) | | | | |
| AUCINF | 1 | 1357132 | 928368.5 | 535993.8 |
| (min * pg/mL) | | | | |
| t½ (min) | 1 | 23.69 | 1.713 | 0.989 |
| Cmax (pg/mL) | 2 | 516.725 | 196.492 | 98.246 |
| Tmax (min) | 2 | 26.25 | 14.3614 | 7.1807 |
| AUClast | 2 | 36475.72 | 9926.104 | 4963.052 |
| (min * pg/mL) | | | | |
| AUCINF | 2 | 60847.41 | 17688.31 | 8844.156 |
| (min * pg/mL) | | | | |
| t½ (min) | 2 | 84.5919 | 26.8859 | 13.4429 |
| Cmax (pg/mL) | 3 | 15533.95 | 13225.88 | 6612.941 |
| Tmax (min) | 3 | 22.5 | 8.6603 | 4.3301 |
| AUClast | 3 | 748104.1 | 661213.8 | 330606.9 |
| (min * pg/mL) | | | | |
| AUCINF | 3 | 796354.7 | 721017.8 | 360508.9 |
| (min * pg/mL) | | | | |
| t½ (min) | 3 | 24.8467 | 4.3108 | 2.1554 |
| Cmax (pg/mL) | 4 | 40995.53 | 32112.71 | 16056.35 |
| Tmax (min) | 4 | 26.25 | 7.5 | 3.75 |
| AUClast | 4 | 1692499 | 1339896 | 669947.8 |
| (min * pg/mL) | | | | |
| AUCINF | 4 | 1787348 | 1395185 | 697592.4 |
| (min * pg/mL) | | | | |
| t½ (min) | 4 | 25.5355 | 8.6139 | 4.3069 |
| Cmax (pg/mL) | 5 | 27974.4 | 17584.31 | 8792.154 |
| Tmax (min) | 5 | 33.75 | 18.8746 | 9.4373 |
| AUClast | 5 | 1384241 | 817758.8 | 408879.4 |
| (min * pg/mL) | | | | |
| AUCINF | 5 | 1518949 | 1030623 | 595030.3 |
| (min * pg/mL) | | | | |
| t½ (min) | 5 | 20.4628 | 6.5069 | 3.7568 |

Compared with the Group 2 (no enhancer) formulation, the following relative enhancement ratios were determined (Table 5):

TABLE 5

| | | Relative | Relative |
|---|---|---|---|
| Group | Formulation | Cmax | AUC last |
| 1 | Small molecule permeation enhancers | 38x | 27x |
| 3 | PN159, 25 μm | 30x | 21x |
| 4 | PN159, 50 μm | 79x | 46x |
| 5 | PN159, 100 μm | 54x | 38x |

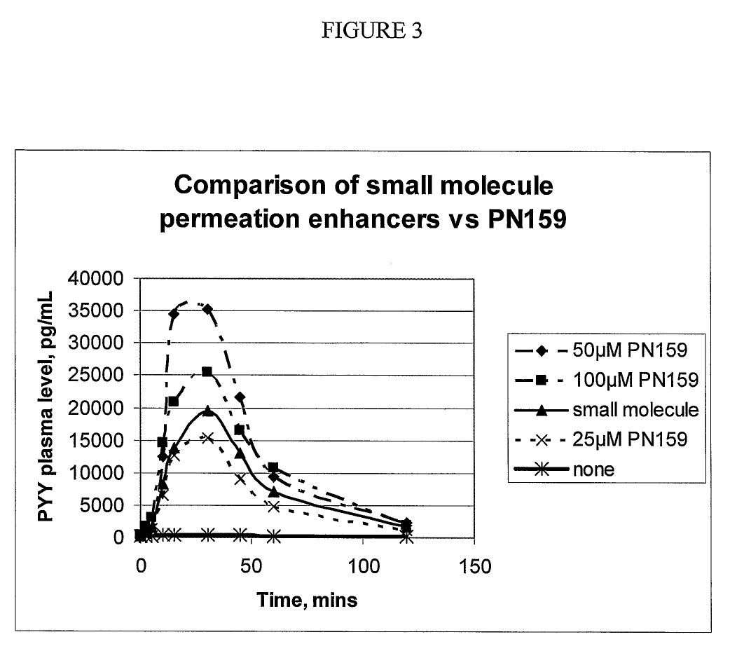

The foregoing data are graphically depicted in FIG. 3, and demonstrate that permeabilizing peptides of the invention, as exemplified by PN159, are able to enhance in vivo intranasal permeation of a human hormone peptide therapeutic to an equal or greater degree compared to small molecule permeation enhancers. The greatest effect of the peptide is seen at a 50 μM concentration. The 100 μM concentration resulted in somewhat less permeation, although both resulted in higher permeation than the small molecule permeation enhancers.

### Example 4

### Permeation Enhancement by PN159 for an Oligopeptide Therapeutic Agent

The present example demonstrates efficacy of an exemplary peptide of the invention, PN159 to enhance epithelial permeation for a cyclic pentapeptide, melanocortin-4 receptor agonist (MC-4RA) a model oligopeptide agonist for a mammalian cellular receptor. In this example, a combination of one or more of the permeabilizing peptides with MC-4RA is described. Useful formulations in this context can include a combination of an oligopeptide therapeutic, a permeabilizing peptide, and one or more other permeation enhancers. The formulation may also contain buffers, tonicifying agents, pH adjustment agents, and peptide/protein stabilizers such as amino acids, sugars or polyols, polymers, and salts.

The effect of PN159 on permeation of MC-4RA was evaluated in this study. MC-4RA was a methanesulphonate salt with a molecular weight of approximately 1,100 Da, which modulates activity of the MC-4 receptor. The PN159 concentrations evaluated are 5, 25, 50, and 100 μM. 45 mg/ml M-β-CD was used as a solubilizer for all formulations to achieve 10 mg/ml peptide concentration. The effect of PN159 was assessed either by itself or in combination with EDTA (1, 2.5, 5, or 10 mg/ml). The formulation pH was fixed at 4 and the osmolarity was at 220 mOsm/kg.

### HPLC Method

The concentrations of MC-4RA in the basolateral media was analyzed by the RP-HPLC using a C18 RP chromatography with a flow rate of 1 mL/minute and a column temperature of 25° C.
Solvent A: 0.1% TFA in water; Solvent B: 0.1% TFA in ACN
Injection Volume: 50 μL
Detection: 220 nm
RUN TIME: 15 MIN

MC-4RA was combined with 5, 25, 50, and 100 μM PN159, pH 4 and osmolarity ˜220 mOsm/kg. The combination was tested using an in vitro epithelial tissue model to monitor PTH permeation, transepithelial electrical resistance (TER), and the cytotoxicity of the formulation by MTT and LDH assays.

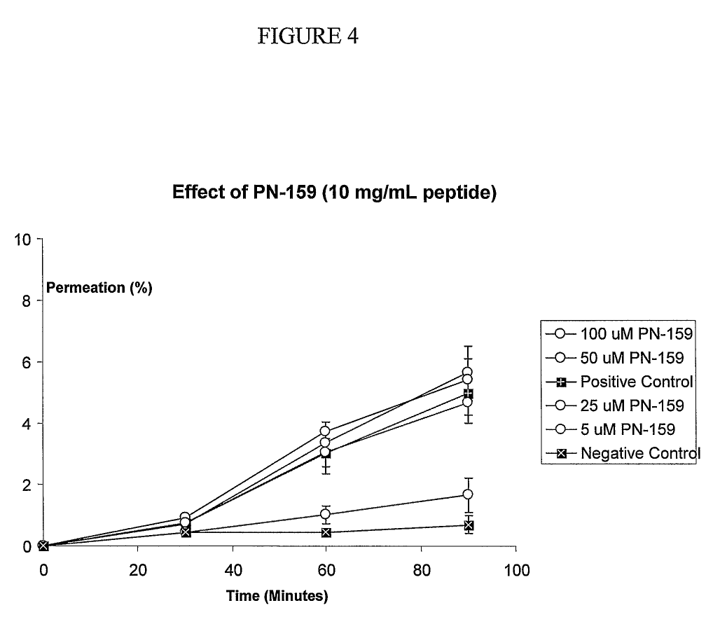

The results of studies of the permeation of MC-4RA are shown in FIG. 4. These studies evince that PN159, in addition to enhancing mucosal permeation for peptide hormone therapeutics, also significantly enhance epithelial permeation for oligopeptide therapeutic agents.

### Example 5

### Permeation Enhancement by PN159 for a Small Molecule Drug

The present example demonstrates efficacy of an exemplary peptide of the invention, PN159 to enhance epithelial permeation for a small molecule drug, exemplified by the acetylcholinesterase (ACE) inhibitor galantamine. In this example, a combination of one or more of the permeabilizing peptides with a small molecule drug is described. Useful formulations in this context can include a combination of a small molecule drug, a permeabilizing peptide, and one or more other permeation enhancers. The formulation may also contain buffers, tonicifying agents, pH adjustment agents, stabilizers and/or preservatives.

The present invention combines galantamine with PN159 to enhance permeation of galantamine across the nasal mucosa. This increase in drug permeation is unexpected because galantamine is a small molecule that can permeate the nasal epithelial membrane independently. The significant enhancement of galantamine permeation across epithelia mediated by addition of excipients which enhance the permeation of peptides is therefore surprising, on the basis that such excipients would not ordinarily be expected to significantly increase permeation of galantamine across the epithelial tissue layer. The invention therefore will facilitate nasal delivery of galantamine and other small molecule drugs by increasing their bioavailability.

In the present studies, 40 mg/ml galantamine in the lactate salt form was combined with 25, 50, and 100 μM PN159 in solution, pH 5.0 and osmolarity ˜270 mOsm. The combination was tested using an in vitro epithelial tissue model to monitor galantamine permeation, transepithelial electrical resistance (TER), and the cytotoxicity of the formulation by LDH and MTT assays as described above. Permeation measurements for galantamine were conducted by standard HPLC analysis, as follows.

### HPLC Analysis

Galantamine concentration in the formulation and in the basolateral media (permeation samples) was determined using an isocratic LC (Waters Alliance) method with UV detection.
Column: Waters Symmetry Shield, C18, 5 um, 25×0.46 cm
Mobile phase: 5% ACN in 50 mM ammonium formate, pH 3.0
Flow rate: 1 ml/min
Column temperature: 30° C.
Calibration curve: 0-400 μg/ml Galantamine HBr
Detection: UV at 285 nm

Based on the foregoing studies, PN159 improves transmucosal delivery of small molecules. Galantamine was chosen as a model low molecular weight drug, and the results for this molecule are considered predictive of permeabilizing peptide activity for other small molecule drugs. To evaluate permeabilizing activity in this context, 40 mg/ml galantamine in the lactate salt form was combined with 25, 50, and 100 μM PN159 in solution, pH 5.0 and osmolarity ˜270 mOsm. The combination was tested using an in vitro epithelial tissue model to monitor galantamine permeation, transepithelial electrical resistance (TER), and the cytotoxicity of the formulation by LDH and MTT assays.

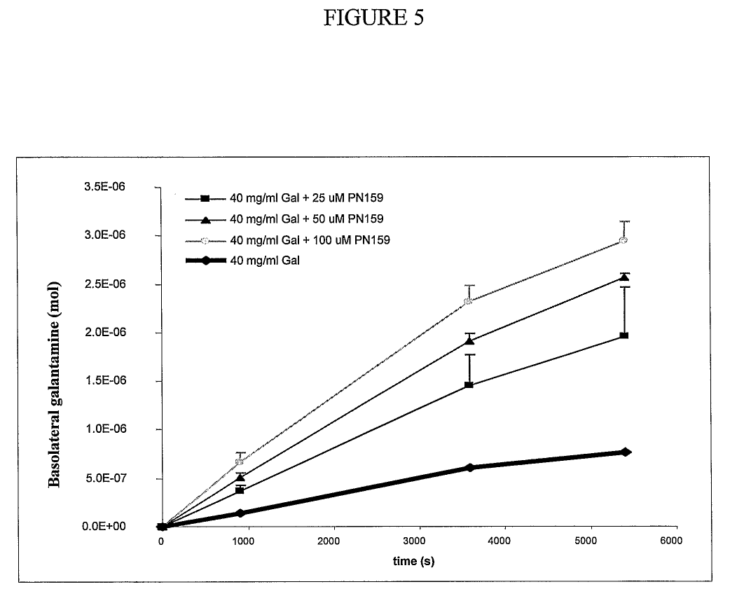

In the in vitro tissue model, the addition of PN159 resulted in a dramatic increase in drug permeation across the cell barrier. Specifically, there was a 2.5-3.5 fold increase in the Pₐₚₚ of 40 mg/ml galantamine. (FIG. 5)

PN159 reduced TER in the presence of galantamine just as described in Example II.

Cell viability remained high (>80%) in the presence of galantamine lactate and PN159 at all concentrations tested. Conversely, cytotoxicity was low in the presence of PN159 and galantamine lactate, as measured by LDH. Both of these assays suggest that PN159 is not toxic to the epithelial membrane.

Summarizing the foregoing results, PN159 has been demonstrated herein to surprisingly increase epithelial permeation of galantamine as a model low molecular weight drug. The addition of PN159 to galantamine in solution significantly enhances galantamine permeation across epithelial monolayers. Evidence shows that PN159 temporarily reduces TER across the epithelial membrane without damaging the cells in the membrane, as measured by high cell viability and low cytotoxicity. PN159 therefore is an exemplary peptide for enhancing bioavailability of galantamine and other small molecule druges in vivo, via the same mechanism that is demonstrated herein using in vitro models. It is further expected that PN159 will enhance permeation of galantamine at higher concentrations as well.

### Chemical Stability

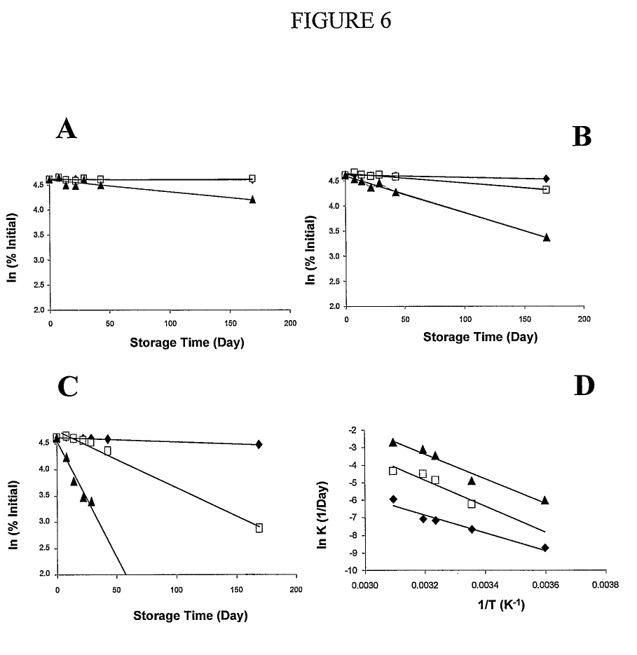

The chemical stability of the PN159 was determined under therapeutically relevant storage conditions. A stability indicating HPLC method was employed. Solutions (50 mM) were stored at various pH (4.0, 7.3, and 9.0) and temperature (5° C., 25° C., 35° C., 40° C., and 50° C.) conditions. Samples at pH 4 contained 10 mM citrate buffer. Samples at pH 7.3 and 9.0 contained 10 mM phosphate buffer. Representative storage stability data (including the Arrhenius plot) are depicted in FIG. 6. As can be seen, the PN159 was most chemically stable at low temperature and pH. For example, at 5° C. and pH 4.0 or pH7.3, there was essentially 100% recovery of PN159 for six month storage. When the storage temperature was increased to 25° C., there was a 7% and 26% loss of native PN159 for samples at pH 4 or pH 7, respectively, after six months. At pH 9 and/or at elevated temperature, e.g., 40 to 50° C., rapid deterioration of the PN159 ensued. The pH range of 4.0 to 7.3 and the temperature range of refrigerated to ambient are most relevant for intranasal formulations. Therefore, these data support that the PN159 can maintain chemical integrity under storage conditions relevant to IN formulations. There was a marked increase in rate of drug permeated vs. time. These data were used to calculate the permeability constant (Pₐₚₚ), presented in Table 6.

TABLE 6
Pₐₚₚ Measured Using the In Vitro Tissue Model

| Drug Formulation | [PN159] (μM) | Papp (cm/s) | Relative Pₐₚₚ |
|---|---|---|---|
| Galantamine | 0 | 2.1 × 10⁻⁶ | 1.0 |
| 40 mg/mL, pH 5.0 | 25 | 5.1 × 10⁻⁶ | 2.4 |
| | 50 | 6.2 × 10⁻⁶ | 3.0 |
| | 100 | 7.2 × 10⁻⁶ | 3.4 |
| Calcitonin | 0 | 9.7 × 10⁻⁸ | 1.0 |
| 1 mg/mL, pH 3.5 | 25 | 2.2 × 10⁻⁶ | 23. |
| | 50 | 3.3 × 10⁻⁶ | 34. |
| | 100 | 4.6 × 10⁻⁶ | 47. |
| PTH₁₋₃₄ | 0 | 1.1 × 10⁻⁷ | 1.0 |
| 1 mg/mL, pH 4.5 | 25 | 3.4 × 10⁻⁷ | 3.0 |
| | 50 | 4.9 × 10⁻⁷ | 4.5 |
| | 100 | 4.3 × 10⁻⁷ | 3.9 |
| PYY₃₋₃₆ | 0^{a} | 1.3 × 10⁻⁷ | 1.0 |
| 1 mg/mL, pH 7.0 | 25 | 1.6 × 10⁻⁶ | 12. |
| | 100 | 2.2 × 10⁻⁶ | 17. |

^{a}pH was 5.0

In the absence of PN159, the Pₐₚₚ for galantamine was about 2.1×10⁻⁶ cm/s. In the presence of 25, 50 and 100 mM PN159, Pₐₚₚ was 5.1×10⁻⁶, 6.2×10⁻⁶, and 7.2×10⁻⁶ cm/s, respectively. Thus, the PN159 afforded a 2.4- to 3.4-fold increase in Pₐₚₚ of this model low-molecular-weight drug.

Having established the utility of the PN159 for transmucosal formulations of low-molecular-weight compounds, it was important to discern whether these observations could be extrapolated to larger molecules, e.g., therapeutic peptides and proteins. For this purpose, in vitro tissue studies were performed on salmon calcitonin as a model therapeutic peptide in the absence and presence of 25, 50, and 100 mM PN159. In the absence of PN159, the Pₐₚₚ for calcitonin was about 1×10⁻⁷ cm/s, about an order of magnitude lower than that for galantamine, presumably due to the difference in molecular weight. The data reveal a dramatic increased in calcitonin permeation in the presence of the PN159, up to a 23- to 47-fold increase in Pₐₚₚ compared to the case of the calcitonin alone (Table 6).

In order to explore the generality of these findings, two additional peptides, namely human parathyroid hormone 1-34 (PTH₁₋₃₄) and human peptide YY 3-36 (PYY₃₋₃₆) were examined in the in vitro model in the absence and presence of PN159 (Pₐₚₚ data presented in Table 6). In the absence of PN159, the Pₐₚₚ of these two peptides was consistent to that for calcitonin. In the case of PTH₁₋₃₄, the presence of PN159 afforded about 3-5 fold increase in Pₐₚₚ. When PYY₃₋₃₆ was formulated in the presence of PN159, the Papp was increased about 12- to 17-fold. These data confirm the generality of our finding that the PN159 has utility for enhancing transmucosal drug delivery.

### Example 6

### D-amino acid versions of PN159

The D-amino acid substituted PN159 peptides listed in Table 7 were synthesized and purified, and were tested for their ability to enhance TER and permeability, using the methods described in the Examples above.

TABLE 7
D-Amino Acid Substitutions

| | | | TER(x)/ | Perm(x)/ |
|---|---|---|---|---|
| | | | TER(159) +/− | Perm(159) +/− |
| Peptide | Sequence | Description | SEM | SEM |
| PN159 | NH2- | model | 1.00 +/− 0.14 | 1.00 +/− 0.13 |
| | KLALKLALKALKAALKLA- | amphipathic | | |
| | amide | peptide | | |
| | (SEQ ID NO: 34) | | | |
| PN393 | NH2-klalklalkalkaalkla-amide | All D- | 1.06 +/− 0.00 | 1.02 +/− 0.16 |
| | (SEQ ID NO: 35) | substituted | | |
| PN407 | NH2-LKlLKkLlkKLLkLL- | Leucine and | 1.08 +/− 0.01 | 1.20 +/− 0.05 |
| | amide | Lysine rich | | |
| | (SEQ ID NO: 36) | with D-subs | | |
| PN434 | NH2-KLaLKlALkAlkAALkLA- | D-substituted | 0.12 +/− 0.01 | 0.02 +/− 0.00 |
| | amide | | | |
| | (SEQ ID NO: 37) | | | |
| PN408 | NH2-alklaaklaklalklalk-amide | PN159 retro- | 1.05 +/− 0.01 | 1.16 +/− 0.07 |
| | (SEQ ID NO: 38) | inverso | | |

PN407 shows minor but statistically significant improvement on permeability. Both All D and retro inverso forms of PN159 show decreased TER recovery suggesting a longer TER reduction effect that might be useful for in vivo delivery. Random D substitution (PN434) can cause null activities both on TER reduction and permeability enhancement.

### Example 7

### PN159 Length Changes

PN159 peptides having length changes listed in Table 8 were synthesized and purified, and were tested for their ability to enhance TER and permeability, using the methods described in the Examples above.

TABLE 8
Different Sizes

| | | | TER(x)/ | Perm (x)/ |
|---|---|---|---|---|
| | | | TER(159) +/− | Perm (159) |
| Peptide | Sequence | Description | SEM* | +/− SEM* |
| PN159 | NH2-KLALKLALKALKAALKLA-amide | model peptide | 1.00 +/− 0.14 | 1.00 +/− 0.13 |
| PN417 | NH2-KLALKLALKALKAA-amide | Shortened 14aa | 0.19 +/ 0.01 | 0.04 +/− 0.01 |
| | (SEQ ID NO: 39) | | | |
| PN418 | NH2-KLALKLALKALKAALK-amide | Shortened 16 aa | 1.05 +/− 0.05 | 0.64 +/− 0.08 |
| | (SEQ ID NO: 40) | | | |
| PN419 | NEH2-KLALKLALKALKAALKLALK-aimde | Lengthened 20 aa | 1.23 +/− 0.01 | 0.74 +/− 0.13 |
| | (SEQ ID NO: 41) | | | |
| PN420 | NH2-KLALKLALKALKAALKLALKLA-amide | Lengthened 22 aa | 0.77 +/− 0.05 | 0.24 +/− 0.05 |
| | (SEQ ID NO: 42) | | | |
| PN421 | NH2-KLALKLALKALKAALKLALKLALK-amide | Lengthened 24 aa | 0.74 +/− 0.11 | 0.17 +/− 0.06 |
| | (SEQ ID NO: 43) | | | |
| PN422 | NH2-KLALKALKALKAALKLkLKLNLKAL-amide | Lengthened 26 aa | 0.47 +/− 0.07 | 0.07 +/− 0.01 |
| | (SEQ ID NO: 44) | | | |

*mean values from multiple repeats

The results show that lengths of PN159 is important for its TER reduction and enhanced permeability activity. Lengthen PN159 to 20 aa increased TER reduction effect but reduced permeability effect. TER recovery is slower. Shorten PN159 to 16 aa show no effect on TER reduction but reduced permeability effect. Shorten PN159 to 14 aa drastically reduced permeability, suggesting the length of PN159 is crucial of permeability. Contrary to the permeability effect, the effect of the PN159 length on TER reduction is more gradual.

### Example 8

### Trytophan and Arginine Substitutions in PN159

PN159 peptides having amino acid substitutions listed in Table 9 were synthesized and purified, and were tested for their ability to enhance TER and permeability, using the methods described in the Examples above.

TABLE 9
Amino Acid Substitutions

| | | | | |
|---|---|---|---|---|
| | | | Relative TER | Relative |
| Peptide | Sequence | Name | Decrease | Permeability |

| | | | | |
|---|---|---|---|---|
| PN159 | NH2-KLALKLALKALKAALKLA-amide | model peptide | 1.0 | 1.0 |
| PN394 | NH2-RLALRLALRALRAALRLK-amide | Argenine | 0.7 | 0.1 |
| | (SEQ ID NO: 45) | | | |
| PN395 | NH2-RLAWRLALRALRAALRLA-amide | Argenine and Single | 0.8 | 0.2 |
| | (SEQ ID NO: 46) | Tryptophan | | |
| PN0425 | NH2-KLAWKLALKALKAALKLA-amide | Single Tryptophan | 1.0 | 1.2 |
| | (SEQ ID NO: 47 | | | |
| PN0427 | NH2-KLAWKLALKALKAAWKLA-amide | Two Tryptophan | 1.0 | 1.0 |
| | (SEQ ID NO: 48 | | | |
| PN0428 | NH2-KLAWKLAWKALKAAWKLA-amide | Three Tryptophan | 0.7 | 1.0 |
| | (SEQ ID NO: 49 | | | |
| PN406 | NH2-LKLLKKLLKKLLKLL-amide | Leucine and Lysine rich | 0.9 | 0.6 |
| | (SEQ ID NO: 50 | | | |
| PN407 | NH2-LK1LKkL1kKLLkLL-amide | Leucine and Lysine rich | 1.1 | 1.2 |
| | | with D-subs | | |
| PN443 | NH2-LKTLATALTKLAKTLTTL-amide | Threonine | 0.3 | 0.1 |
| | (SEQ ID NO: 51) | | | |
| PN448 | NH2-KLALKLALKNLKAALKLA-amide | Asparagine | 0.4 | 0.0 |
| | (SEQ ID NO: 52 | | | |

The results show that an arginine guanidinium headgroup is more effective than lysine and histidine. Tryptophan is preferential amino acid at the water-membrane interface1. PN407 shows minor but statistically significant improvement on permeability. Arginine replacement of Lysine drastically reduce the permeability but has less impact on TER reduction, suggesting the importance of Lysine is permeability. Single replacement of Alanine on aa10 with Asparagine abolish permeability, suggesting the important of alpha helicy for PN159 activities.

### Example 9

### Hydrophobicity Changes in PN159

PN159 peptides having amino acid substitutions listed in Table 10 were synthesized and purified, and were tested for their ability to enhance TER and permeability, using the methods described in the Examples above.

TABLE 10
Hydrophobic Faces

| | | | TER(x)/TER(159) | Perm(x)/Perm(159) |
|---|---|---|---|---|
| Peptide | Sequence | Description | +/− SEM* | +/− SEM* |
| PN159 | NH2-KLALKLALKALKAALKLA-amide | model | 1.00 +/− 0.14 | 1.00 +/− 0.13 |
| | peptide | | | |
| PN424 | NH2-KALKLKAALALLAKLKLA-amide | non- | 0.59 +/− 0.07 | 0.20 +/− 0.04 |
| | (SEQ ID NO: 53) | amphipatbic | | |
| PN441 | NH2-KLAAALLKKAKKLAAALL-amide | 200° | 0.54 +/− 0.04 | 0.35 +/− 0.04 |
| | (SEQ ID NO: 54) | hydrophobic | | |
| | | face | | |
| PN442 | NH2-KALAALLKKAAKLLAALK-amide | 180° face | 0.93 +/− 0.03 | 0.81 +/− 0.03 |
| | (SEQ ID NO: 55) | | | |
| PN444 | NH2-KALAALLKKLAKLLAALK-amide | 180° face | 0.82 +/− 0.05 | 0.41 +/− 0.08 |
| | (SEQ ID NO: 56) | | | |

* mean values from multiple repeats

PN159 has 280 degrees of hydrophobic faces. The results show that reduction of the hydrophobic faces can cause reduction of PN159 activities. Amphipathicity of PN159 is also important for its activities.

### In Vitro Methods and Protocols.

Each TAR was assayed for transepithelial electrical resistance (TER), TER recovery, cytotoxicity (LDH), and sample permeation (EIA). The cell culture conditions and protocols for each assay are explained below in detail.

### Example 10

### In Vitro Methods and Protocols

Tight junction modulating peptides or TJMPs are peptides capable of compromising the integrity of tight junctions with the effect of creating openings between epithelial cells and thus reducing the barrier function of an epithelia. The state of tight junction integrity can be assayed in vitro by measuring the level of electrical resistance and degree sample permeation across a human nasal epithelial tissue model system. A reduction in electrical resistance and enhanced permeation suggests that the tight junctions have been compromised and openings have been created between the epithelial cells. In effect, peptides that induce a measured reduction in electrical resistance across a tissue membrane, referred to as (TER) reduction, and promote enhanced permeation of a small molecule through a tissue membrane are classified as TJMPs. In addition, the level of cell toxicity for TJMPs is also assessed to determine whether these peptides could function as tight junction modulating peptides in drug delivery across a mucosal surface, for example intranasal (IN) drug delivery.

The assays used to screen the exemplary peptides of the present invention (refer to Table 23 of Example 25) are described in the present example. These assays include transepithelial electrical resistance (TER), cytotoxicity (LDH), and sample permeation. Also described are the reagents used and the cell culture conditions.

Table 11 illustrates the sample reagents used in the subsequent Examples.

TABLE 11
Sample Reagents

| Reagent | Grade | Manufacturer | City, State | Lot # | MW |
|---|---|---|---|---|---|
| 1X DPBS++ | TC | Gibco/Invitrogen ™ | Carlsbad, | 1213061 | |
| | | | CA | | |
| Sterile, Nulcease- | | Ambion ™ | Austin, TX | 065P053618A | |
| Free Water | | | | | |
| Fluorescent | | Molecular | Carlsbad, | 111105 | 3000 |
| Dextran | | Probes/Invitrogen ™ | CA | | |
| Air-100 | TC | MatTek ™ | Ashland, | 11110565 | |
| Medium ™ | | | MA | | |
| Air-196 inserts ™ | | MatTek ™ | Ashland, | 7118 | |
| | | | MA | | |
| CytoTox 96 | | Promega ™ | Madison, WI | 210634 | |
| Assay ™ | | | | | |

TC = tissue culture

### Cell Cultures

The EpiAirway™ system was developed by MatTek Corp. (Ashland, Mass.) as a model of the pseudostratified epithelium lining the respiratory tract. The epithelial cells are grown on porous membrane-bottomed cell culture inserts at an air-liquid interface, which results in differentiation of the cells to a highly polarized morphology. The apical surface is ciliated with a microvillous ultrastructure and the epithelium produces mucus (the presence of mucin has been confirmed by immunoblotting). The cells are plated onto the inserts at the factory approximately three weeks before shipping.

EpiAirway™ culture membranes were received the day before the experiments started. They are shipped in phenol red-free and hydrocortisone-free Dulbecco's Modified Eagle's Medium (DMEM). The cells are ciliated and psudostratefied, grown to confluency on Millipore Multiscreen Caco-2 96-well assay system comprised of a polycarbonate filter system. Upon receipt, the insert system will be stored unopened at 4° C. and/or cultured in 250 μl basal media per well (phenol red-free and hydrocortisone-free Dulbecco's Modified Eagle's Medium (DMEM)) at 37° C./5% CO2 for 24 hours before use.

This model system was used to evaluate the efficacy of TJMPs to modulate TEER, effect cytotoxicity and enhance permeation of an epithelial cell monolayer.

The cell line MatTek Corp. (Ashland, Mass.) will be the source of normal, human-derived tracheal/bronchial epithelial cells (EpiAirway™ Tissue Model). The cells are provided as inserts grown to confluency on Millipore Milicell-CM filters comprised of transparent hydrophilic Teflon (PTFE). Upon receipt, the membranes are cultured in 1 ml basal media (phenol red-free and hydrocortisone-free Dulbecco's Modified Eagle's Medium (DMEM) at 37° C./5% CO2 for 24-48 hours before use. Inserts are feed for each day of recovery.

Madin-Darbey canine kidney cells (MDCK), human intestinal epithelial cells (Caco-2), and human bronchial epithelial cells (16HBE114o-) cells were seeded in Multi-Screen Caco-2 96-well inserts from Millipore. These cells were grown as a monolayer and under similar conditions as the EpiAirway epithelial cells.

### Peptide Synthesis

Peptide syntheses were performed on a Rainin Symphony synthesizer on a 50 umol scale using NovaBiochem TGR resin. Deprotections were performed by two treatments of 20% piperidine in DMF for 10 minutes. After deprotection the resin was washed once with 10 mL DMF containing 5% HOBt (30 s) and 4 times with 10 mL DMF (30 s). Couplings were performed by delivering 5-fold excess Fmoc amino acid in DMF to the reaction vessel followed by delivery of an equal volume of activator solution containing 6.25-fold excess N-methylmorpholine and 5-fold excess of HCTU. A coupling time of 40 mins was used throughout the synthesis. After the first coupling reaction the resin was washed twice with 10 mL of DMF (30 s) prior to initiating the second coupling step. For pegylated peptides, upon completion of the peptide synthesis the N-terminal Fmoc group was removed and 2 equivalents of O-(N-Fmoc-2-aminoethyl)-O′-(2-carboxyethyl)-undecaethyleneglycol in DMF were added manually to the reaction vessels. While in manual mode, 2 equivalents of activator solution were delivered to the reaction vessel and the coupling was allowed to proceed overnight. Generally, coupling efficiencies of greater than 97% was achieved and any unreacted peptide was capped by acetic anhydride.

Cleavage was performed on the individual reaction vessels by delivery of 10 mL of TFA containing 2.5% TIS, 2.5% water followed by gentle nitrogen agitation for 3 h. The cleavage solution was collected automatically into conical tubes, pooled and the volume was reduced by evaporation under reduced pressure. The resulting solution was triturated with an excess of cold ether, filtered and washed extensively with cold ether. After drying, the crude peptide was taken up in Millipore water and lyophilized to dryness.

FITC (fluorescein-5-isotbiocyanate)-Dextran Permeation Assay

A FITC labeled dextran with a molecular weight 3000 (FD3) was used to assess the efficacy of individual TJMP on epithelial cell monolayer permeation. The tissue insert plates were transferred to a 96-well receiver plate containing 200 μl of DPBS++ as basal media. The apical surface of each tissue culture insert was incubated with a 20 μl sample of a single test formulation (refer to Table 24 of Example 25 for details of test formulations) for one hour at 37° C. in the dark on a shaker (˜100 rpm). Following the 1-hour incubation period, underlying basal media samples were taken from each tissue culture insert and temporarily stored in the dark at room temperature until FD3 levels were quantified by fluorescence spectroscopy. For FD3 measurements, a 150 μl of basal media sample was transferred to a black, clear bottom 96-well plate. Fluorescence emission at 528/20 following excitation at 485/20 were measured using a FL×800 fluorescence plate reader from Biotek Instruments.

Permeation was calculated as:

$$\% Permeation = \frac{Cb \times Vb}{Ca \times Va} \times 100$$

$$Apparent Permeability ( Papp ) , cm \text{/} sec = \frac{Vb}{SA \times Ca} \frac{Cb}{dt}$$

Formula terms for permeation defined:

Cb: Basolateral concentration

Ca: Apical Concentration

Vb: Basolateral Volume

Va: Apical Volume

SA: Filter Surface Area

dt: Elapsed Time

Each tissue insert will be placed in an individual well containing 1 ml of MatTek basal media. On the apical surface of the inserts, 25 μl of test formulation will be applied according to study design, and the samples will be placed on a shaker (˜100 rpm) for 1.5 h at 37° C. FITC-labeled dextran solution is added to inserts apically and a fluorescence measurement is made from the basolateral media after the incubation period. The concentration of FITC-dextran is expressed as a percent of the starting material applied to the cells. A FITC labeled dextran with a molecular weight 4000 (MW4000) was used to assess cargo size limitations on individual TJMP permeation. Of note, various size FITC-labeled dextrans are available to perform size limitation studies.

### Transepithelial Electrical Resistance (TER) and TER Recovery

TER measurements will be accomplished using the Endohm-12 Tissue Resistance Measurement Chamber connected to the EVOM Epithelial Voltohmmeter (World Precision Instruments, Sarasota, Fla.) with the electrode leads. The electrodes and a tissue culture blank insert will be equilibrated for at least 20 minutes in MatTek medium with the power off prior to checking calibration. The background resistance will be measured with 1.5 ml Media in the Endohm tissue chamber and 300 μl Media in the blank insert. The top electrode will be as adjusted so that it is close to, but not making contact with, the top surface of the insert membrane. Background resistance of the blank insert should be about 5-20 ohms. For each TER determination, 300 μl of MatTek medium will be added to the insert followed by placement in the Endohm chamber All TER values are reported as a function of the surface area of the tissue.

TER was calculated as:

TER=(R_{I}−R_{b})×A

Where R_{I} is resistance of the insert with a membrane, R_{b} is the resistance of the blank insert, and A is the area of the membrane (0.6 cm²). A decrease in TER value relative to the control value (control=approximately 1000 ohms-cm²; normalized to 100.) indicates a decrease in cell membrane resistance and an increase in mucosal epithelial cell permeability.

For TER recovery, TER's were measured at 1, 3, 5, and 21 hours post treatment. Percent TER was calculated as:

% TER=(TER T_{post treatment}/TER T₀)/(TER T_{post treatment}/TER T₀ for media control).

In some embodiments, TER measurements were taken using the REMS Autosampler (World Precision Instruments, Sarasota, Fla.) with the electrode leads. The electrodes and a tissue culture blank insert will be equilibrated for at least 20 minutes in MatTek Air-100™ medium with the power off prior to checking calibration. The background resistance of the insert system has been established by multiple measurements of a blank insert plate and the same value was used for each test on the platform. Time zero TER (TER0) was measured before incubation of the inserts with the test formulation. The top electrode will be as adjusted so that it is close to, but not making contact with, the top surface of the insert membrane. Background resistance of the blank insert should be about 5-20 ohms. For each TER determination, 100 μl of MatTek Air-100™ medium was added to the insert and 250 μl in the basal well followed by placement in the Endohm chamber. All TER values are reported as a function of the surface area of the tissue. Resistance was expressed as both Ohms*cm2 and percent original TER value.

TER values were calculated as:

$$Nominal Resistance , Ohm * {cm}^{2} = ( TERt - blank ) * 0.12$$

$$Relative TER , \% = \frac{TERt - blank}{TER 0 - blank} \times 100$$

Formula terms for TER calculation defined:

TER0: TER measurement at time zero.

TERt: TER measurement taken at time t after test formulation incubation

blank: Background resistance measurement

A decrease in TER value relative to the control value indicates a decrease in cell membrane resistance and an increase in mucosal epithelial cell permeability.

### Cytotoxicity (LDH Assay)

The amount of cell death will be assayed by measuring the loss of lactate dehydrogenase (LDH) from the cells using a CytoTox 96 Cytotoxicity Assay Kit (Promega Corp., Madison, Wis.). Fifty microliters of sample will be loaded into a 96-well assay plates. Fresh, cell-free culture medium will be used as a blank. Fifty microliters of substrate solution will be added to each well and the plates incubated for 30 minutes at room temperature in the dark. Following incubation, 50 μl of stop solution will be added to each well and the plates read on an optical density plate reader at 490 nm. The measurement of LDH release into the basolateral media indicates relative cytotoxicity of the samples. One hundred percent lysis of control inserts with 0.3% Octylphenolpoly(ethyleneglycolether)×(TritonX-100) allows LDH values to be expressed as percentage of total lysis.

Alternatively, cytoxicity can be measured using a WST-1 assay. The WST-1 assay measure cell viability based on mitochondrial metabolic activity. The apical side of the cell monolayer was incubated with the WST-1 reagent (Roche) for 4 hours at 37° C. following peptide treatment, washing, and TER measurement at 10 minutes post treatment. Apical cell supernatants were measured at OD 450 nm using a microplate reader. % Values=sample_{OD 450}/media control_{OD 450}.

In some embodiments, The amount of cell death was assayed by measuring the release of lactate dehydrogenase (LDH) from the cells into the apical medium using a CytoTox 96 Cytotoxicity Assay Kit (Promega Corp., Madison, Wis.). One percent Octylphenolpoly (ethyleneglycolether)×(Triton X-100™) diluted in phosphate buffered saline (PBS) causes 100% lysis in cultured cells and served herein as a positive control for the LDH assay. Following the one hour incubation period with a test formulation (refer to Table 24 of Example 25 for details of test formulations), the total liquid volume of each insert was brought to a final volume of 200 μl with culture medium. The apical medium was then mixed by pipetting four times with a multichannel pipette set to a 100 μl volume. After mixing, a 100 μl sample from the apical side of each insert was transferred to a new 96-well plate. The apical media samples were sealed with a plate sealer and stored at room temperature for same day analysis or stored overnight at 4° C. for analysis the next day. To measure LDH levels, 5 μl of the 100 μl apical media sample was diluted in 45 μl DPBS in a new 96-well plate. Fresh, cell-free culture medium will be used as a blank. Fifty microliters of substrate solution was added to each well and incubated for 30 minutes at room temperature away from direct light. Following the 30 minute incubation, 50 μl of stop solution was added to each well. Optical density (OD) was measured at 490 nm with a uQuant absorbance plate reader from Biotek Instruments. The measurement of LDH release into the apical media indicates relative cytotoxicity of the samples. Percent cytotoxicity for each test formulation was calculated by subtracting the measured absorbance of the PBS control (basal level of LDH release) from the measured absorbance of the individual test formulation and then dividing that value by the measured absorbance for the 1% Triton X-100™ positive control, multiplied by 100.

The formula used to calculate percent cytotoxicity is as follows:

$$Relative Cytotoxicity , \% = \frac{ODx - ODpbs}{ODtriton} \times 100$$

### Osmolality

Samples were measured by Model 20200 from Advanced Instruments Inc. (Norwood, Mass.).

### Example 11

### Peptides that Modulate Epithelial Tight Junctions and Enhance Epithelial Cell Layer Permeation In Vitro

Table 12 shows the amino acid sequence of 11 peptides that modulate tight junction proteins and enhance epithelial cell layer permeation in vitro as measured by TER assay and permeation kinetics. For the purposes of these Examples, PN27 was chosen to represent both PN27 and PN28 because of their similar activities.

TABLE 12

| | |
|---|---|
| Peptide | Amino Acid Sequence |

| | |
|---|---|
| PN159 | NH2-KLALKLALKALKAALKLA-amide |
| PN161 | NH2-GWTLNSAGYLLGKINLKALAALAKKIL-amide |
| | (SEQ ID NO: 63) |
| PN202 | NH2-LLETLLKPFQCRICMRNFSTRQARRNHRRRHRR- |
| | amide |
| | (SEQ ID NO: 64) |
| PN27 | NH2-AAVALLPAVLLALLAPRKKRRQRRRPPQ-amide |
| | (SEQ ID NO: 65) |
| PN28 | NH2-RKKRRQRRRPPQCAAVALLPAVLLALLAP-amide |
| | (SEQ ID NO: 66) |
| PN58 | NH2-RQIKIWFQNRRMKWKK-amide |
| | (SEQ ID NO: 67) |
| PN73 | NH2-KGSKKAVTKAQKKDGKKRKRSRKESYSVYVYKVLKQ- |
| | amide |
| | (SEQ ID NO: 68) |
| PN228 | NH2-KLWSAWPSLWSSLWKP-amide |
| | (SEQ ID NO: 69) |
| PN250 | NH2-RRRQRRKRGGDIMGEWGNEIFGAIAGFLG-amide |
| | (SEQ ID NO: 70) |
| PN283 | Maleimide-GLGSLLKKAGKLKLKQPKSKRKV-amide |
| | (SEQ ID NO: 71) |
| PN183 | NH2-KETWWETWWTEWSQPGRKKRRQRRRRPPQ-amide |
| | (SEQ ID NO: 72) |

### Example 12

### Tight Junction Modulating Peptides Reduce TER

The present example evaluated the efficacy of various peptides to modulate tight junction proteins in an epithelial cell monolayer in vitro as assayed by TER reduction. A summary of the TER data obtained from experiments performed in EpiAirway epithelial cells for each TJMP is presented in Table 13. The highlighted boxes in the table represent the highest TER reduction observed for that TJMP within the concentration range tested.

TABLE 13

| Peptide | 1000 μM | 500 μM | 250 μM | 125 μM | 100 μM | 50 μM | 25 μM | 10 μM | 2.5 μM | 1 μM |
|---|---|---|---|---|---|---|---|---|---|---|
| PN159 | | | | | | | 94% | 89% | 79% | 54% |
| PN161 | | | | | 84% | | 73% | 43% | 11% | |
| PN202 | | 95% | | | 95% | 57% | | 3% | | |
| PN27 | | 94% | 91% | 81% | | | | | | |
| PN283 | 92% | 86% | | | 39% | | | | | |
| PN250 | | 84% | 79% | | 58% | | 17% | 3% | | |
| PN228 | | 82% | | | 9% | | | | | |
| PN73 | | 83% | | | 38% | 8% | | | | |
| PN58 | 88% | 64% | −6% | | | | | | | |
| PN183 | 55% | 41% | | | 25% | | | | | |

PN159, PN202, PN27, and PN283 reduced TER in excess of 90% while PN161, PN250, PN228, PN73, and PN58 reduced TER by 82% to 88%. PN28 is not shown, but it functionally equivalent to PN27. Finally PN183 had a TER reduction of 55%. These data indicate that all tested TJMPs are capable of compromising epithelial cell tight junctions in vitro.

In addition, a TER recovery analysis was done to determine the rate at which the EpitAirway epithelial cell layer recovers after treatment with the TJMPs. Surprisingly, the results indicate that PN250, PN202, and PN161 have the fastest recovery time of all TJMPs tested. These data indicate that the effect of TJMPs on the epithelial cell layer is transient in nature.

### Example 13

### In Vitro Permeation Kinetics of Tight Junction Modulating Peptides

In this example, the efficacy of TJMPs to mediate EpiAirway epithelial cell permeation was addressed. Table 14 below shows a summary of the permeation kinetics for each TJMP shown in percent permeation. The highlighted boxes in the table represent the greatest degree of permeation observed for that TJMP within the concentration range tested.

TABLE 14

| Peptide | 1000 μM | 500 μM | 250 μM | 125 μM | 100 μM | 50 μM | 25 μM | 10 μM | 2.5 μM | 1 μM |
|---|---|---|---|---|---|---|---|---|---|---|
| PN159 | | | | | | | 12.5% | 6.5% | 1.9% | 0.6% |
| PN161 | | | | | 7.1% | | 2.9% | 1.6% | 0.3% | |
| PN202 | | 5.9% | | | 2.8% | 1.5% | | 0.2% | | |
| PN27 | | 8.4% | 7.3% | 7.7% | | | | | | |
| PN283 | 5.2% | 3.9% | | | 0.7% | | | | | |
| PN250 | | 4.2% | 3.3% | | 1.7% | | 0.5% | 0.3% | | |
| PN228 | | 1.7% | | | 0.2% | | | | | |
| PN73 | | 0.8% | | | 0.2% | 0.1% | | | | |
| PN58 | 6.3% | 4.5% | 0.9% | | 0.3% | 0.3% | | | | |
| PN183 | 0.6% | 0.5% | | | 0.2% | | | | | |

These data indicate that all TJMPs tested are able to enhance in vitro permeation of an epithelial cell monolayer. In general, the degree of permeability correlates with the peptides ability to reduce TER.

### Example 14

### Tight Junction Modulating Peptides do not Cause Significant Cytotoxicity

The present example evaluated the cytotoxic effect on epithelial cells after exposure to TJMPs. An LDH assay was performed after a 15 minute and 60 minute treatment with each peptide. In all instances, after a 15 minute treatment almost no LDH release was observed. After a 60 minute treatment, cytotoxicity levels varied among the tested peptides but were within acceptable levels indicating all peptides tested do not cause significant cell injury.

### Example 15

### TER Reduction by Tight Junction Modulating Peptides is Consistent Among All Epithelial Cell Types Tested

To determine whether the TER results observed in the EpiAirway epithelial cell culture system were representative of other epithelial cell types, MDCK, Caco-2, and 16HBE14o-cells were treated with the TJMPs and assayed for TER. In all instances, TER results observed with these cell types were consistent with TER results observed with EpiAirway epithelial cells indicating that these TJMPs have the capacity to reduce TER among all epithelial cell types.

### Example 16

### Tight Junction Modulating Peptides Ranked Based on Performance

Nine TJMPs were ranked and categorized into 4 different performance tiers according to their level of permeability, TER values, rate of TER recovery, and cytoxicity as shown in Table 15. PN183 and PN28 were not included in Table 15. The table below summarizes each TJMPs' optimal concentration (i.e., greatest degree of TER reduction associated with the highest level of permeability and showed no significant cytotoxicity) and the corresponding percent permeation after a 15 minute treatment of the EpiAirway epithelial cells with the peptide and after a 60 minute treatment of the EpitAirway epithelial cells with the peptide. In addition, LDH values (cytotoxicity) for a 15 minute and 60 minute treatment are shown for each peptide. The TER recovery is also shown. The TER recovery rate directly correlates with the slope value (i.e., greater slope value correlates with faster TER recovery).

TABLE 15

| | | Optimal | | | | | TER Recovery | |
|---|---|---|---|---|---|---|---|---|
| | Peptide | Concentration | % Perm 15 | % Perm 60 | LDH15 | LDH60 | Slope | |
| Tier I | PN161 | 100 uM | 2.82% | 7.42% | 0.0017 | 0.01 | 74.81 | high permeability |
| | PN159 | 25 uM | 2.72% | 8.01% | 0.007 | 0.002 | 65.44 | low toxicity, swift recovery |
| Tier II | PN27 | 250 uM | 3.12% | 7.31% | 0.0056 | 0.035 | 62.19 | high permeability |
| | PN228 | 500 uM | 2.67% | 6.99% | 0.0063 | 0.046 | 49.59 | moderate toxicity |
| Tier III | PN250 | 500 uM | 1.99% | 5.19% | 0.0016 | 0.031 | 88.94 | lower permeability |
| | PN202 | 100 uM | 1.39% | 4.44% | 0.0011 | 0.02 | 78.52 | swift recovery, low tox. |
| Tier IV | PN58 | 500 uM | 0.60% | 5.66% | 0.0007 | 0.02 | 61.24 | low permeability |
| | PN73 | 500 uM | 0.23% | 2.20% | 0.0006 | 0.005 | 65.29 | slowest recovery |
| | PN283 | 1000 uM | 1.06% | 4.99% | 0.0007 | 0.032 | 62.32 | low toxicity |

### Example 17

### Tight Junction Modulating Peptides Enhance Permeation of FITC-Dextran MW4000 across an Epithelial Cell Monolayer

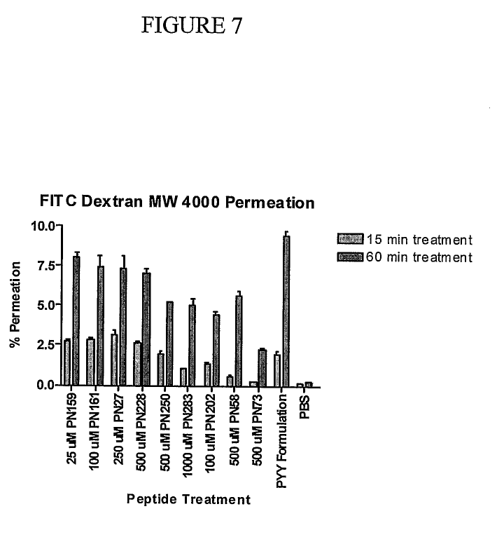

In this example, a study was done to determine the permeation kinetics of FITC-dextran MW4000 in the presence of each TJMP. This experiment assessed whether permeation was dependent upon the incubation time of the peptide with the epithelial cell monolayer and whether permeation is cargo size dependent. Cell permeation was assayed after a 15 minute treatment of the cells and also after a 60 minute treatment of the cells with a TJMP and the FITC-dextran MW4000 (FIG. 7). The PYY formulation was used as the positive control and phosphate buffered saline (PBS) was used as the negative control. The peptides were tested at a concentration that demonstrated the greatest degree of TER reduction associated with the highest level of permeability and showed no significant cytotoxicity.

The 60 minutes treatment showed a significantly higher degree of permeation than the 15 minute treatment for the same TJMP. Surprisingly PN161, PN127, and PN228 showed a level of permeation equivalent to PN159 (approximately 7.5%). The TJMPs PN250, PN283, PN202, PN58 achieved approximately 5% permeation after 60 minutes of incubation with the cells, which is just short of the permeation achieved by PN161, PN127, PN228 and PN159. These date indicate that all TJMPs tested are capable of enhancing the permeation of FITC-dextran MW4000 and this enhancement is dependent upon how long the peptide is in contact with the epithelial cell layer.

The forgoing experiments demonstrate that the tested TJMPs are able to enhance in vitro permeation of an epithelial cell monolayer.

### Example 18

### Enhanced Permeation In Vitro by a Tight Junction Modulating Peptide Correlates Strongly with Enhanced Permeation Observed In Vivo

A linear regression analysis was performed to determine whether the TJMP permeation kinetics observed in the in vitro EpiAirway epithelial cell model system correlated with the in vivo pharmacokinetic data observed for that same TJMP. To determine if in vitro permeation data functions as a good indicator for success in vivo, the area under the curve-last value (AUC-last) derived from in vivo pharmacokinetic studies done with PYY and TJMPs was plotted against in vitro epithelial cell monolayer permeation studies done with PYY and TJMPs. In vitro permeation was expressed as a percentage and AUC-last as Min*pg/ml. In vitro and in vivo studies for 10 different TJMPs were graphed and a linear regression performed. An R² value of 0.82 (82% correlation) was derived indicating a strong correlation exist for AUC values derived in vivo and percent permeability observed in vitro. Surprisingly, when inter-assay variability is excluded, an R² value of 0.996 (essentially 100%) was derived indicating a direct correlation exist between in vitro permeability and in vivo success. Thus, in vitro permeation can be used to predict in vivo success.

### Example 19

### In Vivo Permeation Enhancement by a TJMP for a Peptide Hormone Therapeutic Agent Equals or Exceeds That of Small Molecule Permeation Enhancers

Twenty male New Zealand White rabbits age 3-6 months and weighing 2.1-3.0 kg were randomly assigned into one of 5 treatment groups with four animals per group. Test animals were dosed at 15 μl/kg and intranasally via pipette. Table 19 below indicates the composition of five different dose groups.

For dosing group 1 (see Table 16) a clinical formulation of PYY including small molecule permeation enhancers was used. The small molecule enhancers in these studies included methyl-β-cyclodextrin, phosphatidylcholine didecanoyl (DDPC), and/or EDTA. Dosing group 2 received PYY dissolved in phosphate buffered saline (PBS). For dosing groups 3-5, various concentrations of PN159 were added to dosing group 2, so that each of dosing groups 3 to 5 consisted of PYY, PN159, and PBS.

TABLE 16

| | | | Dose | Dose | PYY |
|---|---|---|---|---|---|
| | | | Conc | Vol | Dose |
| Group | Animals | Permeation enhancers | (mg/ml) | (ml/kg) | (μg/kg) |
| 1 | 4M | Small molecule | 13.67 | 0.015 | 205 |
| | | permeation enhancers | | | |
| 2 | 4M | None | 13.67 | 0.015 | 205 |
| 3 | 4M | 25 μM PN159 | 13.67 | 0.015 | 205 |
| 4 | 4M | 50 μM PN159 | 13.67 | 0.015 | 205 |
| 5 | 4M | 100 μM PN159 | 13.67 | 0.015 | 205 |

Serial blood samples (about 2 ml each) were collected by direct venipuncture from a marginal ear vein into blood collection tubes containing EDTA as an anticoagulant. Blood samples were collected at 0, 2.5, 5, 10, 15, 30, 45, 60, and 120 minutes post-dosing. After collection of the blood, the tubes were gently rocked several times for anti-coagulation, and then 50 μl aprotinin solution was added. The blood was centrifuged at approximately 1,600×g for 15 minutes at approximately 4° C., and plasma samples were dispensed into duplicate aliquots and stored frozen at approximately −70° C.

Averaging all four animals in a treatment group, the following plasma concentrations of PYY were measured (Table 17):

TABLE 17

| | Group 1 | | | | |
|---|---|---|---|---|---|
| | Small | Group 2 | | | |
| | molecule | No | Group 3 | Group 4 | Group 5 |
| Time, | permeation | permeation | 25 μM | 50 μM | 100 μM |
| mins | enhancers | enhancers | PN159 | PN159 | PN159 |
| 0 | 183.825 | 257.3 | 228.675 | 424.4 | 294.225 |
| 2.5 | 1280.7 | 242.8 | 526.375 | 749.975 | 1748.225 |
| 5 | 1449.425 | 273.675 | 1430.15 | 1293.4 | 3088.2 |
| 10 | 8251.8 | 372.05 | 6521.7 | 12517.2 | 14486.6 |
| 15 | 13731.2 | 398.225 | 12563.075 | 34455.3 | 20882.725 |
| 30 | 19537.55 | 476.475 | 15222.6 | 35294.375 | 25470.475 |
| 45 | 13036.075 | 340.7 | 9081.125 | 21582.225 | 16499.55 |
| 60 | 7080.875 | 283.825 | 4843.15 | 9461.925 | 10676.625 |
| 120 | 1671.9 | 192.575 | 1224.2 | 2337.775 | 1891.275 |

The pharmacokinetic data calculated from the above data is shown below in Table 18:

TABLE 18

| Variable | Group | Mean | SD | SE |
|---|---|---|---|---|
| Cmax (pg/mL) | 1 | 19832.18 | 17737.21 | 8868.605 |
| Tmax (min) | 1 | 32.5 | 20.6155 | 10.3078 |
| AUClast | 1 | 991732.1 | 930296.3 | 465148.1 |
| (min * pg/mL) | | | | |
| AUCINF | 1 | 1357132 | 928368.5 | 535993.8 |
| (min * pg/mL) | | | | |
| t½ (min) | 1 | 23.69 | 1.713 | 0.989 |
| Cmax (pg/mL) | 2 | 516.725 | 196.492 | 98.246 |
| Tmax (min) | 2 | 26.25 | 14.3614 | 7.1807 |
| AUClast | 2 | 36475.72 | 9926.104 | 4963.052 |
| (min * pg/mL) | | | | |
| AUCINF | 2 | 60847.41 | 17688.31 | 8844.156 |
| (min * pg/mL) | | | | |
| t½ (min) | 2 | 84.5919 | 26.8859 | 13.4429 |
| Cmax (pg/mL) | 3 | 15533.95 | 13225.88 | 6612.941 |
| Tmax (min) | 3 | 22.5 | 8.6603 | 4.3301 |
| AUClast | 3 | 748104.1 | 661213.8 | 330606.9 |
| (min * pg/mL) | | | | |
| AUCINF | 3 | 796354.7 | 721017.8 | 360508.9 |
| (min * pg/mL) | | | | |
| t½ (min) | 3 | 24.8467 | 4.3108 | 2.1554 |
| Cmax (pg/mL) | 4 | 40995.53 | 32112.71 | 16056.35 |
| Tmax (min) | 4 | 26.25 | 7.5 | 3.75 |
| AUClast | 4 | 1692499 | 1339896 | 669947.8 |
| (min * pg/mL) | | | | |
| AUCINF | 4 | 1787348 | 1395185 | 697592.4 |
| (min * pg/mL) | | | | |
| t½ (min) | 4 | 25.5355 | 8.6139 | 4.3069 |
| Cmax (pg/mL) | 5 | 27974.4 | 17584.31 | 8792.154 |
| Tmax (min) | 5 | 33.75 | 18.8746 | 9.4373 |
| AUClast | 5 | 1384241 | 817758.8 | 408879.4 |
| (min * pg/mL) | | | | |
| AUCINF | 5 | 1518949 | 1030623 | 595030.3 |
| (min * pg/mL) | | | | |
| t½ (min) | 5 | 20.4628 | 6.5069 | 3.7568 |

Compared with the Group 2 (no enhancer) formulation, the following relative enhancement ratios were determined (Table 19):

TABLE 19

| | | Relative | Relative AUC |
|---|---|---|---|
| Group | Formulation | Cmax | last |
| 1 | Small molecule permeation enhancers | 38x | 27x |
| 3 | PN159, 25 μm | 30x | 21x |
| 4 | PN159, 50 μm | 79x | 46x |
| 5 | PN159, 100 μm | 54x | 38x |

The foregoing data demonstrate that TJMP enhances in vivo intranasal permeation of a human hormone peptide therapeutic to an equal or greater degree compared to small molecule permeation enhancers. The greatest effect of the peptide is seen at a 50 μM concentration. The 100 μM concentration resulted in somewhat less permeation, although both resulted in higher permeation than the small molecule permeation enhancers.

### Example 20

### Permeation Enhancement by TJMP for an Oligopeptide Therapeutic Agent

The present example demonstrates efficacy of an exemplary peptide of the invention, PN159 to enhance epithelial permeation for a cyclic pentapeptide, melanocortin-4 receptor agonist (MC-4RA) a model oligopeptide agonist for a mammalian cellular receptor. In this example, a combination of one or more of the permeabilizing peptides with MC-4RA is described. Useful formulations in this context can include a combination of an oligopeptide therapeutic, a permeabilizing peptide, and one or more other permeation enhancers. The formulation may also contain buffers, tonicifying agents, pH adjustment agents, and peptide/protein stabilizers such as amino acids, sugars or polyols, polymers, and salts.

The effect of PN159 on permeation of MC-4RA was evaluated in this study. MC-4RA was a methanesulphonate salt with a molecular weight of approximately 1,100 Da, which modulates activity of the MC-4 receptor. The PN159 concentrations evaluated are 5, 25, 50, and 100 μM. 45 mg/ml M-β-CD was used as a solubilizer for all formulations to achieve 10 mg/ml peptide concentration. The effect of PN159 was assessed either by itself or in combination with EDTA (1, 2.5, 5, or 10 mg/ml). The formulation pH was fixed at 4 and the osmolarity was at 220 mOsm/kg.

### HPLC Method

The concentrations of MC-4RA in the basolateral media was analyzed by the RP-HPLC using a C18 RP chromatography with a flow rate of 1 mL/minute and a column temperature of 25° C.
Solvent A: 0.1% TFA in water; Solvent B: 0.1% TFA in ACN
Injection Volume: 50 μL
Detection: 220 nm
RUN TIME: 15 MIN

MC-4RA was combined with 5, 25, 50, and 100 μM PN159, pH 4 and osmolarity ˜220 mOsm/kg. The combination was tested using an in vitro epithelial tissue model to monitor PTH permeation, transepithelial electrical resistance (TER), and the cytotoxicity of the formulation by MTT and LDH assays.

The results of studies of the permeation of MC-4RA evinced that TJMP, in addition to enhancing mucosal permeation for peptide hormone therapeutics, significantly enhanced epithelial permeation for an oligopeptide therapeutic agent.

### Example 21

### Permeation Enhancement by TJMP for a Small Molecule Drug

The present example demonstrates efficacy of an exemplary peptide of the invention, PN159, to enhance epithelial permeation for a small molecule drug, exemplified by the acetylcholinesterase (ACE) inhibitor galantamine. In this example, a combination of one or more of the permeabilizing peptides with a small molecule drug is described. Useful formulations in this context can include a combination of a small molecule drug, a permeabilizing peptide, and one or more other permeation enhancers. The formulation may also contain buffers, tonicifying agents, pH adjustment agents, stabilizers and/or preservatives.

The present invention combines galantamine with PN159 to enhance permeation of galantamine across the nasal mucosa. This increase in drug permeation is unexpected because galantamine is a small molecule that can permeate the nasal epithelial membrane independently. The significant enhancement of galantamine permeation across epithelia mediated by addition of excipients which enhance the permeation of peptides is therefore surprising, on the basis that such excipients would not ordinarily be expected to significantly increase permeation of galantamine across the epithelial tissue layer. The invention therefore will facilitate nasal delivery of galantamine and other small molecule drugs by increasing their bioavailability.

In the present studies, 40 mg/ml galantamine in the lactate salt form was combined with 25, 50, and 100 μM PN159 in solution, pH 5.0 and osmolarity-270 mOsm. The combination was tested using an in vitro epithelial model to monitor galantamine permeation, transepithelial electrical resistance (TER), and the cytotoxicity of the formulation by LDH and MTT assays as described above. Permeation measurements for galantamine were conducted by standard HPLC analysis, as follows.

### HPLC Analysis

Galantamine concentration in the formulation and in the basolateral media (permeation samples) was determined using an isocratic LC (Waters Alliance) method with UV detection.
Column: Waters Symmetry Shield, C18, 5 um, 25×0.46 cm
Mobile phase: 5% ACN in 50 mM ammonium formate, pH 3.0
Flow rate: 1 ml/min
Column temperature: 30° C.
Calibration curve: 0-400 μg/ml Galantamine HBr
Detection: UV at 285 nm

Based on the foregoing studies, PN159 improves transmucosal delivery of small molecules. Galantamine was chosen as a model low molecular weight drug, and the results for this molecule are considered predictive of permeabilizing peptide activity for other small molecule drugs. To evaluate permeabilizing activity in this context, 40 mg/ml galantamine in the lactate salt form was combined with 25, 50, and 100 μM PN159 in solution, pH 5.0 and osmolarity ˜270 mOsm. The combination was tested using an in vitro epithelial tissue model to monitor galantamine permeation, transepithelial electrical resistance (TER), and the cytotoxicity of the formulation by LDH and MTT assays.

In the in vitro tissue model, the addition of PN 159 resulted in a dramatic increase in drug permeation across the cell barrier. Specifically, there was a 2.5-3.5 fold increase in the Pₐₚₚ of 40 mg/ml galantamine.

PN159 reduced TER in the presence of galantamine just as described in previous examples.

Cell viability remained high (>80%) in the presence of galantamine lactate and PN159 at all concentrations tested. Conversely, cytotoxicity was low in the presence of PN159 and galantamine lactate, as measured by LDH. Both of these assays suggest that PN159 is not toxic to the epithelial membrane.

In the absence of PN159, the Pₐₚₚ for galantamine was about 2.1×10⁻⁶ cm/s. In the presence of 25, 50 and 100 mM PN159, Pₐₚₚ was 5.1×10⁻⁶, 6.2×10⁻⁶, and 7.2×10⁻⁶ cm/s, respectively. Thus, the PN159 afforded a 2.4- to 3.4-fold increase in Pₐₚₚ of this model low-molecular-weight drug.

TJMP surprisingly increased epithelial permeation of galantamine as a model low molecular weight drug. The addition of PN159 to galantamine in solution significantly enhanced galantamine permeation across epithelial monolayers. Evidence shows that PN159 temporarily reduced TER across the epithelial membrane without damaging the cells in the membrane, as measured by high cell viability and low cytotoxicity. TJMP enhanced bioavailability of galantamine and other small molecule drugs in vivo via the same mechanism that is demonstrated herein using in vitro models. It is further expected that TJMP will enhance permeation of galantamine at higher concentrations as well.

### Example 22

### Permeation Enhancement by TJMP for Proteins

Having established the utility of the PN159 for transmucosal formulations of low-molecular-weight compounds, it was important to discern whether these observations could be extrapolated to larger molecules, e.g., therapeutic peptides and proteins. For this purpose, in vitro tissue studies were performed on salmon calcitonin as a model therapeutic peptide in the absence and presence of 25, 50, and 100 mM PN159. In the absence of PN159, the Pₐₚₚ for calcitonin was about 1×10⁻⁷ cm/s, about an order of magnitude lower than that for galantamine, presumably due to the difference in molecular weight. The data reveal a dramatic increased in calcitonin permeation in the presence of the PN159, up to a 23- to 47-fold increase in Pₐₚₚ compared to the case of the calcitonin alone (Table 20).

TABLE 20
Pₐₚₚ Measured Using the In Vitro Tissue Model

| | [PN159] | Papp | |
|---|---|---|---|
| Drug Formulation | (μM) | (cm/s) | Relative Pₐₚₚ |
| Galantamine | 0 | 2.1 × 10⁻⁶ | 1.0 |
| 40 mg/mL, pH 5.0 | 25 | 5.1 × 10⁻⁶ | 2.4 |
| | 50 | 6.2 × 10⁻⁶ | 3.0 |
| | 100 | 7.2 × 10⁻⁶ | 3.4 |
| Calcitonin | 0 | 9.7 × 10⁻⁸ | 1.0 |
| 1 mg/mL, pH 3.5 | 25 | 2.2 × 10⁻⁶ | 23. |
| | 50 | 3.3 × 10⁻⁶ | 34. |
| | 100 | 4.6 × 10⁻⁶ | 47. |
| PTH₁₋₃₄ | 0 | 1.1 × 10⁻⁷ | 1.0 |
| 1 mg/mL, pH 4.5 | 25 | 3.4 × 10⁻⁷ | 3.0 |
| | 50 | 4.9 × 10⁻⁷ | 4.5 |
| | 100 | 4.3 × 10⁻⁷ | 3.9 |
| PYY₃₋₃₆ | 0^{a} | 1.3 × 10⁻⁷ | 1.0 |
| 1 mg/mL, pH 7.0 | 25 | 1.6 × 10⁻⁶ | 12. |
| | 100 | 2.2 × 10⁻⁶ | 17. |

^{a}pH was 5.0

In order to explore the generality of these findings, two additional peptides, namely human parathyroid hormone 1-34 (PTH₁₋₃₄) and human peptide YY 3-36 (PYY₃₋₃₆) were examined in the in vitro model in the absence and presence of PN159 (Pₐₚₚ data presented in Table 20). In the absence of PN159, the Pₐₚₚ of these two peptides was consistent to that for calcitonin. In the case of PTH₁₋₃₄, the presence of PN159 afforded about 3-5 fold increase in Pₐₚₚ. When PYY₃₋₃₆ was formulated in the presence of PN159, the Papp was increased about 12- to 17-fold. These data confirm the generality of our finding that the TJMP enhanced transmucosal drug delivery for small molecules and proteins.

### Example 23

### Chemical Stability of TJMP

The chemical stability of the PN159 was determined under therapeutically relevant storage conditions. A stability indicating HPLC method was employed. Solutions (50 mM) were stored at various pH (4.0, 7.3, and 9.0) and temperature (5° C., 25° C., 35° C., 40° C., and 50° C.) conditions. Samples at pH 4 contained 10 mM citrate buffer. Samples at pH 7.3 and 9.0 contained 10 mM phosphate buffer. Storage stability results (including the Arrhenius plot) show that PN159 was most chemically stable at low temperature and pH. For example, at 5° C. and pH 4.0 or pH 7.3, there was essentially 100% recovery of PN159 for six month storage. When the storage temperature was increased to 25° C., there was a 7% and 26% loss of native PN159 for samples at pH 4 or pH 7, respectively, after six months. At pH 9 and/or at elevated temperature, e.g., 40 to 50° C., rapid deterioration of the PN159 ensued. The pH range of 4.0 to 7.3 and the temperature range of refrigerated to ambient are most relevant for intranasal formulations. Therefore, these data support that the TJMP can maintain chemical integrity under storage conditions relevant to IN formulations.

### Example 24

### In Vivo Evaluation of Tight Junction Modulating Peptides in Rabbits by Intranasal Administration

A pharmacokinetic (PK) study in rabbits was performed to evaluate the plasma pharmacokinetic properties of Peptide YY (PYY) with various tight junction modulating peptides (TJMPs) administered via intranasal (IN) delivery.

### Animal Model

In this study, New Zealand White rabbits (Hra: (NZW) SPF) were used as test subjects to evaluate plasma pharmacokinetics of MC-4RA by intranasal administration and intravenous infusion. The treatment of animals was in accordance with regulations outlined in the USDA Animal Welfare Act (9 CFR Parts 1, 2, and 3) and the conditions specified in the Guide for the Care and Use of Laboratory Animals (ILAR publication, 1996, National Academy Press).

Rabbits were chosen as animal subjects for this study because the pharmacokinetic profile derived from a drug administered to rabbits closely resembles the PK profile for the same drug in humans.

### Dose Administration

The experimental design and dosing regime for the 9 TJMPs tested is summarized in Table 21. All experimental groups were given 205 μg/kg PYY(3-36) in combination with an individual TJMP or phosphate buffered saline (PBS; negative control) by intranasal (IN) administration. Each formulation was administered once into the left nares using a pipetteman and disposable plastic tip. The head of the animal was tilted back and the dose was administered at the time of inhalation by the animal so as to allow capillary action to draw the solution into the nares. Following IN administration, the animal's head was restrained in the tilted back position for about 15 seconds to prevent any loss of the administered dose. During the procedure, extreme care was taken to avoid any issue damage potentially resulting from contact with intranasal mucosa.

TABLE 21

| | Number of | | Tight Junction Modulator | PYY3 |
|---|---|---|---|---|
| Group | Animals | Route | (Concentration) | (μg/kg) |
| 1 | 5 M | Intranasal | PBS | 205 |
| 2 | 5 M | Intranasal | PN159 (50 μM) | 205 |
| 3 | 5 M | Intranasal | PN161 (100 μM) | 205 |
| 4 | 5 M | Intranasal | PN202 (100 μM) | 205 |
| 5 | 5 M | Intranasal | PN27 (250 μM) | 205 |
| 6 | 5 M | Intranasal | PN58 (500 μM) | 205 |
| 7 | 5 M | Intranasal | PN73 (500 μM) | 205 |
| 8 | 5 M | Intranasal | PN228 (500 μM) | 205 |
| 9 | 5 M | Intranasal | PN183 (1000 μM) | 205 |
| 10 | 5 M | Intranasal | P7N556 (1000 μM) | 205 |

PN556 has the same primary sequence as PN283, but has no maleimide modification at the N-terminus of the peptide.

### Blood and Plasma Sample Collection

Following does administration by IN, serial blood samples were taken from each animal by direct venipuncture of a marginal ear vein. Blood samples were collected at predose, 5, 10, 15, 20, 30, 45, 60, 90, 120 and 180 minutes post-dosing. Samples were collected in tubes containing dipotassium EDTA as the anticoagulant. The tubes were chilled until centrifugation. All samples were centrifuged within 1 hour of collection. Plasma was harvested and transferred into prelabled plastic vials, frozen in a dry ice/acetone bath, and then stored at approximately −70° C. until a pharmacokinetic analysis was performed.

Clinical observations were made at each blood sampling time and an examination of both nostrils for all animals in the IN administration test groups was conducted just prior to 5 minutes and 1 hour post-intranasal dosing.

### Analytical Method

Samples from each animal in all study groups were analyzed for PYY (3-36) levels using by ELISA. The test articles prior to and after dosing were run on HPLC for quality control. Aliquots (0.1 mL) of plasma were protein precipitated with acetonitrile after adding a bio-analytical internal standard. The supernatant was dried with nitrogen, reconstituted in HPLC buffer and then injected onto a HPLC system. The effluent is detected by positive ion electrospray ionization tandem triple quadrupole mass spectrometer. The PK data was analyzed by WinNonlin (Pharsight Corp., Mountain View).

### Results

The mean plasma PK parameters for each test group are summarized in Table 22. No adverse clinical signs were observed following administration of any formulations. Post-intranasal examination of both nostrils of animals administered formulations via IN revealed neither any redness, nor swelling. The PK study evaluated the Cₘₐₓ (maximum observed concentration), Tₘₐₓ (time of maximum concentration) and AUC (Area Under the Curve) last and infinity (inf). Eight TJMPs were ranked and categorized into 4 different performance tiers according to their level of in vivo permeability with Tier I containing TJMPs with the greatest level of in vivo permeability and each subsequent Tier containing TJMPs with progressively decreasing levels of in vivo permeability.

TABLE 22

| | In Vivo | | | | AUClast | AUCinf |
|---|---|---|---|---|---|---|
| | Tier | | Tₘₐₓ | Cₘₐₓ | (min * pg/ | (min * pg/ |
| Group | Ranking | T_{1/2} | (min) | (pg/mL) | mL) | mL) |
| PBS | | 86.0 | 22.0 | 806 | 4.5 × 10⁴ | 6.81 × 10⁴ |
| PN159 | I | 30.2 | 17.0 | 30200 | 1.52 × 10⁶ | 1.55 × 10⁶ |
| PN161 | I | 34.3 | 24.0 | 32100 | 1.62 × 10⁶ | 1.65 × 10⁶ |
| PN27 | I | 29.9 | 33.0 | 29300 | 1.67 × 10⁶ | 1.71 × 10⁶ |
| PN228 | II | 30.4 | 31.0 | 21200 | 1.06 × 10⁶ | 1.08 × 10⁶ |
| PN202 | II | 34.1 | 32.0 | 12700 | 7.35 × 10⁵ | 7.63 × 10⁵ |
| PN58 | III | 29.5 | 43.0 | 12800 | 8.3 × 10⁵ | 8.71 × 10⁵ |
| PN73 | IV | 53.8 | 37.0 | 8220 | 3.46 × 10⁵ | 3.55 × 10⁵ |
| PN183 | IV | 33.7 | 22.0 | 5450 | 2.58 × 10⁵ | 2.75 × 10⁵ |
| PN556 | IV | 51.2 | 22.0 | 4620 | 2.47 × 10⁵ | 2.80 × 10⁵ |

Theses data shows that the in vivo permeability observed for both PN161 and PN27 is comparable to PN159; and the remaining TJMPs, at the concentrations tested, achieved a level of in vivo permeability below that of PN159.

### Example 25

### Tight Junction Modulating Peptides That Enhance Epithelial Cell Layer Permeation In Vitro

The present example describes the exemplary peptides PN679 and PN745 of the present invention (shown in Table 23) and the test formulation for each peptide (shown in Table 24) screened to determine each peptide's effective concentration range for epithelial cell monolayer permeation enhancement.

TABLE 23
Tight Junction Modulating Peptides

| | | Molecular | | Purity |
|---|---|---|---|---|
| Peptide # | Amino Acid Sequence | Weight | Lot# | (%) |
| PN679 | CNGRCGGKKKLKLLLKLL | 1984.78 | 05-1882-758 | 94.01 |
| | (SEQ ID NO: 32) | | | |
| PN745 | LRKLRKLRLLRLRKLRKRLLR-amide | 2684.53 | 05-1882-761 | 99.29 |
| | (SEQ ID NO: 33) | | | |

Table 24 below describes the individual test formulations containing an exemplary peptide (“Active Agent” column in Table 24) of the present invention and the test formulations that served as either a positive and negative test formulation controls that were examined by TER, LDH (cytotoxicity) and sample permeation enhancement assays. Each peptide was tested at a 25 μM, 100 μM, 250 μM, 500 μM and 1000 μM concentration. PN159 (test formulation #11) herein served as a TJMP positive control and has previously demonstrated the ability to effectively reduce TER and enhance sample permeation at 25 μM. One percent Triton X-100™ (test formulation #14) functioned as a positive control for both the cytotoxicity (LDH) assay and TER reduction assay. “Special sauce” (SS) served herein as a small molecule permeation enhancer. The DPBS++ served as a negative control. Each test formulation had a final volume of 300 μl and a target pH of 7 except test formulation #12, which had a target pH of 5. One percent Triton X-100™ (test formulation #14) functioned as a positive control for the cytotoxicity (LDH) assay.

Of the total 300 μl volume for each test formulation, only a 20 μl sample was applied to the human-derived tracheal/bronchial epithelial cells (EpiAirway™ Tissue model system) in order to assess the effect each test formulation had on TER, LDH and sample permeation.

TABLE 24
Test Formulations

| | | | | 1x | Active | |
|---|---|---|---|---|---|---|
| Test | Active | Treatment | | DPBS++ | Agent | |
| Formulation # | Agent | Concentration | Water | (pH 7.5) | Stock | 10x FD3 |
| 1 | PN679 | 1000 μM | 15 μl | 225 μl | 30 μl | 30 μl |
| 2 | | 500 μM | 30 μl | 225 μl | 15 μl | 30 μl |
| 3 | | 250 μM | 37.5 μl | 225 μl | 7.5 μl | 30 μl |
| 4 | | 100 μM | 42 μl | 225 μl | 3 μl | 30 μl |
| 5 | | 25 μM | 44.3 μl | 225 μl | 0.75 μl | 30 μl |
| 6 | PN745 | 1000 μM | 15 μl | 225 μl | 30 μl | 30 μl |
| 7 | | 500 μM | 30 μl | 225 μl | 15 μl | 30 μl |
| 8 | | 250 μM | 44.9 μl | 225 μl | 0.075 μl | 30 μl |
| 9 | | 100 μM | 44.97 μl | 225 μl | 0.03 μl | 30 μl |
| 10 | | 25 μM | 44.3 μl | 225 μl | 0.75 μl | 30 μl |
| 11 | PN159 | 25 μM | 43.9 μl | 225 μl | 1.1 μl | 30 μl |
| | (Peptide | | | | | |
| | Control) | | | | | |
| 12 | SS | 1X | 120 μl | 0 μl | 150 μl | 30 μl |
| 13 | DPBS++ | 0.75X | 45 μl | 225 μl | 0 μl | 30 μl |
| 14 | Triton X- | 1% | 41.7 μl | 225 μl | 33.33 μl | 0 μl |
| | 100 ™ | | | | | |

SS = “special sauce”

### Example 26

### PN679 and PN745 Modulate Tight Junction Proteins In Vitro

The present example demonstrates that the exemplary peptides PN679 and PN745 effectively reduced TER and significantly enhanced sample permeation in a dose-dependent manner without causing significant cell toxicity indicating that these peptides are effective TJMPs. Table 25 summarizes the TER, LDH and sample permeation (FD3) data for the test formulations described in Table 24 of Example 25. Test formulation #1 for PN679 and test formulation #6 for PN745 were assayed twice. The additional assay results for TER, LDH and sample permeations are shown in parenthesis.

TABLE 25
Summary of TER, LDH and Sample Permeation Enhancement Data

| | | | % Triton-X | |
|---|---|---|---|---|
| Test | | | LDH | % FD3 |
| Formulation # | Active Agent | % T0 TER | Release | Permeation |
| 1 | PN679 | −2% (−2%) | 51% (32%) | 10% (10%) |
| 2 | | −2% | 50% | 10% |
| 3 | | 2% | 38% | 8% |
| 4 | | 7% | 23% | 7% |
| 5 | | 70% | 1% | 0% |
| 6 | PN745 | −3% (−1%) | 45% (32%) | 7% (5%) |
| 7 | | 1% | 45% | 7% |
| 8 | | 1% | 45% | 8% |
| 9 | | 7% | 28% | 6% |
| 10 | | 24% | 11% | 2% |
| 11 | PN159 | 7% | 31% | 8% |
| | (Peptide | | | |
| | Control) | | | |
| 12 | SS | −2% | 27% | 18% |
| 13 | DPBS++ | 91% | 0% | 0% |
| 14 | Triton | | 100% | |
| | X-100 ™ | | | |

SS = “special sauce”

The test formulations including 100 μM, 250 μM, 500 μM and 1000 μM of either of the exemplary peptides PN679 (test formulations #1, #2, #3 and #4) or PN745 (test formulations #6, #7, #8 and #9) of the present invention reduced TER to a degree equivalent to the “special sauce” and significantly below that of the established TJMP control PN159. As expected, the DPBS++negative control did not reduce TER significantly. The ability of both these peptides to reduce TER correlated strongly with their ability to enhance permeation of the FD3 molecule. The 100 μM dose for both PN679 (test formulation #4) and PN745 (test formulation #9) exhibited a percent permeation similar to the PN159 TJMP but with lower cytotoxicity (lower % LDH Release). Higher concentrations of either peptide resulted in increased levels of FD3 permeation above that of PN159, but also increased release of LDH levels indicating increased cytotoxicity. As expected, the DPBS++control did not induce a measurable LDH release. Based on the observed TER reduction, sample permeation and cytotoxicity (LDH release), a 100 μM dose for either the exemplary peptides PN679 and PN745 appear optimal for further analyses for these two TJMPs.

The foregoing data shows the unexpected discovery that the exemplary peptides PN679 and PN745 reduce TER and enhance small molecule permeation without significant toxicity of a human epithelial cell monolayer in vitro. These data indicate that these tight junction modulating peptides (TMJP) are excellent candidates for use in drug delivery across a mucosal surface, for example intranasal (IN) drug delivery.

### Example 27

### Enhanced Permeation In Vitro by a Tight Junction Modulating Peptide Correlates Strongly with Enhanced Permeation Observed In Vivo

A linear regression analysis was performed to determine whether the TJMP permeation kinetics observed in the in vitro EpiAirway epithelial cell model system correlated with the in vivo pharmacokinetic data observed for that same TJMP. To determine if in vitro permeation data functions as a good indicator for success in vivo, the area under the curve-last value (AUC-last) derived from in vivo pharmacokinetic studies done with PYY and TJMPs was plotted against in vitro epithelial cell monolayer permeation studies done with PYY and TJMPs. In vitro permeation was expressed as a percentage and AUC-last as Min*pg/ml. In vitro and in vivo studies for 10 different TJMPs were graphed and a linear regression performed. An R² value of 0.82 (82% correlation) was derived indicating a strong correlation exist for AUC values derived in vivo and percent permeability observed in vitro. Surprisingly, when inter-assay variability is excluded, an R² value of 0.996 (essentially 100%) was derived indicating a direct correlation exist between in vitro permeability and in vivo success. Thus, in vitro permeation can be used to predict in vivo success.

### Example 28

### In Vivo Permeation Enhancement by a TJMP for a Peptide Hormone Therapeutic Agent Equals or Exceeds That of Small Molecule Permeation Enhancers

Twenty male New Zealand White rabbits age 3-6 months and weighing 2.1-3.0 kg were randomly assigned into one of 5 treatment groups with four animals per group. Test animals were dosed at 15 μl/kg and intranasally via pipette. Table 26 below indicates the composition of five different dose groups.

For dosing group 1 (see Table 26) a clinical formulation of PYY including small molecule permeation enhancers was used. The small molecule enhancers in these studies included methyl-β-cyclodextrin, phosphatidylcholine didecanoyl (DDPC), and/or EDTA. Dosing group 2 received PYY dissolved in phosphate buffered saline (PBS). For dosing groups 3-5, various concentrations of PN159 were added to dosing group 2, so that each of dosing groups 3 to 5 consisted of PYY, PN159, and PBS.

TABLE 26
Dosing Groups

| | | | Dose | Dose | PYY |
|---|---|---|---|---|---|
| | | | Conc | Vol | Dose |
| Group | Animals | Permeation enhancers | (mg/ml) | (ml/kg) | (μg/kg) |
| 1 | 4M | Small molecule | 13.67 | 0.015 | 205 |
| | | permeation enhancers | | | |
| 2 | 4M | None | 13.67 | 0.015 | 205 |
| 3 | 4M | 25 μM PN159 | 13.67 | 0.015 | 205 |
| 4 | 4M | 50 μM PN159 | 13.67 | 0.015 | 205 |
| 5 | 4M | 100 μM PN159 | 13.67 | 0.015 | 205 |

Serial blood samples (about 2 ml each) were collected by direct venipuncture from a marginal ear vein into blood collection tubes containing EDTA as an anticoagulant. Blood samples were collected at 0, 2.5, 5, 10, 15, 30, 45, 60, and 120 minutes post-dosing. After collection of the blood, the tubes were gently rocked several times for anti-coagulation, and then 50 μl aprotinin solution was added. The blood was centrifuged at approximately 1,600×g for 15 minutes at approximately 4° C., and plasma samples were dispensed into duplicate aliquots and stored frozen at approximately −70° C.

Averaging all four animals in a treatment group, the following plasma concentrations of PYY were measured (Table 27):

TABLE 27
Summary of PYY Plasma Concentrations for Test Groups

| | Group 1 | | | | |
|---|---|---|---|---|---|
| | Small | Group 2 | | | |
| | molecule | No | Group 3 | Group 4 | Group 5 |
| Time, | permeation | permeation | 25 μM | 50 μM | 100 μM |
| mins | enhancers | enhancers | PN159 | PN159 | PN159 |
| 0 | 183.825 | 257.3 | 228.675 | 424.4 | 294.225 |
| 2.5 | 1280.7 | 242.8 | 526.375 | 749.975 | 1748.225 |
| 5 | 1449.425 | 273.675 | 1430.15 | 1293.4 | 3088.2 |
| 10 | 8251.8 | 372.05 | 6521.7 | 12517.2 | 14486.6 |
| 15 | 13731.2 | 398.225 | 12563.075 | 34455.3 | 20882.725 |
| 30 | 19537.55 | 476.475 | 15222.6 | 35294.375 | 25470.475 |
| 45 | 13036.075 | 340.7 | 9081.125 | 21582.225 | 16499.55 |
| 60 | 7080.875 | 283.825 | 4843.15 | 9461.925 | 10676.625 |
| 120 | 1671.9 | 192.575 | 1224.2 | 2337.775 | 1891.275 |

The pharmacokinetic data calculated from the above data is shown below in Table 28:

TABLE 28
Summary of Pharmacokinetic Data

| Variable | Group | Mean | SD | SE |
|---|---|---|---|---|
| Cmax (pg/mL) | 1 | 19832.18 | 17737.21 | 8868.605 |
| Tmax (min) | 1 | 32.5 | 20.6155 | 10.3078 |
| AUClast | 1 | 991732.1 | 930296.3 | 465148.1 |
| (min * pg/mL) | | | | |
| AUCINF | 1 | 1357132 | 928368.5 | 535993.8 |
| (min * pg/mL) | | | | |
| t½ (min) | 1 | 23.69 | 1.713 | 0.989 |
| Cmax (pg/mL) | 2 | 516.725 | 196.492 | 98.246 |
| Tmax (min) | 2 | 26.25 | 14.3614 | 7.1807 |
| AUClast | 2 | 36475.72 | 9926.104 | 4963.052 |
| (min * pg/mL) | | | | |
| AUCINF | 2 | 60847.41 | 17688.31 | 8844.156 |
| (min * pg/mL) | | | | |
| t½ (min) | 2 | 84.5919 | 26.8859 | 13.4429 |
| Cmax (pg/mL) | 3 | 15533.95 | 13225.88 | 6612.941 |
| Tmax (min) | 3 | 22.5 | 8.6603 | 4.3301 |
| AUClast | 3 | 748104.1 | 661213.8 | 330606.9 |
| (min * pg/mL) | | | | |
| AUCINF | 3 | 796354.7 | 721017.8 | 360508.9 |
| (min * pg/mL) | | | | |
| t½ (min) | 3 | 24.8467 | 4.3108 | 2.1554 |
| Cmax (pg/mL) | 4 | 40995.53 | 32112.71 | 16056.35 |
| Tmax (min) | 4 | 26.25 | 7.5 | 3.75 |
| AUClast | 4 | 1692499 | 1339896 | 669947.8 |
| (min * pg/mL) | | | | |
| AUCINF | 4 | 1787348 | 1395185 | 697592.4 |
| (min * pg/mL) | | | | |
| t½ (min) | 4 | 25.5355 | 8.6139 | 4.3069 |
| Cmax (pg/mL) | 5 | 27974.4 | 17584.31 | 8792.154 |
| Tmax (min) | 5 | 33.75 | 18.8746 | 9.4373 |
| AUClast | 5 | 1384241 | 817758.8 | 408879.4 |
| (min * pg/mL) | | | | |
| AUCINF | 5 | 1518949 | 1030623 | 595030.3 |
| (min * pg/mL) | | | | |
| t½ (min) | 5 | 20.4628 | 6.5069 | 3.7568 |

Compared with the Group 2 (no enhancer) formulation, the following relative enhancement ratios were determined (Table 29):

TABLE 29
Relative Enhancement Ratios

| Group | Formulation | Relative Cmax | Relative AUC last |
|---|---|---|---|
| 1 | Small molecule permeation | 38x | 27x |
| | enhancers | | |
| 3 | PN159, 25 μm | 30x | 21x |
| 4 | PN159, 50 μm | 79x | 46x |
| 5 | PN159, 100 μm | 54x | 38x |

The foregoing data demonstrate that TJMP enhances in vivo intranasal permeation of a human hormone peptide therapeutic to an equal or greater degree compared to small molecule permeation enhancers. The greatest effect of the peptide is seen at a 50 μM concentration. The 100 μM concentration resulted in somewhat less permeation, although both resulted in higher permeation than the small molecule permeation enhancers.

### Example 29

### Permeation Enhancement by TJMP for an Oligo-peptide Therapeutic Agent

The present example demonstrates efficacy of an exemplary peptide of the invention, PN159 to enhance epithelial permeation for a cyclic pentapeptide, melanocortin-4 receptor agonist (MC-4RA) a model oligopeptide agonist for a mammalian cellular receptor. In this example, a combination of one or more of the permeabilizing peptides with MC-4RA is described. Useful formulations in this context can include a combination of an oligopeptide therapeutic, a permeabilizing peptide, and one or more other permeation enhancers. The formulation may also contain buffers, tonicifying agents, pH adjustment agents, and peptide/protein stabilizers such as amino acids, sugars or polyols, polymers, and salts.

The effect of PN159 on permeation of MC-4RA was evaluated in this study. MC-4RA was a methanesulphonate salt with a molecular weight of approximately 1,100 Da, which modulates activity of the MC-4 receptor. The PN159 concentrations evaluated are 5, 25, 50, and 100 μM. 45 mg/ml M-β-CD was used as a solubilizer for all formulations to achieve 10 mg/ml peptide concentration. The effect of PN159 was assessed either by itself or in combination with EDTA (1, 2.5, 5, or 10 mg/ml). The formulation pH was fixed at 4 and the osmolarity was at 220 mOsm/kg.

### HPLC Method

The concentrations of MC-4RA in the basolateral media was analyzed by the RP-HPLC using a C18 RP chromatography with a flow rate of 1 mL/minute and a column temperature of 25° C.
Solvent A: 0.1% TFA in water; Solvent B: 0.1% TFA in ACN
Injection Volume: 50 μL
Detection: 220 nm
RUN TIME: 15 MIN

MC-4RA was combined with 5, 25, 50, and 100 μM PN159, pH 4 and osmolarity ˜220 mOsm/kg. The combination was tested using an in vitro epithelial tissue model to monitor PTH permeation, transepithelial electrical resistance (TER), and the cytotoxicity of the formulation by MTT and LDH assays.

The results of studies of the permeation of MC-4RA evinced that TJMP, in addition to enhancing mucosal permeation for peptide hormone therapeutics, significantly enhanced epithelial permeation for an oligopeptide therapeutic agent.

### Example 30

### Permeation Enhancement by TJMP for a Small Molecule Drug

The present example demonstrates efficacy of an exemplary peptide of the invention, PN159, to enhance epithelial permeation for a small molecule drug, exemplified by the acetylcholinesterase (ACE) inhibitor galantamine. In this example, a combination of one or more of the permeabilizing peptides with a small molecule drug is described. Useful formulations in this context can include a combination of a small molecule drug, a permeabilizing peptide, and one or more other permeation enhancers. The formulation may also contain buffers, tonicifying agents, pH adjustment agents, stabilizers and/or preservatives.

The present invention combines galantamine with PN159 to enhance permeation of galantamine across the nasal mucosa. This increase in drug permeation is unexpected because galantamine is a small molecule that can permeate the nasal epithelial membrane independently. The significant enhancement of galantamine permeation across epithelia mediated by addition of excipients which enhance the permeation of peptides is therefore surprising, on the basis that such excipients would not ordinarily be expected to significantly increase permeation of galantamine across the epithelial tissue layer. The invention therefore will facilitate nasal delivery of galantamine and other small molecule drugs by increasing their bioavailability.

In the present studies, 40 mg/ml galantamine in the lactate salt form was combined with 25, 50, and 100 μM PN159 in solution, pH 5.0 and osmolarity ˜270 mOsm. The combination was tested using an in vitro epithelial tissue model to monitor galantamine permeation, transepithelial electrical resistance (TER), and the cytotoxicity of the formulation by LDH and MTT assays as described above. Permeation measurements for galantamine were conducted by standard HPLC analysis, as follows.

### HPLC Analysis

Galantamine concentration in the formulation and in the basolateral media (permeation samples) was determined using an isocratic LC (Waters Alliance) method with UV detection.
Column: Waters Symmetry Shield, C18, 5 um, 25×0.46 cm
Mobile phase: 5% ACN in 50 mM ammonium formate, pH 3.0
Flow rate: 1 ml/min
Column temperature: 30° C.
Calibration curve: 0-400 μg/ml Galantamine HBr
Detection: UV at 285 nm

Based on the foregoing studies, PN159 improves transmucosal delivery of small molecules. Galantamine was chosen as a model low molecular weight drug, and the results for this molecule are considered predictive of permeabilizing peptide activity for other small molecule drugs. To evaluate permeabilizing activity in this context, 40 mg/ml galantamine in the lactate salt form was combined with 25, 50, and 100 μM PN159 in solution, pH 5.0 and osmolarity ˜270 mOsm. The combination was tested using an in vitro epithelal tissue model to monitor galantamine permeation, transepithelial electrical resistance (TER), and the cytotoxicity of the formulation by LDH and MTT assays.

In the in vitro tissue model, the addition of PN159 resulted in a dramatic increase in drug permeation across the cell barrier. Specifically, there was a 2.5-3.5 fold increase in the Pₐₚₚ of 40 mg/ml galantamine.

PN159 reduced TER in the presence of galantamine just as described in previous examples.

Cell viability remained high (>80%) in the presence of galantamine lactate and PN159 at all concentrations tested. Conversely, cytotoxicity was low in the presence of PN159 and galantamine lactate, as measured by LDH. Both of these assays suggest that PN159 is not toxic to the epithelial membrane.

In the absence of PN159, the Pₐₚₚ for galantamine was about 2.1×10⁻⁶ cm/s. In the presence of 25, 50 and 100 mM PN159, Pₐₚₚ was 5.1×10⁻⁶, 6.2×10⁻⁶, and 7.2×10⁻⁶ cm/s, respectively. Thus, the PN159 afforded a 2.4- to 3.4-fold increase in Pₐₚₚ of this model low-molecular-weight drug.

TJMP surprisingly increased epithelial permeation of galantamine as a model low molecular weight drug. The addition of PN159 to galantamine in solution significantly enhanced galantamine permeation across epithelial monolayers. Evidence shows that PN159 temporarily reduced TER across the epithelial membrane without damaging the cells in the membrane, as measured by high cell viability and low cytotoxicity. TJMP enhanced bioavailability of galantamine and other small molecule drugs in vivo via the same mechanism that is demonstrated herein using in vitro models. It is further expected that TJMP will enhance permeation of galantamine at higher concentrations as well.

### Example 31

### Permeation Enhancement by TJMP for Proteins

Having established the utility of the PN159 for transmucosal formulations of low-molecular-weight compounds, it was important to discern whether these observations could be extrapolated to larger molecules, e.g., therapeutic peptides and proteins. For this purpose, in vitro tissue studies were performed on salmon calcitonin as a model therapeutic peptide in the absence and presence of 25, 50, and 100 mM PN159. In the absence of PN159, the Pₐₚₚ for calcitonin was about 1×10⁻⁷ cm/s, about an order of magnitude lower than that for galantamine, presumably due to the difference in molecular weight. The data reveal a dramatic increased in calcitonin permeation in the presence of the PN159, up to a 23- to 47-fold increase in Pₐₚₚ compared to the case of the calcitonin alone (Table 30).

TABLE 30
Pₐₚₚ Measured Using the In Vitro Tissue Model

| | [PN159] | Papp | |
|---|---|---|---|
| Drug Formulation | (μM) | (cm/s) | Relative Pₐₚₚ |
| Galantamine | 0 | 2.1 × 10⁻⁶ | 1.0 |
| 40 mg/mL, pH 5.0 | 25 | 5.1 × 10⁻⁶ | 2.4 |
| | 50 | 6.2 × 10⁻⁶ | 3.0 |
| | 100 | 7.2 × 10⁻⁶ | 3.4 |
| Calcitonin | 0 | 9.7 × 10⁻⁸ | 1.0 |
| 1 mg/mL, pH 3.5 | 25 | 2.2 × 10⁻⁶ | 23. |
| | 50 | 3.3 × 10⁻⁶ | 34. |
| | 100 | 4.6 × 10⁻⁶ | 47. |
| PTH₁₋₃₄ | 0 | 1.1 × 10⁻⁷ | 1.0 |
| 1 mg/mL, pH 4.5 | 25 | 3.4 × 10⁻⁷ | 3.0 |
| | 50 | 4.9 × 10⁻⁷ | 4.5 |
| | 100 | 4.3 × 10⁻⁷ | 3.9 |
| PYY₃₋₃₆ | 0^{a} | 1.3 × 10⁻⁷ | 1.0 |
| 1 mg/mL, pH 7.0 | 25 | 1.6 × 10⁻⁶ | 12. |
| | 100 | 2.2 × 10⁻⁶ | 17. |

^{a}pH was 5.0

In order to explore the generality of these findings, two additional peptides, namely human parathyroid hormone 1-34 (PTH₁₋₃₄) and human peptide YY 3-36 (PYY₃₋₃₆) were examined in the in vitro model in the absence and presence of PN159 (Pₐₚₚ data presented in Table 30). In the absence of PN159, the Pₐₚₚ of these two peptides was consistent to that for calcitonin. In the case of PTH₁₋₃₄, the presence of PN159 afforded about 3-5 fold increase in Pₐₚₚ. When PYY₃₋₃₆ was formulated in the presence of PN159, the Papp was increased about 12- to 17-fold. These data confirm the generality of our finding that the TJMP enhanced transmucosal drug delivery for small molecules and proteins.

### Example 32

### Chemical Stability of TJMP

The chemical stability of the PN159 was determined under therapeutically relevant storage conditions. A stability indicating HPLC method was employed. Solutions (50 mM) were stored at various pH (4.0, 7.3, and 9.0) and temperature (5° C., 25° C., 35° C., 40° C., and 50° C.) conditions. Samples at pH 4 contained 10 mM citrate buffer. Samples at pH 7.3 and 9.0 contained 10 mM phosphate buffer. Storage stability results (including the Arrhenius plot) show that PN159 was most chemically stable at low temperature and pH. For example, at 5° C. and pH 4.0 or pH7.3, there was essentially 100% recovery of PN159 for six month storage. When the storage temperature was increased to 25° C., there was a 7% and 26% loss of native PN159 for samples at pH 4 or pH 7, respectively, after six months. At pH 9 and/or at elevated temperature, e.g., 40 to 50° C., rapid deterioration of the PN159 ensued. The pH range of 4.0 to 7.3 and the temperature range of refrigerated to ambient are most relevant for intranasal formulations. Therefore, these data support that the TJMP can maintain chemical integrity under storage conditions relevant to IN formulations.

### Example 33

### In Vivo Evaluation of Tight Junction Modulating Peptides in Rabbits by Intranasal Administration

A pharmacokinetic (PK) study in rabbits was performed to evaluate the plasma pharmacokinetic properties of Peptide YY (PYY) with various tight junction modulating peptides (TJMPs) administered via intranasal (IN) delivery.

### Animal Model

In this study, New Zealand White rabbits (Hra: (NZW) SPF) were used as test subjects to evaluate plasma pharmacokinetics of MC-4RA by intranasal administration and intravenous infusion. The treatment of animals was in accordance with regulations outlined in the USDA Animal Welfare Act (9 CFR Parts 1, 2, and 3) and the conditions specified in the Guide for the Care and Use of Laboratory Animals (ILAR publication, 1996, National Academy Press).

Rabbits were chosen as animal subjects for this study because the pharmacokinetic profile derived from a drug administered to rabbits closely resembles the PK profile for the same drug in humans.

### Dose Administration

The experimental design and dosing regime for the 9 TJMPs tested is summarized in Table 31. All experimental groups were given 205 μg/kg PYY(3-36) in combination with an individual TJMP or phosphate buffered saline (PBS; negative control) by intranasal (IN) administration. Each formulation was administered once into the left nares using a pipetteman and disposable plastic tip. The head of the animal was tilted back and the dose was administered at the time of inhalation by the animal so as to allow capillary action to draw the solution into the nares. Following IN administration, the animal's head was restrained in the tilted back position for about 15 seconds to prevent any loss of the administered dose. During the procedure, extreme care was taken to avoid any tissue damage potentially resulting from contact with intranasal mucosa.

TABLE 31
Summary of Test Groups

| | Number of | | Tight Junction Modulator | PYY3 |
|---|---|---|---|---|
| Group | Animals | Route | (Concentration) | (μg/kg) |
| 1 | 5 M | Intranasal | PBS | 205 |
| 2 | 5 M | Intranasal | PN159 (50 μM) | 205 |
| 3 | 5 M | Intranasal | PN161 (100 μM) | 205 |
| 4 | 5 M | Intranasal | PN202 (100 μM) | 205 |
| 5 | 5 M | Intranasal | PN27 (250 μM) | 205 |
| 6 | 5 M | Intranasal | PN58 (500 μM) | 205 |
| 7 | 5 M | Intranasal | PN73 (500 μM) | 205 |
| 8 | 5 M | Intranasal | PN228 (500 μM) | 205 |
| 9 | 5 M | Intranasal | PN183 (1000 μM) | 205 |
| 10 | 5 M | Intranasal | PN556 (1000 μM) | 205 |

PN556 has the same primary sequence as PN283, but has no maleimide modification at the N-terminus of the peptide.

### Blood and Plasma Sample Collection

Following does administration by IN, serial blood samples were taken from each animal by direct venipuncture of a marginal ear vein. Blood samples were collected at predose, 5, 10, 15, 20, 30, 45, 60, 90, 120 and 180 minutes post-dosing. Samples were collected in tubes containing dipotassium EDTA as the anticoagulant. The tubes were chilled until centrifugation. All samples were centrifuged within 1 hour of collection. Plasma was harvested and transferred into prelabled plastic vials, frozen in a dry ice/acetone bath, and then stored at approximately −70° C. until a pharmacokinetic analysis was performed.

Clinical observations were made at each blood sampling time and an examination of both nostrils for all animals in the IN administration test groups was conducted just prior to 5 minutes and 1 hour post-intranasal dosing.

### Analytical Method

Samples from each animal in all study groups were analyzed for PYY (3-36) levels using by ELISA. The test articles prior to and after dosing were run on HPLC for quality control. Aliquots (0.1 mL) of plasma were protein precipitated with acetonitrile after adding a bio-analytical internal standard. The supernatant was dried with nitrogen, reconstituted in HPLC buffer and then injected onto a HPLC system. The effluent is detected by positive ion electrospray ionization tandem triple quadrupole mass spectrometer. The PK data was analyzed by WinNonlin (Pharsight Corp., Mountain View).

### Results

The mean plasma PK parameters for each test group are summarized in Table 32. No adverse clinical signs were observed following administration of any formulations. Post-intranasal examination of both nostrils of animals administered formulations via IN revealed neither any redness, nor swelling. The PK study evaluated the Cₘₐₓ (maximum observed concentration), Tₘₐₓ (time of maximum concentration) and AUC (Area Under the Curve) last and infinity (inf). Eight TJMPs were ranked and categorized into 4 different performance tiers according to their level of in vivo permeability with Tier I containing TJMPs with the greatest level of in vivo permeability and each subsequent Tier containing TJMPs with progressively decreasing levels of in vivo permeability.

TABLE 32
Summary of Pharmacokinetic Data

| | In Vivo | | | | | AUCinf |
|---|---|---|---|---|---|---|
| | Tier | | Tₘₐₓ | Cₘₐₓ | AUClast | (min * pg/ |
| Group | Ranking | T_{1/2} | (min) | (pg/mL) | (min * pg/mL) | mL) |
| PBS | | 86.0 | 22.0 | 806 | 4.5 × 10⁴ | 6.81 × 10⁴ |
| PN159 | I | 30.2 | 17.0 | 30200 | 1.52 × 10⁶ | 1.55 × 10⁶ |
| PN161 | I | 34.3 | 24.0 | 32100 | 1.62 × 10⁶ | 1.65 × 10⁶ |
| PN27 | I | 29.9 | 33.0 | 29300 | 1.67 × 10⁶ | 1.71 × 10⁶ |
| PN228 | II | 30.4 | 31.0 | 21200 | 1.06 × 10⁶ | 1.08 × 10⁶ |
| PN202 | II | 34.1 | 32.0 | 12700 | 7.35 × 10⁵ | 7.63 × 10⁵ |
| PN58 | III | 29.5 | 43.0 | 12800 | 8.3 × 10⁵ | 8.71 × 10⁵ |
| PN73 | IV | 53.8 | 37.0 | 8220 | 3.46 × 10⁵ | 3.55 × 10⁵ |
| PN183 | IV | 33.7 | 22.0 | 5450 | 2.58 × 10⁵ | 2.75 × 10⁵ |
| PN556 | IV | 51.2 | 22.0 | 4620 | 2.47 × 10⁵ | 2.80 × 10⁵ |

### Example 34

### Purification

The following PEGylated PN159 peptides have been synthesized (Table 33):

TABLE 33
List of PEGylated PN0159 Peptides Synthesized.

| | |
|---|---|
| PN526 | (SEQ. ID NO 58) |
| | PEG1-KLALKLALKALKAALKLA-amide |
| PN537 | (SEQ. ID NO 59) |
| | PEG(5000Da)-KLALKLALKALKAALKLA-amide |
| PN570 | (SEQ. ID NO 60) |
| | NH2-KLALKLALKALKAALKLA-PEG1-amide |
| PN571 | (SEQ. ID NO 61) |
| | PEG1-KLALKLALKALKAALKLA-PEG1-amide |
| PN572 | (SEQ. ID NO 62) |
| | PEG3-KLALKLALKALKAALKLA-amide |

A 150 mg quantity of crude peptide was taken up in 15 mL of water containing 0.1% TFA and 3 mL acetic acid. After stirring and sonication, the mixture was transferred to 1.5 mL Eppendorf tubes and centrifuged at 13000 rpm. The supernatant was collected and filtered through a Millex GV 0.22 um syringe filter. This solution was loaded onto a Zorbax 300SB C18 column (21.2 mm ID×250 mm, 7 um particle size) through a 5 mL injection loop at a flow rate of 5 mL/min. The purification was accomplished by running a linear AB gradient of 0.2% B/min where solvent A is 0.1% TFA in water and solvent B is 0.1% TFA in acetonitrile. Under these conditions the peptide eluted over a range of 15-17% B.

### Example 35

### Cells

EpiAirway™ cells (in 96 well format (Air-196-HTS) or individual 24 well insert (Air-100), a human tracheal/bronchial tissue model, was purchased from MatTek Corporation (Ashland, Mass.) to screen for tight junction modulating peptides (TJMPs), based on their effect on transepithelial electrical resistance (TER) and permeability. Cultured tissue was from a single donor and screened negative for HIV, Hepatitis-B, Hepatitis-C, mycoplasma, bacteria, yeast and fungi.

EpiAirway tissues were shipped cold on medium-supplemented agarose gels. The EpiAirway tissues were recovered at 37° C. for 24 hours with medium provided by manufacture. The complete medium (Epi-CM) for EpiAirway models contained DMEM, EFG and other factors, Gentamicin (5 ug/ml), Amphotericin B (0.25 ug/ml) and phenol red as a pH indicator.

### Example 36

### Determination of TER

TER measurement for Air-196-HTS was performed using the Automated Tissue Resistance System (REMS) (World Precession Instrument (WPI), Inc. (Sarasota, Fla.). For monitoring TER in 96 well HTS format, Endhom-Multi(STX) was used in the tissue culture hood to prevent contamination. On overnight recovered inserts, 100 ul medium was used in the apical side and 250 ul in the basal chamber. Background TER was measured with a blank insert (Millipore) and subtracted from tissue inserts. Medium was decanted by inverting the insert onto a paper towel. The insert was then gently tapped on the paper tower to ensure maximum removal of the apical medium. For other TER measurement time points, immediately following treatments, the inserts were gently rinsed with 150 ul Epi-CM three times and drained completely before TER measurement.

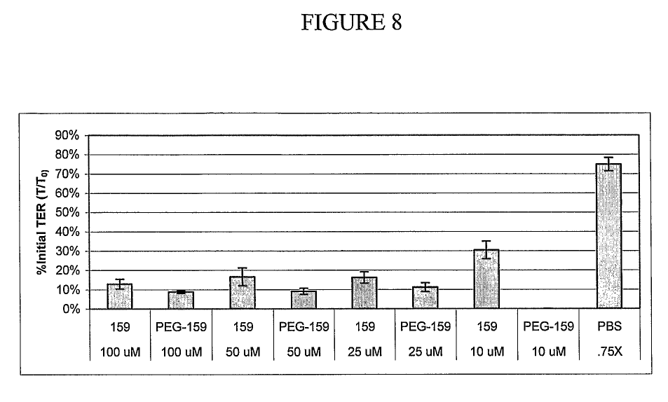

The results (FIG. 8) demonstate that both tight junction modulator peptide PN159 and the PEGyalted version of PN159 of the invention tested on monolayer epithelial cells possess strong, reversible effects for enhancing epithelial permeability. The effects observed with both occur in a predictable manner. Further, the results show that PEG-159 significantly enhances ionic permeability (decreases TER) over PN159 alone. The maximal difference in TER between PEG-PN159 and 159 is at 50 uM PEG-PN159.

### Example 37

### Permeability Assay

Fluorescein isothiocyanate (FITC) labeled Dextrin (MW 3,000) was added to the treatment mixture at 0.1-1 mg/ml. The treatment mixture was added to the side of the apical wall, and the plates were incubated at 37° C. in an orbital shaker (New Brunswick Scientific, Edison, N.J.) for the designated time at 100 rpm. At the end of incubation, triplicates of 200 ul of the basal medium were transferred to a dark-wall fluorescent reading plate. Fluorescent intensity at wavelength 470 nm was measured by a microplate fluorescence reader FLₓ800 (BIO-TEK INSTRUMENTS, INC, Winooski, Vt.). Serial dilutions of standard were used to obtain a standard curve and calculate the concentration. Permeability was measured in two ways, as the ratio of donor mass (the apical chamber) or as the ratio of acceptor mass (the basal chamber), expressed in percentage.

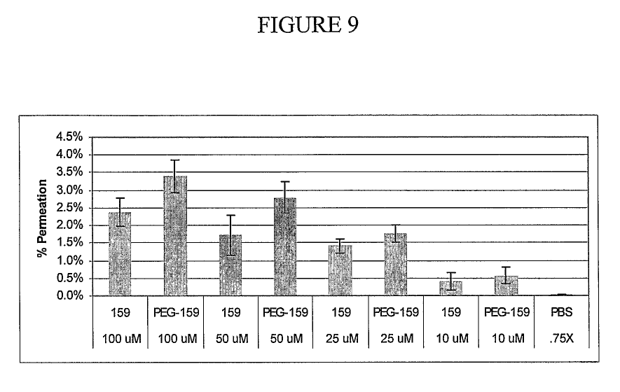

Significant increase in PTH permeation was observed in the presence of both PN159 and the PEG-PN159 of the invention (FIG. 9). The effects observed with both are somewhat concentration dependent between 10 uM and 100 uM. Further, the results show that PEG-PN159 significantly enhances molecular permeability over PN159.

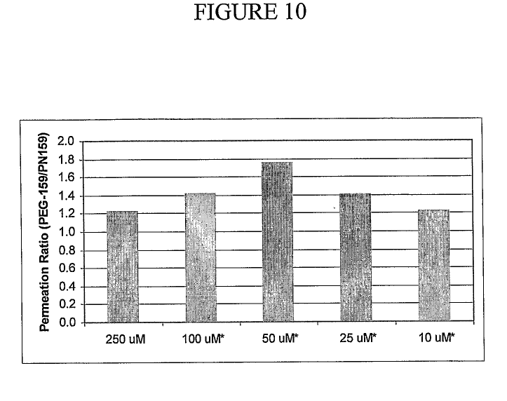

When the permeability increase of PEG-PN159 is compared to PN159 (plotted in FIG. 10 as the ratio between the two values), the maximum differences of permeation increases are at 50 uM concentration.

### Example 38

### Cytotoxicity Assay

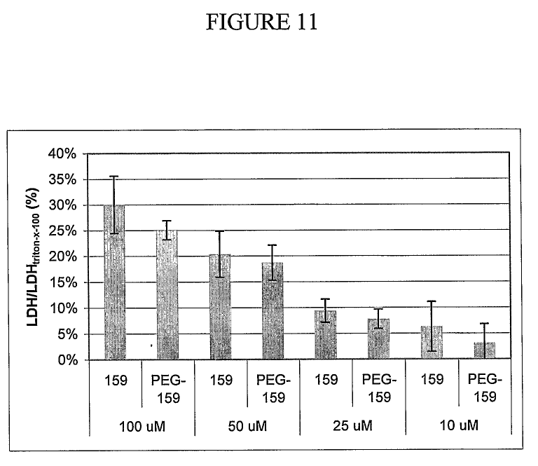

An LDH assay was used to assess the cytotoxicity of the treatments. The LDH level was determined by CytoTox96 Non-Radioactive Cytotoxic Assay (Promega, Madison, Wis.) following the manufacturer's protocol. For basal-lateral LDH levels, triplicates of 50 ul of the basal medium were used to determine the LDH level. For apical LDH level, 150 ul of the diluted apical sample was removed by adding 150 ul of Epi-CM to the apical chamber, the medium was mixed by pipeting up and down, and 150 ul medium was removed and diluted 2× (for a final 8-fold dilution) for assay in triplicates of 50 ul. Total LDH level was determined by lysing cells in a final concentration of 0.9% Triton-X100. The LDH level in each sample was expressed as a percentage of Triton-X100 cell lysis. The results (FIG. 11) show that PEG-PN159 has lower toxicity than PN159.

### Example 39

### Pharmacokinetic Data in Rabbits

Twenty-five male New Zealand White rabbits, approximately 3 months in age, were used in this study. Rabbits received a single intranasal administration, one dose of a tight junction (TJ) peptide and PYY₃₋₃₆ group in one nostril, using a pipetteman and disposable plastic tip. Rabbits were dosed according to the TJ peptide and control groups shown in Table 34. The TJ peptides (PN407, PN408, PN526 (PEG-PN159), and PN159) are all in 0.75×DPBS with calcium and magnesium. The negative control is 0.75×DPBS containing calcium and magnesium only (PBS). A positive PYY3-36 control formulation without TJ peptide contained DDPC, EDTA, and MbCD in citrate buffer was used for comparison (PDF).

The head of the animal was tilted back slightly as the dose was delivered. Following dosing, the head of the animal was restrained in a tilted back position for approximately 15 seconds. Serial blood samples (about 1.5 mL each) were collected by direct venipucture from the marginal ear vein into blood collection tubes containing EDTA as the anticoagulant. Blood samples were collected at 0 (pre-dose), 5, 10, 15, 30, 45, 60, 120 and 240 minutes post dosing for the intranasal groups. After collection the tubes were inverted several times for anti-coagulation. Aprotinin at 50 μL was then added to the collection tubes and mixed gently but thoroughly. Mixed samples were placed on chills packs until centrifugation at approximately 1,600×g for 15 minutes at approximately 4° C. The plasma was split into duplicate aliquots (about 0.35 mL each) and then stored at approximately −70° C.

TABLE 34
Dosing Groups for Rabbit Pharmacokinetic Study

| | Peptide Formulation | PYY₃₋₃₆ | Dose | Dose | |
|---|---|---|---|---|---|
| | and Route of | Dose | Vol | Level | |
| Group | Administration | (mg/mL) | (mL/kg) | (μg/kg) | pH |
| 1 | PN407 Intranasal | 13.67 | 0.015 | 205 | 4.0 |
| 2 | PN408 Intranasal | 13.67 | 0.015 | 205 | 4.0 |
| 3 | PN526 (PEG-PN159) | 13.67 | 0.015 | 205 | 4.0 |
| | Intranasal | | | | |
| 4 | PN159 Intranasal | 13.67 | 0.015 | 205 | 4.0 |
| 5 | Phosphate Buffer | 13.67 | 0.015 | 205 | 4.0 |
| | Solution (PBS) | | | | |
| | Intranasal | | | | |
| 6 | Positive Control (PDF) | 13.67 | 0.015 | 205 | 4.0 |
| | Intranasal | | | | |

The bioanalytical assay of PYY3-36 in rabbit plasma was performed with a commercial ELISA kit (“Active Total Peptide YY (PYY) ELISA”, Cat. No. DSL-10-33600, Diagnostic Systems Laboratories, Inc., Webster, Tex.). The assay is an enzymatically amplified “one-step” sandwich-type immunoassay. In the assay, calibrators, controls, and unknown samples are incubated with anti-PYY antibody in microtitration wells which have been coated with another anti-PYY antibody. After incubation and washing the wells are incubated with the chromogenic substrate, tetramethylbenzidine. An acidic stopping solution is then added and the degree of enzymatic turnover of the substrate is determined by dual wavelength absorbance measurement at 450 and 620 nm. The absorbance measured is proportional to the concentration of PYY present.

A five-parameter logistic data reduction method is applied to the calibrator results to generate a calibration curve for each assay. The calibration curve is used to interpolate PYY concentration values of unknown samples from their absorbance results. Kit components were used for all steps of the assay with the following exceptions: PYY₃₋₃₆ reference material was used to generate the calibrators and controls; calibrators and controls are prepared with stripped (C18 solid phase extraction column) pooled rabbit plasma as diluent; and unknown samples were diluted, if necessary, in stripped pooled rabbit plasma. The antibody combination in this kit was optimized to detect intact human PYY₁₋₃₆, and is fully cross-reactive with mouse PYY₁₋₃₆ and human PYY₃₋₃₆.

Mean pharmacokinetic (PK) data and standard deviations (SD) are presented in Table 35 for controls (PBS and PDF) and TJ Peptides (PN159, PN407, PN408, and PN526) formulations. Relative bioavailability (% BA) for each tight junction modulator and control is presented in Table 36. The percent coefficient of variation for pharmacokinetic variables is presented in Table 37.

TABLE 35
Mean PK Parameters and Standard Deviations (SD) for PYY₃₋₃₆ in Rabbits

| | | Cₘₐₓ | AUCₗₐₛₜ | AUC_{inf} | t½ | Kel |
|---|---|---|---|---|---|---|
| Formulation | Tₘₐₓ (min) | (pg/mL) | (min * pg/mL) | (min * pg/mL) | (min) | (1/min) |
| PBS | 33.75 | 2646.25 | 118438.13 | 147625.18 | 83.12 | 0.009 |
| SD | 18.87 | 1381.06 | 23611.86 | 42331.68 | 22.53 | 0.003 |
| PDF | 30.00 | 19004.40 | 1289219.50 | 1319034.73 | 38.56 | 0.019 |
| SD | 10.61 | 8174.32 | 589127.80 | 612688.59 | 11.12 | 0.005 |
| PN159 | 27.00 | 18346.60 | 973038.80 | 985572.89 | 34.43 | 0.021 |
| SD | 19.56 | 9671.72 | 549668.76 | 546060.77 | 7.20 | 0.005 |
| PN407 | 21.00 | 13980.20 | 725950.50 | 753080.86 | 47.46 | 0.016 |
| SD | 8.22 | 7124.99 | 388368.38 | 397975.49 | 14.51 | 0.004 |
| PN408 | 15.00 | 15420.00 | 721601.50 | 758951.24 | 44.23 | 0.016 |
| SD | 0.00 | 7644.40 | 361013.89 | 360247.20 | 8.23 | 0.003 |
| PN526 | 27.00 | 36066.20 | 1786973.50 | 1819888.30 | 41.04 | 0.018 |
| SD | 6.71 | 22447.13 | 1065867.60 | 1084222.74 | 9.66 | 0.005 |

TABLE 36
% Bioavailability of Tight Junction Modulators

| | AUCₗₐₛₜ | |
|---|---|---|
| Formulation | (min * pg/mL) | % F |
| PBS | 118438.13 | 9.19 |
| PDF | 1289219.50 | |
| PN159 | 973038.80 | 75.48 |
| PN407 | 725950.50 | 56.31 |
| PN408 | 721601.50 | 55.97 |
| PN526 | 1786973.50 | 138.61 |

TABLE 37
% Coefficient of Variation for Pharmacokinetic Parameters

| | | Cₘₐₓ | AUCₗₐₛₜ | AUC_{inf} |
|---|---|---|---|---|
| Formulation | Tₘₐₓ (min) | (pg/mL) | (min * pg/mL) | (min * pg/mL) |
| PBS | 55.9 | 52.2 | 19.9 | 28.7 |
| PDF | 35.4 | 43.0 | 45.7 | 46.4 |
| PN159 | 72.4 | 52.7 | 56.5 | 55.4 |
| PN407 | 39.1 | 51.0 | 53.5 | 52.8 |
| PN408 | 0.0 | 49.6 | 50.0 | 47.5 |
| PN526 | 24.8 | 62.2 | 59.6 | 59.6 |

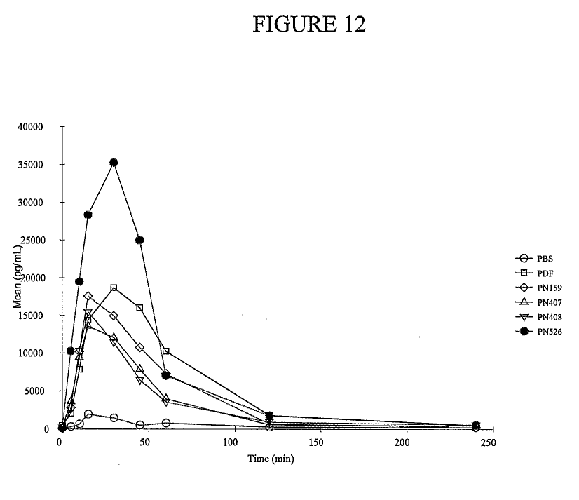

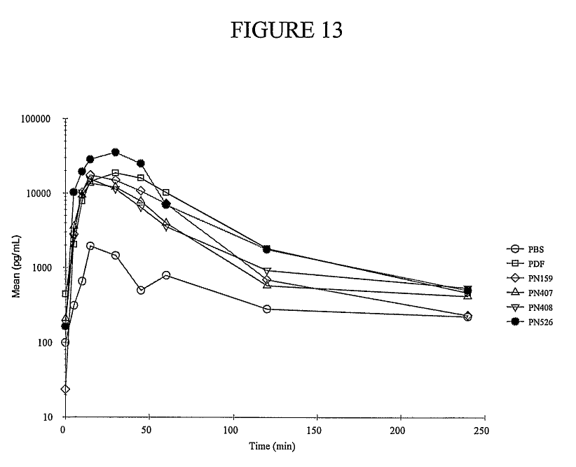

The Lower Limit of Quantification (LLOQ) was considered to be 15.8 pg/mL. Any raw data value that was <NUMBER, was set to 7.9 pg/mL for analysis. Mean PYY₃₋₃₆ plasma concentrations following nasal administration are shown in a Linear Plot in FIG. 12, and a Log-Linear Plot in FIG. 13. Mean serum concentrations of PYY₃₋₃₆ for animals administered the nasal dose indicated peak concentrations (Tₘₐₓ) between 15-34 minutes post-dose for all groups. The mean Cₘₐₓ for the nasal PBS; PDF; PN159; PN407; PN408 and PN526 at a dose level of 205 μg/kg was 2,646.25; 19,004.40; 18,346.60; 13,980.20; 15,420.00 and 36,066.20 pg/mL, respectively. The mean AUCₗₐₛₜ for the nasal PBS; PDF; PN159; PN407; PN408 and PN526 was 118,438.13; 1,289,219.50; 973,038.80; 725,950.50; 721,601.50 and 1,786,973.50 min*pg/mL, respectively. The mean AUC_{inf} for the nasal PBS; PDF; PN159; PN407; PN408 and PN526 was 147,625.18; 1,319,034.73; 985,572.89; 753,080.86; 758,951.24 and 1,819,888.30 min*pg/mL, respectively. The t1/2 was approximately 35-48 minutes for all nasal formulations; however, the PBS was 83 minutes. See Table 35 for a complete list of all pharmacokinetic parameters including standard deviations. The % BA based on AUCₗₐₛₜ for the tight junction modulators versus the PDF formulation were 75, 56, 56 and 139% for PN159, PN407, PN408 and PN526 respectively. The PBS % bioavailability was only 9% compared to the PDF. The coefficient of variation was also compared (Table 37). All tight junction modulators had a similar variation when comparing pharmacokinetic parameters across formulations for Cₘₐₓ, and AUC. The pharmacokinetic variable across all five formulation groups was analyzed using the one-way analysis of variance model and found that the PBS formulation was significantly lower than PN526 for Cₘₐₓ, AUCₗₐₛₜ and AUC_{inf}. (Tₘₐₓ: p=0.27; Cₘₐₓ: p=0.009; AUCₗₐₛₜ: p=0.008; AUC_{inf}: p=0.0097).

Comparing Cₘₐₓ, PEGylated tight junction modulator PN526 was 1.9 fold higher than the PDF and 13.6, 2.6 and 2.3 fold greater than PBS, PN407 and PN408, respectively. Comparing AUCₗₐₛₜ, PEGylated tight junction modulator PN526 was 1.4 fold higher than the PDF and 15.1, 2.5 and 2.5 fold greater than PBS, PN407 and PN408, respectively. The t1/2 was around 40 minutes for all groups, except for the PBS at 80 minutes.

There was a significant difference between the PN526 and the PBS formulation when comparing pharmacokinetic parameters, Cₘₐₓ and AUC; however, there was no significance amongst the tight junction modulators.

Bioavailability was increased with PN526 compared to all other tight junction modulators and the pharmacokinetic parameters were statistically significant compared to the PBS control formulation. These data show that the PEGylated peptide formulation, PN526, has increased % BA above the formulations without PEGylated Peptide, PN159, PN407, PN408, and PBS. Further the % BA for PN526 was also greater than the positive control without PEGylated peptide, PDF.

The examples given herein are solely for the purpose of illustration and are not intended to limit the scope of the invention as described in the claims. Although specific terms and values have been employed herein, such terms and values will be understood as exemplary and non to limit the scope of the invention.

All publications and references cited in this disclosure are hereby incorporated by reference in their entirety for all purposes.

1. A peptide-containing compound or a pharmaceutically-acceptable salt thereof having activity in a mucosa of a mammal to enhance mucosal epithelial transport of an active agent by modulating the permeability of the mucosa, wherein the peptide has a molecular mass of less than 10 kiloDaltons and contains the sequence of PN159 lengthened by one or more amino acids.

2. The compound of claim 1, wherein the peptide is selected from the group consisting of SEQ. ID NOS: 41-43.

3. A peptide-containing compound or a pharmaceutically-acceptable salt thereof having activity in a mucosa of a mammal to enhance mucosal epithelial transport of an active agent by modulating the permeability of the mucosa, wherein the peptide has a molecular mass of less than 10 kiloDaltons and contains the sequence of PN159 having all D-amino acid residues.

4. The compound of claim 3, wherein the peptide is selected from the group consisting of SEQ. ID NO: 35.

5. A peptide-containing compound or a pharmaceutically-acceptable salt thereof having activity in a mucosa of a mammal to enhance mucosal epithelial transport of an active agent by modulating the permeability of the mucosa, wherein the peptide has a molecular mass of less than 10 kiloDaltons and has the retro-inverso sequence of PN159.

6. The compound of claim 5, wherein the peptide is selected from the group consisting of SEQ. ID NO: 38.

7. A peptide-containing compound or a pharmaceutically-acceptable salt thereof having activity in a mucosa of a mammal to enhance mucosal epithelial transport of an active agent by modulating the permeability of the mucosa, wherein the peptide has a molecular mass of less than 10 kiloDaltons and has the sequence of PN159 enriched with at least 60% lysine, leucine, and/or alanine.

8. The compound of claim 7, wherein the peptide is selected from the group consisting of SEQ. ID NOS: 32, 33, 36 and 50.

9. The compound of claims 1, wherein the permeability is enhanced while retaining cell viability in the mucosa.

10. The compound of claim 1, wherein the compound is covalently linked to a water-soluble chain.

11. The compound of claim 10, wherein the chain is a poly(alkylene oxide) chain.

12. The compound of claim 11, wherein the poly(alkylene oxide) chain is branched or unbranched.

13. The compound of claim 12, wherein the poly(alkylene oxide) chain is a polyethylene glycol (PEG) chain.

14. The compound of claim 13, wherein the PEG has a molecular size between about 0.2 and about 200 kiloDaltons (kDa).

15. The compound of claim 13, wherein the PEG has a size less than 40 kDa.

16. The compound of claim 13, wherein the PEG has a size less than 5 kDa.

17. The compound of claim 13, where the poly(alkylene oxide) has a polydispersity value (Mw/Mn) of less than 2.00.

18. The compound of claim 13, wherein the poly(alkylene oxide) has a polydispersity value (Mw/Mn) of less than 1.20.

19. A pharmaceutical formulation comprising a mucosal epithelial transport-enhancing effective amount of a compound of claim 1 and a therapeutically-effective amount of an active agent.

20. The formulation of claim 19, wherein the formulation decreases electrical resistance across a mucosal tissue barrier.

21. The formulation of claim 20, where the decrease in electrical resistance is at least 80%.

22. The formulation of claim 21, wherein the formulation increases permeability of the active agent across a mucosal tissue barrier relative to a similar formulation which does not contain the compound of claim 1.

23. The formulation of claim 22, wherein the increase in permeability is at least two fold.

24. The formulation of claim 22, wherein the permeability is paracellular.

25. The formulation of claim 22, wherein the increased permeability results from modulating a tight junction.

26. The formulation of claim 22, wherein the permeability is transcellular or a mixture of trans- and paracellular.

27. The formulation of claim 22, wherein the mucosal tissue barrier is an epithelial cell layer.

28. The formulation of claim 22, wherein the epithelial cell is selected from the group consisting of tracheal, bronchial, alveolar, nasal, pulmonary, gastrointestinal, epidermal, and buccal.

29. The formulation of claim 22, wherein the epithelial cell is nasal.

30. The formulation of claim 19, wherein the active agent is a peptide, protein, or nucleic acid.

31. The formulation of claim 30, wherein the peptide or protein is comprised of from 2 to 1000 amino acids.

32. The formulation of claim 30, wherein the peptide or protein is comprised of between 2 and 50 amino acids.

33. The formulation of claim 30, wherein the peptide or protein is cyclic.

34. The formulation of claim 30, wherein the peptide or protein is a dimer or oligomer.

35. The formulation of claim 30, wherein the peptide or protein is selected from the group consisting of GLP-1, PYY3-36, PTH1-34 and Exendin-4.

36. The formulation of claim 30, wherein the protein is selected from the group consisting of beta-interferon, alpha-interferon, insulin, erythropoietin, G-CSF, GM-CSF, growth hormone, and analogs thereof.

37. A dosage form comprising the formulation of claim 19, wherein the dosage form is liquid.

38. The dosage form of claim 37, wherein the liquid is in the form of droplets.

39. The dosage form of claim 37, wherein the liquid is in the form of an aerosol.

40. A dosage form comprising the formulation of claim 19, wherein the dosage form is solid.

41. The dosage form of claim 40, wherein the solid is reconstituted in liquid prior to administration.

42. The dosage form of claim 40, wherein the solid is administered as a powder.

43. The dosage form of claim 40, wherein the solid is in the form of a capsule, tablet or gel.

44. A method of administering a molecule to an animal comprising providing a formulation of claim 19 and contacting the formulation with a mucosal surface of the animal.

45. The method of claim 44, wherein the mucosal surface is intranasal.

46. A method of increasing bioavailability of a intranasally-administered active agent in a mammal comprising providing a formulation of claim 19 and administering the formulation to the mammal.

47. The compound of claim 1, wherein the active agent is a siRNA.

48. The compound of claim 1, wherein the active agent is a dsDNA.

49. The compound of claim 1, wherein the active agent is a hematopoietic, an antiinfective; an antidementia; an antiviral, an antitumoral, an antipyretic, an analgesic, an anti-inflammatory, an antiulcerative, an antiallergenic, an antidepressant, a psychotropic, a cardiotonics, an antiarrythmic, a vasodilator, an antihypertensive, a hypotensive diuretic, an antidiabetic, an anticoagulants, a cholesterol-lowering agent, a therapeutic for osteoporosis, a hormone, an antibiotic, or a vaccine.

50. The compound of claim 1, wherein the active agent is a cytokine, a peptide hormone, a growth factor, a cardiovascular factor, a cell adhesion factor, a central or peripheral nervous system factor, a humoral electrolyte factor, a hemal organic substance, a bone growth factor, a gastrointestinal factor, a kidney factor, a connective tissue factor, a sense organ factor, an immune system factor, a respiratory system factor, or a genital organ factor.

51. The compound of claim 1, wherein the active agent is an androgen, an estrogen, a prostaglandin, a somatotropin, a gonadotropin, an interleukin, a steroid, or a cytokine.

52. The compound of claim 1, wherein the active agent is a vaccine for hepatitis, influenza, respiratory syncytial virus (RSV), parainfluenza virus (PIV), tuberculosis, canary pox, chicken pox, measles, mumps, rubella, pneumonia, or human immunodeficiency virus (HIV).

53. The compound of claim 1, wherein the active agent is a bacterial toxoid for diphtheria, tetanus, pseudomonas, or mycobactrium tuberculosis.

54. The compound of claim 1, wherein the active agent is hirugen, hirulos, or hirudine.

55. The compound of claim 1, wherein the active agent is a monoclonal antibody, a polyclonal antibody, a humanized antibody, an antibody fragment, or an immunoglobin.

56. The compound of claim 1, wherein the active agent is morphine, hydromorphone, oxymorphone, lovorphanol, levallorphan, codeine, nalmefene, nalorphine, nalozone, naltrexone, buprenorphine, butorphanol, or nalbufine.

57. The compound of claim 1, wherein the active agent is cortisone, hydrocortisone, fludrocortisone, prednisone, prednisolone, methylprednisolone, triamcinolone, dexamethoasone, betamethoasone, paramethosone, or fluocinolone.

58. The compound of claim 1, wherein the active agent is colchicine, acetaminophen, aspirin, ibuprofen, ketoprofen, indomethacin, naproxen, meloxicam, or piroxicam.

59. The compound of claim 1, wherein the active agent is acyclovir, ribavarin, trifluorothyridine, Ara-A (Arabinofuranosyladenine), acylguanosine, nordeoxyguanosine, azidothymidine, dideoxyadenosine, or dideoxycytidine.

60. The compound of claim 1, wherein the active agent is spironolactone, testosterone, estradiol, progestin, gonadotrophin, estrogen, or progesterone.

61. The compound of claim 1, wherein the active agent is papaverine, nitroglycerin, vasoactive intestinal peptide, calcitonin related gene peptide, cyproheptadine, doxepin, imipramine, cimetidine, dextromethorphan, clozaril, superoxide dismutase, neuroenkephalinase, amphotericin B, griseofulvin, miconazole, ketoconazole, tioconazol, itraconazole, fluconazole, cephalosporin, tetracycline, aminoglucoside, erythromycin, gentamicin, polymyxin B, 5-fluorouracil, bleomycin, methotrexate, and hydroxyurea, dideoxyinosine, floxuridine, 6-mercaptopurine, doxorubicin, daunorubicin, 1-darubicin, taxol, paclitaxel, tocopherol, quinidine, prazosin, verapamil, nifedipine, or diltiazem.

62. The compound of claim 1, wherein the active agent is tissue plasminogen activator (TPA), epidermal growth factor (EGF), fibroblast growth factor (FGF-acidic or basic), platelet derived growth factor (PDGF), transforming growth factor (TGF-alpha or beta), vasoactive intestinal peptide, tumor necrosis factor (TNF), hypothalmic releasing factor, prolactin, thyroid stimulating hormone (TSH), adrenocorticotropic hormone (ACTH), parathyroid hormone (PTH), follicle stimulating hormone (FSF), luteinizing hormone releasing hormone (LHRH), endorphin, glucagon, calcitonin, oxytocin, carbetocin, aldoetecone, enkaphalin, somatostin, somatotropin, somatomedin, alpha-melanocyte stimulating hormone, lidocaine, sufentainil, terbutaline, droperidol, scopolamine, gonadorelin, ciclopirox, buspirone, calcitonin, cromolyn sodium or midazolam, cyclosporin, lisinopril, captopril, delapril, ranitidine, famotidine, superoxide dismutase, asparaginase, arginase, arginine deaminease, adenosine deaminase ribonuclease, trypsin, chemotrypsin, papain, bombesin, substance P, vasopressin, alpha-globulins, transferrin, fibrinogen, beta-lipoprotein, beta-globulin, prothrombin, ceruloplasmin, alpha2-glycoprotein, alpha2-globulin, fetuin, alpha1-lipoprotein, alpha1-globulin, albumin, or prealbumin.

63. A pharmaceutical product comprising a solution containing a compound of claim 1 and an actuator for a mucosal, intranasal, or pulmonary spray.
